(19) United States
(12) Patent Application Publication
(10) Pub. No.: US 2026/0256749 A1
(43) Pub. Date: Sep. 3, 2026

(54) METHODS AND COMPOSITIONS FOR INITIATING, REGULATING, AND MODULATING WEIGHT LOSS AND THERAPEUTIC APPLICATIONS THEREOF

(71) Applicant: Kojin Therapeutics, Inc., Boston, MA (US)

(72) Inventors: Vasanthi Viswanathan, Arlington, MA (US); Priya Chatterji, Boston, MA (US)

(21) Appl. No.: 18/872,492

(22) PCT Filed: Jun. 8, 2023
(86) PCT No.: PCT/US2023/068151

§ 371 Date: Dec. 6, 2024

Related U.S. Application Data
(60) Provisional application No. 63/350,218, filed on Jun. 8, 2022.

(51) Int. Cl. A61K 31/437; A61K 31/137; A61K 31/18; A61K 31/198; A61K 31/36; A61K 31/381; A61K 31/44; A61K 31/455; A61K 31/485; A61K 31/495; A61K 31/496; A61K 31/517; A61K 31/655; A61K 33/04; A61K 38/26; A61P 3/04

(52) U.S. Cl. CPC A61K 31/437; A61K 31/137; A61K 31/18; A61K 31/198; A61K 31/36; A61K 31/381; A61K 31/44; A61K 31/455; A61K 31/485; A61K 31/495; A61K 31/496; A61K 31/517; A61K 31/655; A61K 33/04; A61K 38/26; A61P 3/04

(57) ABSTRACT

Methods and systems and compounds and compositions for the modulation or regulation of weight and/or inducing weight loss and other diseases and conditions in a subject are provided. Various methods of administration are described for modulation or regulation or induction of weight loss and other diseases and conditions are provided. Additionally, methods of weight loss and methods for the treatment of obesity, for example by the modulation of one or more enzymes in the ferroptosis pathway, are provided.

### CROSS REFERENCE TO RELATED APPLICATION

The present application claims priority to U.S. Provisional Application No. 63/350,218, filed Jun. 8, 2022, which is incorporated herein by reference in its entirety.

### SUMMARY

Disclosed herein are methods and compositions for use in inducing weight loss, fat loss, or both, in a subject, the method comprising administering to the subject a ferroptosis inducing agent in an amount sufficient to induce the weight loss, the fat loss, or both. In some embodiments, are methods for use of inducing weight loss. In some embodiments are methods for use of inducing fat loss. In some embodiments are methods for use of inducing weight loss and fat loss. In some embodiments, the method for use further comprises, before the administering to the subject the ferroptosis inducing agent, administering a priming agent to the subject. In some embodiments are methods and compositions for use, wherein an adipose tissue in the subject, after the administrating of the ferroptosis inducing agent to the subject, diminishes by weight, by volume, or both, relative to a weight, volume, or both of the adipose tissue in the subject before the administering. In some embodiments are methods and compositions for use, wherein the adipose tissue in the subject, after the administering to the subject of the ferroptosis inducing agent, diminishes by volume relative to a volume of the adipose tissue before the administering, as determined by a pinch clamp test, by a dual x-ray absorptiometry (DEXA) scan, a diminished waist measurement, a computerized axial tomography (CAT) test, or any combination thereof. In some embodiments are methods and compositions for use, wherein the adipose tissue in the subject, after the administering to the subject of the ferroptosis inducing agent, diminishes by volume relative to a volume of the adipose tissue before the administering, as determined by a pinch clamp test, by a dual x-ray absorptiometry (DEXA) scan, a diminished waist measurement, a computerized axial tomography (CAT) test, or any combination thereof. In some embodiments are methods and compositions for use, wherein the adipose tissue in the subject, after the administering to the subject of the ferroptosis inducing agent, diminishes by weight relative to a weight of the adipose tissue before the administering. In some embodiments are methods and compositions for use, wherein the diminishes by weight is determined by the subject weighing less in total body weight after the administering of the ferroptosis inducing agent as compared to the total body weight of the subject before the administering of the ferroptosis inducing agent. In some embodiments are methods and compositions for use, wherein the diminishes by weight is determined by comparing DEXA scan results of the subject taken before and after the administering to the subject of a ferroptosis inducing agent. In some embodiments are methods and compositions for use, further comprising that a cell size, a cell volume, or both, of a plurality of cells in an adipose tissue of the subject is diminished after the administering to the subject of a ferroptosis inducing agent, relative to a cell size, a cell volume, or both, of a plurality of cells in the adipose tissue of the subject before the administering. In some embodiments are methods and compositions for use, wherein the subject is human. In some embodiments are methods and compositions for use, wherein the ferroptosis inducing agent is administered orally. In some embodiments are methods and compositions for use, the administration occurs for about: 1 day, 2 days, 3 days, 4 days, 5 days, one week, 10 days, two weeks, 15 days, 20, days, three weeks, 25 days, 30 days, one month, two months, three months, four months, five months, six months, one year, two years, three years, four years, five years, for life, or as needed. In some embodiments are methods and compositions for use, wherein the ferroptosis inducing agent is administered in an amount ranging from about 0.1 ng to about 25,000 mg. In some embodiments are methods and compositions for use, wherein the ferroptosis inducing agent is administered in an amount of about: 1 ng, 10 ng, 100 ng, 1 microgram, 10 micrograms, 100 micrograms, 1 mg, 10 mg, 100 mg, 1 g, 2 g, 3 g, 4 g, 5 g, 6 g, 7 g, 8 g, 9 g, 10 g, 15 g, 20 g, or 25 g. In some embodiments are methods and compositions for use, wherein the ferroptosis inducing agent is administered in an amount ranging from about 1 ng/kg to 1,000 mg/kg, wherein mg is mg of the ferroptosis inducing agent, and kg is kg of body weight of the subject. In some embodiments are methods and compositions for use, wherein the ferroptosis inducing agent is administered orally. In some embodiments are methods and compositions for use, wherein the ferroptosis inducing agent is administered by one of the following routes: oral administration, subcutaneous, intradermal, intramuscular, inhalation, intravenous, intraperitoneal, intracranial, intrathecal, transcutaneous, rectal or directly to a fatty tissue. In some embodiments are methods and compositions for use that treats at least one of: obesity, metabolic syndrome, elevated blood glucose, a diabetes, diabetes type 2, diabetes type 3, insulin resistance, high blood pressure, a cardiovascular disease, a coronary artery disease, a cerebrovascular disease, a stroke, a rheumatic heart disease, an arteriosclerosis, an atherosclerosis, a liver disease, a fatty liver disease, a nonalcoholic fatty liver disease (NAFLD), a nonalcoholic steatohepatitis (NASH), or any combination thereof. In some embodiments are methods and compositions for use, wherein the subject is a human male. In some embodiments are methods and compositions for use, wherein the subject is a human female. In some embodiments are methods and compositions for use, wherein the subject ranges from about 1 year of age to about 18 years of age. In some embodiments are methods and compositions for use, wherein the subject ranges from about 18 years of age to about 120 years of age. In some embodiments are methods and compositions for use, wherein the subject is a subject in need thereof. In some embodiments are methods and compositions for use, wherein the ferroptosis inducing agent is comprised in a pharmaceutical composition that comprises a pharmaceutically acceptable: excipient, carrier, or diluent. In some embodiments are methods and compositions for use, wherein the pharmaceutical composition is in unit dose form. In some embodiments are methods and compositions for use, further comprise administering a further therapeutic to the subject. In some embodiments are methods and compositions for use, wherein the further therapeutic is administered concurrently with the ferroptosis inducing agent. In some embodiments are methods and compositions for use, wherein the further therapeutic is administered consecutively with the ferroptosis inducing agent. In some embodiments are methods and compositions for use, wherein the ferroptosis inducing agent is administered directly to a fatty tissue. In some embodiments are methods and compositions for use, wherein the administering is continuously. In some embodiments are methods and compositions for use, wherein the administering is for about: ½ hour, 1 hour, 2 hours, 3 hours, four hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 2 days, 3 days, 4 days, 5 days, 6 days, or one week. In some embodiments are methods and compositions for use, wherein the fatty tissue is heated to a temperature of about; 23, 24, 25, 26, 27, 28, 29, 30, 31, 32, or 33 degrees Celsius. In some embodiments are methods and compositions for use, wherein the ferroptosis inducing agent is administered in the form of an aqueous solution. In some embodiments are methods and compositions for use, wherein a concentration of the ferroptosis inducing agent in the aqueous solution is from about 0.1 nM to about 500 microM. In some embodiments are methods and compositions for use, wherein the administration results in reducing an adipose cell size, or number of adipose cells of the subject, wherein the reduction is determined by measuring the adipose cell size, or number of adipose cells, before and after the administration of the therapeutic amount of the ferroptosis-modulating agent, optionally employing a microscopic evaluation of a biopsy. In some embodiments are methods and compositions for use, wherein the method further comprises reducing an epididymal white adipose tissue of the subject. In some embodiments are methods and compositions for use, wherein the subject is overweight or obese. In some embodiments are methods and compositions for use, wherein the subject is obese and has a BMI of about 30.0 or higher. In some embodiments are methods and compositions for use, wherein the subject is overweight and has a BMI of about 25.0 to about 30. In some embodiments are methods and compositions for use, wherein the further therapeutic has glucagon-like peptide-1 (GLP-1) receptor agonist activity. In some embodiments are methods and compositions for use, wherein the further therapeutic comprises semaglutide, dulaglutide, liraglutide, exenatide, tirzepatide, or any combination thereof.

Also disclosed herein are methods and compositions for use. In some embodiments, disclosed herein are methods and compositions for use, of inducing, regulating, or modulating weight loss, body composition, or fat loss or fat reduction in a subject. In some embodiments, the method comprises administering to the subject a therapeutically effective amount of a ferroptosis-modulating agent, a glutamate-cysteine ligase (GCL) modulating agent, an agent that binds to GCL, an agent that inhibits GCL, or any combination thereof to the subject, thereby inducing, regulating, or modulating the weight loss, body composition, or fat loss or fat reduction in the subject. In some embodiments are methods and compositions for use, wherein the subject is a human. In some embodiments are methods and compositions for use, wherein the subject is a male. In some embodiments are methods and compositions for use, wherein the ferroptosis-modulating agent, the glutamate-cysteine ligase (GCL) modulating agent, the agent that binds to GCL, the agent that inhibits GCL, or any combination thereof is delivered orally as a solution having a concentration of at least about: 5 mg agent per mL of the solution. In some embodiments are methods and compositions for use, wherein the administration is discontinuous and the administration occurs daily for about: 5 days, 10 days, 15 days, 20, days, 25 days, 30 days, two months, three months, four months, five months, six months, or one year. In some embodiments are methods and compositions for use, wherein the ferroptosis inducing agent is selected from the group consisting of: (1S,3R)-RSL3, ML-162, ML-210, JKE-1674, JKE-1716, erastin, jacaric acid, trigonelline, glutamate, sulfasalazine, auranofin, brusatol, sorafenib, sorafenib-d3, sorafenib tosylate, trigonelline, FIN56, FINO2, CIL56, dihydroisotanshinone I, GPX4-IN-3, a sulfoximine, BSO, and a salt of any of these. In some embodiments are methods and compositions for use, In some embodiments are methods and compositions for use, wherein the administration results in reducing an adipose cell size of the subject, wherein the reduction is determined by measuring the adipose cell size before and after the administration of the ferroptosis-inducing agent, optionally employing a microscopic evaluation of a biopsy. In some embodiments are methods and compositions for use, wherein the administration results in reducing the number of adipose cells of the subject, wherein the reduction is determined by measuring the number of adipose cells before and after the administration of the therapeutic amount of the ferroptosis-modulating agent, optionally employing a microscopic evaluation of a biopsy. In some embodiments are methods and compositions for use, wherein the method comprises reducing an epididymal white adipose tissue of the subject. In some embodiments are methods and compositions for use, wherein the subject is overweight or obese. In some embodiments are methods and compositions for use, wherein the subject is obese and has a BMI of about 30.0 or higher. In some embodiments are methods and compositions for use, wherein the subject is overweight and has a BMI of about 25.0 to about 30. In some embodiments are methods and compositions for use, wherein the subject is administered concurrently or consecutively an additional agent or therapy. In some embodiments are methods and compositions for use, wherein the additional agent or therapy is selected from the group consisting of: a GLP-1 agonist, a gastric inhibitory polypeptide analog, naltrexone-bupropion or a salt thereof, orlistat or a salt thereof, phentermine or a salt thereof, phentermine-topiramate or a salt thereof, selenium, an exercise therapy, a surgery, a diet modification, and any combination thereof. In some embodiments are methods and compositions for use, wherein the additional agent or therapy comprises administering the GLP-1 agonist that comprises dulaglutide, exenatide, semaglutide, liraglutide, lixisenatide, or a salt of any of these. In some embodiments are methods and compositions for use, wherein the additional agent or therapy comprises administering the gastric inhibitory polypeptide that comprises tirzepatide or a derivative thereof. In some embodiments are methods and compositions for use, wherein additional agent or therapy comprises the surgery that comprises a gastric sleeve, a gastric bypass, an adjustable gastric band, a ball that is placed in the stomach, or any combination thereof. In some embodiments are methods and compositions for use, wherein the subject has not been diagnosed with a cancer. In some embodiments are methods and compositions for use, In some embodiments are methods and compositions for use, wherein the subject is a subject in need thereof. In some embodiments are methods and compositions for use, wherein the subject has diabetes type I, diabetes type II, diabetes type Illa, or a metabolic syndrome. In some embodiments are methods and compositions for use, wherein the agent is in the form of a pharmaceutical composition that further comprises a pharmaceutically acceptable: carrier, excipient, or diluent. In some embodiments are methods and compositions for use, wherein the pharmaceutical composition is in unit dose form.

Also described herein are methods and compositions for use wherein, the composition comprises i) a ferroptosis-modulating agent, a glutamate-cysteine ligase (GCL) modulating agent, an agent that binds to GCL, an agent that inhibits GCL; and ii) a GLP-1 agonist or a salt thereof, a gastric inhibitory polypeptide analog, a naltrexone-bupropion or a salt thereof, an orlistat or a salt thereof, a phentermine or a salt thereof, and a phentermine-topiramate or a salt thereof, or a selenium or a salt thereof. In some embodiments are methods and compositions for use, wherein the composition comprises i) a glutamate-cysteine ligase (GCL) modulating agent, and ii) a GLP-1 agonist or a salt thereof, a gastric inhibitory polypeptide analog, a naltrexone-bupropion or a salt thereof, an orlistat or a salt thereof, a phentermine or a salt thereof, and a phentermine-topiramate or a salt thereof, or a selenium or a salt thereof. In some embodiments are methods and compositions for use, which are pharmaceutical compositions. In some embodiments are methods and compositions for use, wherein the composition is in unit dose form. In some embodiments are methods and compositions for use in the treatment of a disease or condition. In some embodiments are methods and compositions for use in the treatment of a kidney disease or condition. In some embodiments are methods and compositions for use in the treatment of a symptom associated with a kidney disease or condition. In some embodiments are methods and compositions for use wherein the disease or condition is weight loss, weight maintenance, an iron metabolism associated disease or condition, or a disease or condition associated with high, low, or abnormal cholesterol.

Also described herein are methods and compositions for use in modulating iron metabolism in a plurality of cells in a subject, the method comprising administering to the subject a therapeutically effective amount or an effective amount of a ferroptosis-inducing agent, a glutamate-cysteine ligase (GCL) modulating agent, an agent that binds to GCL, an agent that inhibits GCL, or any combination thereof, thereby modulating the iron metabolism in the subject and thereby inducing ferroptosis in the subject. In some embodiments are methods and compositions for use wherein immediately after the modulating, a majority of cells of the plurality of cells remain alive.

Also described herein are methods and compositions for use in modulating iron metabolism in a plurality of cells in a subject, the method comprising administering to the subject a therapeutically effective amount or an effective amount of a ferroptosis-inducing agent, thereby modulating the iron metabolism in the subject. In some embodiments are methods and compositions for use, wherein immediately after the modulating, a majority of cells of the plurality of cells remain alive.

Also described herein are methods and compositions for use in treating, inducing, regulating, or modulating iron metabolism or a disease or condition associated with iron metabolism in a subject, the method comprising administering to the subject a therapeutically effective amount or an effective amount of a ferroptosis-modulating agent, a glutamate-cysteine ligase (GCL) modulating agent, an agent that binds to GCL, an agent that inhibits GCL, or any combination thereof, thereby treating, inducing, regulating, or modulating the iron metabolism or the disease or condition associated with the iron metabolism in the subject. In some embodiments are methods and compositions for use, wherein the subject is a human. In some embodiments are methods and compositions for use, wherein subject is a subject in need thereof. In some embodiments are methods and compositions for use, wherein a therapeutically effective amount of the ferroptosis-modulating agent is added.

Also described herein are methods and compositions for use in modulating cholesterol metabolism in a subject, the method comprising administering to the subject a therapeutically effective amount or an effective amount of a ferroptosis-modulating agent, a glutamate-cysteine ligase (GCL) modulating agent, an agent that binds to GCL, an agent that inhibits GCL, or any combination thereof, thereby modulating cholesterol metabolism in the subject. In some embodiments are methods and compositions for use, wherein the subject is a human. In some embodiments are methods and compositions for use, wherein subject is a subject in need thereof. In some embodiments are methods and compositions for use, wherein the ferroptosis inducing agent is administered.

Also described herein are methods and compositions for use in modulating cholesterol metabolism in a subject, the method comprising administering to the subject a therapeutically effective amount or an effective amount of a ferroptosis-inducing agent, thereby modulating the cholesterol metabolism in the subject. In some embodiments are methods and compositions for use, wherein the subject is a human. In some embodiments are methods and compositions for use, wherein the subject is a subject in need thereof. In some embodiments are methods and compositions for use, wherein the ferroptosis-inducing agent is administered.

Also described herein are methods and compositions for use in treating, inducing, regulating, modulating, or diminishing cholesterol metabolism or a disease associated with cholesterol metabolism in a subject, the method comprising administering to the subject a therapeutically effective amount or an effective amount of a ferroptosis-modulating agent, a glutamate-cysteine ligase (GCL) modulating agent, an agent that binds to GCL, an agent that inhibits GCL, or any combination thereof, thereby treating, inducing, regulating, modulating, or diminishing the cholesterol metabolism or the disease or condition associated with the cholesterol metabolism in the subject. In some embodiments are methods and compositions for use, wherein the subject is a human. In some embodiments are methods and compositions for use, wherein subject is a subject in need thereof. In some embodiments are methods and compositions for use, wherein the ferroptosis modulating agent is administered.

Also described herein are methods and compositions for use in treating, inducing, regulating, modulating, diminishing, or increasing a reactive oxygen species or a disease or condition associated with a reactive oxygen species in a subject, the method comprising administering to the subject a therapeutically effective amount or an effective amount of a ferroptosis-modulating agent, a glutamate-cysteine ligase (GCL) modulating agent, an agent that binds to GCL, an agent that inhibits GCL, or any combination thereof, thereby treating, inducing, regulating, modulating, diminishing, or increasing the reactive oxygen species or the disease or condition associated with the reactive oxygen species in the subject. In some embodiments are methods and compositions for use, wherein the subject is a human. In some embodiments are methods and compositions for use, wherein subject is a subject in need thereof. In some embodiments are methods and compositions for use, wherein the ferroptosis modulating agent is administered.

Also described herein are methods and compositions for use in increasing a reactive oxygen species in a subject, the method comprising administering to the subject a therapeutically effective amount or an effective amount of a ferroptosis-modulating agent, a glutamate-cysteine ligase (GCL) modulating agent, an agent that binds to GCL, an agent that inhibits GCL, or any combination thereof, thereby increasing the reactive oxygen species in the subject. In some embodiments are methods and compositions for use, wherein the subject is a human. In some embodiments are methods and compositions for use, wherein subject is a subject in need thereof. In some embodiments are methods and compositions for use, wherein the ferroptosis modulating agent is administered.

Also described herein are methods and compositions for use in promoting at least one of: treating, surviving, modulating, or diminishing an acute stress; a chronic stress response; an immune hypersensitivity; cachexia; kidney disease; neurodegeneration; cardiotoxicity; or cardiotoxicity caused at least in part by chemotherapy; in a subject, the method comprising administering to the subject a therapeutically effective amount or an effective amount of a ferroptosis-modulating agent, a glutamate-cysteine ligase (GCL) modulating agent, an agent that binds to GCL, an agent that inhibits GCL, or any combination thereof, thereby treating, inducing, regulating, modulating, diminishing, or increasing the acute stress; a chronic stress response; an immune hypersensitivity; cachexia; kidney disease; neurodegeneration; cardiotoxicity; or cardiotoxicity cause at least in part by chemotherapy in the subject. In some embodiments are methods and compositions for use, wherein the subject is a human. In some embodiments are methods and compositions for use, wherein subject is a subject in need thereof. In some embodiments are methods and compositions for use, wherein the ferroptosis modulating agent is administered.

Also described herein are methods and compositions for use in promoting at least one of: treating a kidney disease in a subject, the method comprising administering to the subject a therapeutically effective amount or an effective amount of a ferroptosis-modulating agent, a glutamate-cysteine ligase (GCL) modulating agent, an agent that binds to GCL, an agent that inhibits GCL, or any combination thereof, thereby treating the kidney disease in the subject. In some embodiments are methods and compositions for use, wherein the subject is a human. In some embodiments are methods and compositions for use, wherein subject is a subject in need thereof. In some embodiments are methods and compositions for use, wherein the ferroptosis modulating agent is administered.

Also described herein are methods and compositions for use in promoting at least one of: treating a kidney disease in a subject, the method comprising administering to the subject a therapeutically effective amount or an effective amount of a ferroptosis-inducing agent, thereby treating the kidney disease in the subject. In some embodiments are methods and compositions for use, wherein the subject is a human. In some embodiments are methods and compositions for use, wherein subject is a subject in need thereof. In some embodiments are methods and compositions for use, wherein the ferroptosis inducing agent is administered. In some embodiments are methods and compositions for use, wherein the method comprises reducing an epididymal white adipose tissue of the subject. In some embodiments are methods and compositions for use, wherein the subject is overweight or obese. In some embodiments are methods and compositions for use, wherein the subject is obese and has a BMI of 30.0 or higher. In some embodiments are methods and compositions for use, wherein the subject is overweight and has a BMI of 25.0 to <30. In some embodiments are methods and compositions for use, wherein the subject is administered concurrently or consecutively an additional agent or therapy. In some embodiments are methods and compositions for use, wherein the additional agent or therapy is selected from the group consisting of: a GLP-1 receptor agonist, a gastric inhibitory polypeptide analog, naltrexone-bupropion or a salt thereof, orlistat or a salt thereof, phentermine or a salt thereof, phentermine-topiramate or a salt thereof, selenium, an exercise therapy, a surgery, a diet modification, and any combination thereof. In some embodiments are methods and compositions for use, wherein the additional agent or therapy comprises administering the GLP-1 receptor agonist that comprises dulaglutide, exenatide, semaglutide, liraglutide, lixisenatide, or a salt of any of these. In some embodiments are methods and compositions for use, wherein the additional agent or therapy comprises administering the gastric inhibitory polypeptide that comprises tirzepatide or a derivative thereof. In some embodiments are methods and compositions for use, wherein additional agent or therapy comprises the surgery that comprises a gastric sleeve, a gastric bypass, an adjustable gastric band, a ball that is placed in the stomach, or any combination thereof. In some embodiments are methods and compositions for use, wherein the subject has not been diagnosed with a cancer. In some embodiments are methods and compositions for use, wherein the subject is a subject in need thereof. In some embodiments are methods and compositions for use, wherein the subject has diabetes type I, diabetes type II, diabetes type Illa, or a metabolic syndrome. In some embodiments are methods and compositions for use, wherein the agent is in the form of a pharmaceutical composition that further comprises a pharmaceutically acceptable: carrier, excipient, or diluent. In some embodiments are methods and compositions for use, wherein the pharmaceutical composition is in unit dose form. In some embodiments are methods and compositions for use, wherein the agent is a compound or a salt thereof in Table 1. In some embodiments are methods and compositions for use, wherein the agent is in the form of a pharmaceutical composition that further comprises a pharmaceutically acceptable: carrier, excipient, or diluent. In some embodiments are methods and compositions for use, wherein the pharmaceutical composition is in unit dose form.

Also described herein are methods and compositions for use including compositions comprising i) a ferroptosis-modulating agent, a glutamate-cysteine ligase (GCL) modulating agent, an agent that binds to GCL, an agent that inhibits GCL; and ii) a GLP-1 agonist or a salt thereof, a gastric inhibitory polypeptide analog, a naltrexone-bupropion or a salt thereof, an orlistat or a salt thereof, a phentermine or a salt thereof, and a phentermine-topiramate or a salt thereof, or a selenium or a salt thereof. In some embodiments are methods and compositions for use, wherein the additional agent or therapy comprises administering the GLP-1 receptor agonist that comprises dulaglutide, exenatide, semaglutide, liraglutide, lixisenatide, or a salt of any of these. In some embodiments are methods and compositions for use, wherein the additional agent or therapy comprises administering the gastric inhibitory polypeptide that comprises tirzepatide or a derivative thereof. In some embodiments are methods and compositions for use, wherein additional agent or therapy comprises the surgery that comprises a gastric sleeve, a gastric bypass, an adjustable gastric band, a ball that is placed in the stomach, or any combination thereof. In some embodiments are methods and compositions for use, wherein the subject has not been diagnosed with a cancer. In some embodiments are methods and compositions for use, wherein the subject is a subject in need thereof. In some embodiments are methods and compositions for use, wherein the subject has diabetes type I, diabetes type II, diabetes type IIIa, or a metabolic syndrome. In some embodiments are methods and compositions for use, wherein the agent is in the form of a pharmaceutical composition that further comprises a pharmaceutically acceptable: carrier, excipient, or diluent. In some embodiments are methods and compositions for use, wherein the pharmaceutical composition is in unit dose form. In some embodiments are methods and compositions for use, wherein the agent is a compound or a salt thereof in Table 1. In some embodiments are methods and compositions for use, wherein the agent is in the form of a pharmaceutical composition that further comprises a pharmaceutically acceptable: carrier, excipient, or diluent. In some embodiments are methods and compositions for use, wherein the pharmaceutical composition is in unit dose form.

Also described herein are methods and compositions for use including compositions comprising i) a ferroptosis-modulating agent, a glutamate-cysteine ligase (GCL) modulating agent, an agent that binds to GCL, an agent that inhibits GCL; and ii) a GLP-1 agonist or a salt thereof, a gastric inhibitory polypeptide analog, a naltrexone-bupropion or a salt thereof, an orlistat or a salt thereof, a phentermine or a salt thereof, and a phentermine-topiramate or a salt thereof, or a selenium or a salt thereof. In some embodiments are methods and compositions for use, which is a pharmaceutical composition. In some embodiments are methods and compositions for use, that is in unit dose form. In some embodiments are methods and compositions for use in the treatment of a disease or condition. In some embodiments are methods and compositions for use in the treatment of a kidney disease or condition. In some embodiments are methods and compositions for use, in the treatment of a symptom associated with a kidney disease or condition. In some embodiments are methods and compositions for use, wherein the disease or condition is weight loss, weight maintenance, an iron metabolism associated disease or condition, or a disease or condition associated with high, low, or abnormal cholesterol.

Also disclosed herein are methods of inducing, regulating, or modulating weight loss in a subject. In some embodiments, the method can comprise administering a therapeutically effective amount of a ferroptosis-modulating agent, a glutamate-cysteine ligase (GCL) modulating agent, an agent that binds to GCL, an agent that inhibits GCL, an agent that modulates an enzyme in a ferroptosis pathway, or any combination thereof to the subject, for example a mammal, for example a mouse, for example a male, thereby inducing, modulating, or regulating weight loss.

Disclosed herein are methods of inducing, regulating, or modulating weight loss in a subject. In some embodiments, the method can comprise administering a therapeutically effective amount of a ferroptosis-modulating agent, a glutamate-cysteine ligase (GCL) modulating agent, an agent that binds to GCL, an agent that inhibits GCL, an agent that modulates an enzyme in a ferroptosis pathway, or any combination thereof to the subject, for example a mammal, for example a human, for example a male, thereby inducing, modulating, or regulating weight loss.

In some embodiments, the agent can be delivered orally, for example as a pill or a capsule or as a solution, or intravenously. When delivered as a solution, the solution can have an concentration of at least about: 5 mg agent per mL of the solution.

In some embodiments, the administration can be discontinuous. The administration can occur daily for about: 5 days, 10 days, 15 days, 20 days, 25 days, 30 days, two months, three months, four months, five months, six months, or one year. In some embodiments, the subject can be a mammal, for example a mouse or a human, and can be a male.

Also disclosed herein is a method of treating, inducing, regulating, or modulating iron metabolism or an iron metabolism associated disease or condition in a subject, the method comprising administering a therapeutically effective amount or effective amount of a ferroptosis-modulating agent, a glutamate-cysteine ligase (GCL) modulating agent, an agent that binds to GCL, an agent that inhibits GCL, or any combination thereof to the subject, thereby treating, inducing, regulating, or modulating the iron metabolism or the iron metabolism associated disease or condition in the subject.

Further disclosed herein is a method of treating, inducing, regulating, modulating, or diminishing cholesterol metabolism or a cholesterol metabolism associated disease or condition in a subject, the method comprising administering a therapeutically effective amount or an effective amount of a ferroptosis-modulating agent, a glutamate-cysteine ligase (GCL) modulating agent, an agent that binds to GCL, an agent that inhibits GCL, or any combination thereof to the subject, thereby treating, inducing, regulating, modulating, or diminishing the cholesterol metabolism or the cholesterol metabolism associated disease or condition in the subject.

Additionally, disclosed herein is a method of treating, inducing, regulating, modulating, diminishing, or increasing a reactive oxygen species or a disease or condition associated with a reactive oxygen species in a subject, the method comprising administering to the subject a therapeutically effective amount of a ferroptosis-modulating agent, a glutamate-cysteine ligase (GCL) modulating agent, an agent that binds to GCL, an agent that inhibits GCL, or any combination thereof, thereby treating, inducing, regulating, modulating, diminishing, or increasing the reactive oxygen species or the disease or condition associated with the reactive oxygen species in the subject.

Further disclosed herein is a method of promoting surviving; or modulating, or diminishing; or treating a disease or condition comprising: an acute stress; a chronic stress or stress response; an apoptosis resistance; an immune hypersensitivity; a cachexia; a kidney disease; a neurodegeneration, a cardiotoxicity, or a cardiotoxicity caused at least in part by chemotherapy; in a subject, the method comprising administering to the subject a therapeutically effective amount or an effective amount of a ferroptosis-modulating agent, a glutamate-cysteine ligase (GCL) modulating agent, an agent that binds to GCL, an agent that inhibits GCL, or any combination thereof thereby promoting surviving; or modulating, or diminishing; or treating the disease or condition in the subject. In some embodiments, the kidney disease comprises one or more of the following disease states or conditions: a multicystic dsplastic kidney, fabry disease, cystinosis, glomerulonephritis, IgA nephropathy, lupus nephritis, atypical hemolytic uremic syndrome (aHUS), a nephrotic syndrome, kidney damage, polycystic kidney disease (PKD), renal artery stenosis, renal tubular acidosis, a simple kidney cyst, or a solitary or single-functioning kidney. In some embodiments, a composition disclosed herein is for use in the treatment of a kidney disease or condition. In some embodiments, a composition disclosed herein is for use in the treatment of a symptom associated with a kidney disease or condition.

In some embodiments, an agent can be a compound or a salt thereof in Table 1.

In some embodiments, the agent can be selected from the group consisting of: (1S,3R)-RSL3, ML-162, ML-210, JKE-1674, JKE-1716, erastin, jacaric acid, trigonelline, glutamate, sulfasalazine, auranofin, brusatol, sorafenib, sorafenib-d3, sorafenib tosylate, trigonelline, FIN56, FINO2, CIL56, dihydroisotanshinone I, GPX4-IN-3, a sulfoximine, buthionine sulfoximine (BSO), or a salt or pharmaceutically acceptable salt of any of these.

In some embodiments, the administration can result in reducing the adipose cell size, cell number, or both, of the subject. In some embodiments, the administration can result in reducing the adipose cell size of the subject. In some embodiments, the administration can result in reducing the number of adipose cells in a subject the subject.

In some embodiments, the reduction can be determined by measuring the adipose cell size before and after the administration of the therapeutic amount of the ferroptosis-modulating agent. Measurement can made, for example, by microscope on a biopsy from a subject.

In some embodiments, the method can comprise reducing the epididymal white adipose tissue of the subject. In some embodiments, the subject can be overweight or obese. In some embodiments, subject can be obese and has a body mass index (BMI) of 30.0 or higher. In some embodiments, the subject can be overweight and has a BMI of 25.0 to <30.

In some embodiments, the subject can be administered concurrently or consecutively a second weight loss therapy. In some embodiments, the second weight loss therapy can be selected from: a GLP-1 agonist, a gastric inhibitory polypeptide analog, a naltrexone-bupropion or a salt thereof, an orlistat or a salt thereof, a phentermine or a salt thereof, and a phentermine-topiramate or a salt thereof, a selenium, an exercise therapy, a surgery, a diet modification, and any combination thereof.

In some embodiments, the second therapy can comprise administering the GLP-1 agonist that comprises a dulaglutide, an exenatide, a semaglutide, a liraglutide, a lixisenatide, an analog, a derivative, or a salt of any of these.

In some embodiments, the second therapy can comprise administering the gastric inhibitory polypeptide that comprises tirzepatide or a derivative thereof.

In some embodiments, the second therapy can comprise the surgery that comprises a gastric sleeve, a gastric bypass, an adjustable gastric band, a ball that is placed in the stomach, or any combination thereof.

In some embodiments, the subject has not been diagnosed with a cancer.

In some embodiments, the subject can be a subject in need thereof. In some embodiments, the subject in need thereof has, is suspected of having, or is diagnosed with diabetes type I, diabetes type II, diabetes type Illa, or a metabolic syndrome. In some embodiments, the subject is at risk of developing diabetes type I, diabetes type II, diabetes type Illa, or a metabolic syndrome.

In some embodiments, the agent is in the form of a pharmaceutical composition that further comprises a pharmaceutically acceptable: carrier, excipient, or diluent.

In some embodiments, the pharmaceutical composition is in unit dose form.

Also described herein are compositions comprising i) the agent, a ferroptosis-modulating or inducing agent, a glutamate-cysteine ligase (GCL) modulating agent, an agent that binds to GCL, or an agent that inhibits GCL; and ii) a GLP-1 agonist or a salt thereof, a gastric inhibitory polypeptide analog, a naltrexone-bupropion or a salt thereof, an orlistat or a salt thereof, a phentermine or a salt thereof, and a phentermine-topiramate or a salt thereof, or a selenium or a salt thereof.

In some embodiments, the composition can be a pharmaceutical composition. In some embodiments, the pharmaceutical composition is in unit dose form.

In some embodiments, the administration of the pharmaceutical composition can be in an effective or therapeutically effective amount and can be orally or intravenously. In some embodiments, the administration once, twice, three, or four times daily, for a day, a week, a month, six months, a year, or longer, or as needed.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a schematic representation of the ferroptosis pathway.

FIG. 2 shows that specific ferroptosis-modulating perturbation resulting in ferroptosis that affects adipose lipid accumulation. The top panels show wild-type (WT) and knock out (KO) adipose tissue images. The bottom graph shows epididymal/gonadal white adipose tissue (mg) in the WT and the KO.

FIG. 3 shows a schematic illustration of NADPH function in the synthesis of lipids and the defense against oxidative stress, for example, as a cofactor of glutathione reductase.

FIGS. 4A-4D shows weight loss change in male controls and males dosed with a ferroptosis agent (FA). FIG. 4A shows a graph of weight change percentage over time in male control mice and male mice treated with 5 mg/ml ferroptosis-inducing agent. FIG. 4B shows a graph of water intake over time in male controls and males dosed with the ferroptosis agent FIG. 4C shows the epididymal white adipose tissue (eWAT) weight for male controls and males dosed with the ferroptosis agent; the water intake of the mouse for male controls and males dosed with the ferroptosis agent. FIG. 4D shows liver GSH concentration levels for male controls and males dosed with the ferroptosis agent.

FIG. 5 shows images of fat cells in control mice and mice treated with 5 mg/ml of a ferroptosis agent. The mice treated with 5 mg/mL of the ferroptosis agent showed decreased adiposity.

FIGS. 6A-6E shows mice treated with a ferroptosis agent when fed on a high fat diet (HFD) did not gain weight. FIG. 6A shows weight change of untreated and a ferroptosis agent treated diet induced obese (DIO) mice when fed a HFD diet. FIG. 6B shows the weight change of C57Bl/6 mice treated with the ferroptosis agent. FIG. 6C shows the eWAT weight of DIO and C57Bl/6 treated and untreated mice. FIGS. 6D-6E shows the lipid profile of DIO and C57Bl/6 treated and untreated mice for triglycerides (FIG. 6D) and LDL cholesterol (FIG. 6E).

FIG. 7 shows a diagram illustrating ferroptosis-modulating perturbation the effect on adipose lipid accumulation.

FIG. 8 shows a diagram illustrating the targets that can enable precise ferroptosis induction.

FIG. 9 shows an overview of ferroptotic death by the process of cellular disintegration due to uncontrolled lipid peroxidation.

FIG. 10 shows experiments indicating ferroptosis sensitivity is associated with apoptosis resistance in melanoma cancer cells (A375) and (PDX), pancreatic cancer cells (KP4), Breast cancer cells (BT474), lung cancer cells (PC9) and ovarian cancer cells (Kuramochi). Cells were treated with different concentrations of a glutathione peroxidase 4 inhibitor (GPX4i).

FIG. 11 shows an illustration that describes ferroptosis sensitivity can be driven by pro-survival stress.

FIG. 12 shows an example of glutathione modulation as an anchor in many different cellular processes that can lead to disease, such as stress defense, neurodegeneration, selenium update, inflammation, infection, and redox signaling.

FIG. 13 shows an illustration of the application of ferroptosis cell states to the treatment of diseases.

FIG. 14 shows induction of ferroptosis in an in vivo tumor. Histological images of a control, ferroptosis-inducer alone and ferroptosis inducer and ferroptosis prevent agent are shown in in vivo cells. The ferroptosis-inducer alone simulated ferroptosis. The ferroptosis inducer and ferroptosis preventing agent inhibited ferroptosis.

### DETAILED DESCRIPTION OF THE DISCLOSURE

The following description and examples illustrate embodiments of the invention in detail. It is to be understood that this invention is not limited to the particular embodiments described herein and as such can vary. Those of skill in the art will recognize that there are numerous variations and modifications of this invention, which are encompassed within its scope.

### Definitions

Throughout this disclosure, various embodiments can be presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

Unless specifically stated or obvious from context, as used herein, the term “about” is understood as within a range of normal tolerance in the art, for example within 2 standard deviations of the mean. “About” can be understood as within 15%, 14%, 13%, 12%, 11%, 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.” Where particular values are described in the application and claims, unless otherwise stated, the term “about” is implicit and in this context means within an acceptable error range for the particular value.

Unless otherwise stated, structures depicted herein are also meant to include all isomeric (e.g., enantiomeric, diastereomeric, geometric (or conformational) forms of the structure; for example, the L and S designations for each asymmetric center, the R and S configurations for each asymmetric center, (Z) and (E) double bond isomers, and (Z) and (E) conformational isomers. Therefore, single stercochemical isomers as well as enantiomeric, diastereomeric, and geometric (or conformational) mixtures of the present compounds are within the scope of the disclosure.

Unless otherwise stated, compounds with one or more asymmetric centers referred to herein, include enantiopure, diastereomeric, and racemic mixtures thereof.

The term “adjacent” and its grammatical equivalents as used herein refer to right next to the object of reference. For example, the term adjacent in the context of a cell or a tissue can mean without any other cells or tissues in between.

The term “analog” and its grammatical equivalents as used herein refer to a molecule that is not identical, but has analogous structural features. An analog of a drug or agent is a drug or agent that is related to a reference agent (e.g., an agent provided in Table 1), but whose chemical structure can be different. Generally, analogues exhibit similar activities to a reference drug or agent, but the activity can be increased or decreased or otherwise improved. Generally, an analogue form of a compound or drug means that the backbone core of the structure is modified or changed compared to a reference drug.

The term “cancer” and its grammatical equivalents as used herein refer to a hyperproliferation of cells whose unique trait—loss of normal controls—results in unregulated growth, lack of differentiation, local tissue invasion, and metastasis. With respect to the methods provided herein, the cancer can be any cancer, including but not limited to any one of: acute lymphocytic cancer, acute myeloid leukemia, alveolar rhabdomyosarcoma, bladder cancer, bone cancer, brain cancer, breast cancer, cancer of the anus, anal canal, rectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, fibrosarcoma, gastrointestinal cancer, Hodgkin lymphoma, hypopharynx cancer, kidney cancer, larynx cancer, leukemia, liquid tumors, liver cancer, lung cancer, lymphoma, malignant mesothelioma, mastocytoma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, colorectal cancer, renal cancer, skin cancer, small intestine cancer, soft tissue cancer, solid tumors, stomach cancer, testicular cancer, thyroid cancer, ureter cancer, and/or urinary bladder cancer. As used herein, the term “tumor” refers to an abnormal growth of cells or tissues, e.g., of malignant type or benign type.

The term “effective amount” or “therapeutically effective amount” and its grammatical equivalents refers to an amount that is sufficient to achieve or at least partially achieve the desired effect.

The term “expression” and its grammatical equivalents as used herein refers to the biosynthesis of a gene product. For example, in the case of a structural gene, expression involves transcription of the structural gene into mRNA and the translation of mRNA into one or more polypeptides.

The term “ferroptosis” refers to a form of cell death involving generation of reactive oxygen species mediated by iron, and characterized by, in part, lipid peroxidation. The term “ferroptosis-inducing agent” or “ferroptosis activator” or “ferroptosis inducer” or “ferroptosis-inducing compound” refers to an agent which promotes or activates ferroptosis in a cell.

The term “hyperproliferative cells” and its grammatical equivalents as used herein refers to cells characterized by unwanted cell proliferation, or abnormally high rate or sustained cell division, unrelated or uncoordinated with that of surrounding normal tissue.

The term “in vitro” and its grammatical equivalents as used herein refers to events that occur in an artificial environment, e.g., in a test tube or reaction vessel, in cell culture, etc., rather than within a multi-cellular organism.

The term “in vivo” and its grammatical equivalents as used herein refers to events that occur within a multi-cellular organism, such as a non-human animal.

The term “iron-dependent cell death agent” and its grammatical equivalents as used herein refers to an agent which induces, promotes or activates cell death mediated by iron. In some cases within the disclosure, the term “iron-dependent cell death agent” is used interchangeably with ferroptosis-inducing agent.

The term “normal cells” and its grammatical equivalents as used herein refers to cells that undergo controlled cell division, controlled activation, or quiescent cells.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

### Overview

Provided herein are compositions and methods useful for treating a disease or condition, such as overweight or obesity. Also provided herein are compositions for use using any of the methods described herein. Provided herein are also treatment regimes for the therapy of various diseases or conditions such as overweight or obesity. A treatment regime can comprise administering an agent or a ferroptosis-modulating agent, alone or in combination with a second therapy, each individually in an effective or therapeutically effective amount, thereby treating the disease or condition. In cases of where two or more agents or active agents are administered, administration can be consecutively or concurrently and separately or in a single dosage form, which can be a fix dose combination drug.

### Ferroptosis

Cell death is a cellular process involved in development, cellular homeostasis, and prevention of proliferative diseases such as cancer. Programmed cell death can take different forms, such as apoptosis, mitotic catastrophe, necrosis, senescence, and autophagy. While each of these processes ultimately lead to cell death, the pathways and mechanisms appear to be unique, both at the molecular and cellular level.

Ferroptosis is a non-apoptotic, oxidative form of regulated cell death involving lipid hydroperoxides and the accumulation of lipid peroxide at the cellular plasma membrane. Cells undergoing ferroptosis do not display the cellular characteristics or functions associated with apoptosis, the canonical form of cell death. Examples of apoptotic cell features include, e.g., mitochondrial cytochrome c release, caspase activation, and chromatin fragmentation. Ferroptosis is also characterized by increased levels of intracellular reactive oxygen species (ROS) which can be prevented by iron chelation and genetic inhibition of cellular iron uptake. Addition of iron, but not by other divalent transition metal ions, can potentiate ferroptosis signaling in cells.

Cellular components implicated in and regulating ferroptosis include, among others, cysteine-glutamate antiporter (system Xc), glutathione peroxidase 4 (GPX4), p53, cargo receptor NCOA4, glutathione synthetase (GSH), glutamate-cysteine ligase (GCL). The inactivation or inhibition of some of these molecules, for example, system Xc, GPX4, or glutathione synthetase leads to iron-dependent cell death or ferroptosis.

Hyperproliferative cells in a drug-resistant state, such as, e.g., drug resistant cancer cells have been found to exhibit a dysregulation in apoptosis cellular pathways. Surprisingly, drug-resistance to apoptotic agents by hyperproliferative cells can have an enhanced ability to undergo ferroptosis. Apoptosis-resistant cells can be killed via ferroptosis induction due to their “flammable” ferroptosis-sensitive state.

In some embodiments, ferroptosis-modulating perturbation can be used to treat diseases and conditions associated with reactive oxygen species, iron metabolism, lipid metabolism, glutathione biosynthesis, cholesterol metabolism, selenium metabolism or any combination thereof.

In some embodiments, ferroptosis-modulating perturbation can affect adipose lipid accumulation. A ferroptosis modulating perturbation can decrease fat accumulation and triglycerides. In some cases, a ferroptosis modulating perturbation can decrease obesity and hepatic fat. A ferroptosis modulating perturbation can lead to metabolic diseases prevention. In some cases, a selenium and/or a ferroptosis modulating perturbation can increase UCP1 expression. In some cases, a selenium and/or a ferroptosis modulating perturbation can increase thermogenesis and energy expenditure which can increase metabolic disease prevention.

### Methods of Characterizing Ferroptosis-Sensitive Cells

Provided herein are methods of identifying and characterizing a ferroptosis-sensitive cell in a subject. In some embodiments, the characterizing is performed prior to treatment of a subject with a ferroptosis-inducing agent provided herein. Ferroptosis-sensitive cells can be identified by the following properties provided herein: (1) a concentration of selenium greater than a selenium concentration in a corresponding normal cell; (2) a concentration of iron greater than an iron concentration in a corresponding normal cell; (3) a polyunsaturated fatty acid (PUFA) concentration greater than a PUFA concentration in a corresponding normal cell; (4) a peroxidizability index (PI) greater than a PI in a corresponding normal tissue; and/or (5) the expression of one or more markers indicative of a mesenchymal state, among other morphological and histological characteristics. Methods of measuring analyte concentrations of selenium, iron, and PUFAs include, e.g., mass spectrometry, chromatography, immunoassays, immunosorbent assays, absorbance and colorimetric assays, and microwave plasma-atomic emission spectroscopy. Methods of measuring markers of a mesenchymal cell state include, e.g., immunoassays, polymerase chain reaction (PCR) assays, and sequencing assays.

### (1) Selenium (Se) Concentration and Selenoproteins

Selenium (Se) is a micronutrient that facilitates the synthesis of selenoproteins in a cell. Dietary selenium is found in meat, nuts, cereals, mushrooms, and vegetables. The selenium content in the human body ranges from about 13 milligrams (mg) to 20 mg. Selenium is involved in the cellular process of selenoprotein synthesis and ferroptosis. Selenoproteins are rare proteins that comprise a selenocysteine (Sec) residue in the place of a cysteine. Non-limiting examples of selenoproteins include GPX1, GPX2, GPX3, GPX4, GPX6, TXNRD1, TXNRD2 (TXRD2), TXNRD3, DIO1, DIO2, DIO3, SEPHS2, SEPS1, SEPP1, SEP15, SEPN1 (SELENON), SEPX1, SEPW1 (SELENOW), SEPT1, SELH, SEL1, SELK, SELM (SELENOM), SELO, and SELV. Selenoproteins exhibit biochemical activities such as oxidoreduction, selenocysteine synthesis, and/or selenium transport. GPX4 is a phospholipid hydroperoxidase that catalyzes the reduction of hydrogen peroxide and organic peroxides, thereby protecting cells against membrane lipid peroxidation, and oxidative stress. GPX4 is a regulator of the ferroptosis pathway and inhibition of GPX4 induces ferroptotic cell death.

Provided herein are methods of identifying a ferroptosis-sensitive cell in a mammalian tissue by the concentration of selenium. In some embodiments, methods provided herein comprise measuring the concentration of selenium (Se) in a cell, a plurality of cells, or a mammalian tissue. In some embodiments, the Se concentration in a cell or the plurality of cells of the mammalian tissue is greater than the Se concentration in cells of healthy tissue by at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100%. In some embodiments, the Se concentration in the plurality of cells of the mammalian tissue is greater than the Se concentration in cells of healthy tissue by 1%-10%, 10%-20%, 20%-30%, 30%-40%, 40%-50%, 50%-60%, 60%-70%, 70%-80%, 80%-90%, or 90%-100%. In some embodiments, methods provided herein comprise administering to a mammal an effective amount of a ferroptosis-inducing agent, wherein a plurality of cells of a mammalian tissue have a selenium concentration greater than the selenium concentration of cells of a normal or healthy tissue; and ferroptosis is induced in the plurality of cells.

### (2) Iron Concentration

Ferroptosis is an iron-dependent cellular process and ferroptosis-sensitive cells have increased concentrations of intracellular iron compared with normal cells. Cells treated with deferoxamine (DFO), an iron chelator used for treating iron overload and an agent reported to block ferroptosis, can inhibit cell death. Alternatively, iron loading into cells by treatment with ferric ammonium citrate (FAC) is sufficient to mimic particle treatment and induce ferroptosis in amino acid-starved cells. Increased iron uptake in cells can lead to the depletion of glutathione, conceivably due to increased ROS generation which results in ferroptosis induction.

Provided herein are methods of identifying a ferroptosis-sensitive cell in a mammalian tissue by the concentration of iron. In some embodiments, methods provided herein comprise measuring the concentration of iron or iron oxide in a cell, a plurality of cells, or a mammalian tissue. In some embodiments, the ferroptosis-sensitive cells comprises an increased intracellular concentration of iron that is at least about 7 parts per billion (ppb) or more, about 8 ppb or more, about 9 ppb or more, about 10 ppb or more, about 20 ppb or more, about 30 ppb or more, about 40 ppb or more, about 50 ppb or more, about 60 ppb or more, about 70 ppb or more, about 80 ppb or more, about 90 ppb or more, about 100 ppb or more, about 110 ppb or more, about 120 ppb or more, about 130 ppb or more, about 140 ppb or more, about 150 ppb or more, about 160 ppb or more, up to 170 ppb. In some embodiments, the ferroptosis-sensitive cells comprise an increased intracellular concentration of iron that is at least about 2 micromolar (μM) or higher, 2.5 μM or higher, 3.0 μM or higher, 4.0 μM or higher, 5.0 μM or higher, up to 10 μM higher than that of normal cells. In some embodiments, the iron concentration in a cell or the plurality of cells of the mammalian tissue is greater than the iron concentration in cells of healthy tissue by at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100%. In some embodiments, the iron concentration in the plurality of cells of the mammalian tissue is greater than the iron concentration in cells of healthy tissue by 1%-10%, 10%-20%, 20%-30%, 30%-40%, 40%-50%, 50%-60%, 60%-70%, 70%-80%, 80%-90%, or 90%-100%. In some embodiments, methods provided herein comprise administering to a mammal an effective amount of a ferroptosis-inducing agent, wherein a plurality of cells of a mammalian tissue has an iron concentration greater than the iron concentration of cells of a normal or healthy tissue; and ferroptosis is induced in the plurality of cells. In some embodiments, methods provided herein comprise administering to a mammal an effective amount of a ferroptosis-inducing agent, wherein a plurality of cells of a mammalian tissue has an iron concentration greater than the iron concentration of cells of a normal or healthy tissue, thereby affecting iron metabolism; and ferroptosis is induced in the plurality of cells.

### (3) PUFA Status

Apoptosis-resistant cells gain advantages by being in a ferroptosis-sensitive state with high levels of polyunsaturated fatty acids (PUFA). Apoptosis-resistant cells can be killed via ferroptosis induction due to their “flammable” high-PUFA state. The flammable state is defined by high membrane abundance of PUFAs (vs. MUFA, monosaturated fatty acids), which are prone to uncontrolled lipid peroxidation—a radical chain reaction of polyunsaturated fatty acids—that leads to ferroptotic cell death.

PUFAs are categorized as omega-3 (n-3) and omega-6 (n-6) depending on the location of the last double bond with reference to the terminal methyl end of the molecule. Non-limiting examples of PUF As include: hexadecatrienoic acid (HTA), alpha-linolenic acid (ALA), stearidonic acid (SDA), eicosatrienoic acid (ETE), eicosatetraenoic acid (ETA), eicosapentaenoic acid (EPA, Timnodonic acid), heneicosapentaenoic acid (HPA), docosapentaenoic acid (DPA, Clupanodonic acid), docosahexaenoic acid (DHA, Cervonic acid), tetracosahexaenoic acid (Nisinic acid), tetracosapentaenoic acid, linoleic acid (LA), gamma-linolenic acid (GLA), eicosadienoic acid, dihomo-gamma-linolenic acid (DGLA), arachidonic acid (AA), docosadienoic acid, adrenic acid (AdA), docosapentaenoic acid (Osbond acid), tetracosatetraenoic acid, and tetracosapentaenoic acid. Humans can synthesize all fatty acids utilized by the body except for linoleic acid (LA, C18:20-6) and alpha-linolenic acid (ALA, C18:3n-3).

Provided herein are methods of identifying a ferroptosis-sensitive cell in a mammalian tissue by the concentration of PUFAs. In some embodiments, methods provided herein comprise administering to a mammal an effective amount of a ferroptosis-inducing agent, wherein a plurality of cells of a mammalian tissue has a polyunsaturated fatty acid (PUFA) concentration greater than the PUFA concentration of cells of a normal or healthy tissue; and ferroptosis is induced in the plurality of cells. In some embodiments, the PUFA concentration in the plurality of cells of the mammalian tissue is greater than the PUFA concentration in cells of healthy or non-malignant tissue of the mammal. In some embodiments, the PUFA concentration in the plurality of cells of the mammalian tissue is greater than the PUFA concentration in cells of healthy tissue by at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100%. In some embodiments, the PUFA concentration in the plurality of cells of the mammalian tissue is greater than the PUFA concentration in cells of healthy tissue by 1%-10%, 10%-20%, 20%-30%, 30%-40%, 40%-50%, 50%-60%, 60%-70%, 70%-80%, 80%-90%, or 90%-100%. In some embodiments, the PUFA concentration in the plurality of cells of the mammalian tissue is greater than a predetermined PUFA concentration. In some embodiments, the predetermined PUFA concentration is about 5, 10, 15, 20, 25, 30, 35, 40, 45, 50, 55, 60, 65, 70, 75, 80, 85, or 90 mole percent of total lipids. In some embodiments, the predetermined PUFA concentration is about 10-20, 20-30, 30-40, 40-50, 50-60, 60-70, 70-80, or 80-90 mole percent of total lipids. In some embodiments, the predetermined PUFA concentration is about 20 mole percent of total lipids.

### (4) PI Index

Cell membrane composition must contain a sufficient threshold of polyunsaturated fatty acyl chains to support enzymatic and/or non-enzymatic lipid peroxidation. The peroxidizability of polyunsaturated fatty acids (PUFAs) is linearly dependent on the number of doubly allylic positions present in the molecules. The susceptibility of a cellular membrane to lipid peroxidation can be estimated using the peroxidizability index (PI), which is calculated from measured fatty acid composition (%, w/w) as follows: PI=(% dienoic×1)+(% trienoic×2)+(% tetraenoic×3)+(% pentaenoic×4)+(% hexaenoic×5). Alternatively, PI can be calculated as: PI=(% monoenoic acids×0.025)+(% dienoic acids×1)+(% trienoic acids×2)+(% tetraenoic acids×4)+(pentaenoic acids×6)+(hexaenoic acid×8). Lipidomic measurements of cellular membrane composition are used to determine the peroxidizability index. Cell lines with low PI values (<50) have low sensitivity to ferroptosis-inducing perturbations (e.g., GPX4 inhibition, GSH depletion, addition of pro-oxidant compounds). Cells are more susceptible to undergoing ferroptosis with increasing membrane PI values.

Cells grown in vitro have fatty acid profiles unlike those of cells in vivo and lower PI levels. Vertebrate cells are unable to synthesize PUFAs de novo and rely on dietary sources for such molecules. Typical cell culture methods use media supplemented with serum (typically 10%, v/v), which is the only source of exogenous lipids and contains 1% of the PUFAs available to cells in the body. As a result, cells grown in culture have half the PUFA levels of cells in vivo and double the amount of monounsaturated fatty acids (MUFAs).

The ferroptosis sensitivity of cell lines can be modulated by inclusion of fatty acids in the culture medium. Saturated fatty acids (SFAs), monounsaturated fatty acids (MUFAs), and deuterated PUF As protect cells from undergoing ferroptosis while the addition of PUFAs increases cell sensitivity to ferroptosis-inducing perturbations. Supplementation of cell culture media with exogenous PUFAs can simulate in vivo PUFA concentrations and induce membrane compositions with higher PI values. Modulatory profiling assays with fatty acid supplementation and ferroptosis inducers allows for the experimental determination of specific membrane PUFA content and PI values sufficient for ferroptosis for a given cell line. For example, the peroxidizability index (PI) of sarcoma and other cancer cells is greater than nonmalignant tissue due to preferential uptake of PUFAs. Many sarcomas preferentially uptake PUFAs and incorporate polyunsaturated fatty acyl chains into membrane lipids, resulting in higher membrane peroxidizability index values (PI>100) versus nonmalignant tissue (average PI=91). The difference in membrane peroxidizability provides a therapeutic window for ferroptosis induction to selectively target sarcoma cells versus nonmalignant tissue. The more peroxidizable membrane state is consistent with observations of higher levels of lipid peroxidative stress in primary bone and soft tissue sarcoma. Addition of exogenous PUFAs can increase oxidative stress in osteogenic sarcoma cells and exhibit selective cytotoxic effects.

Provided herein are methods of identifying a ferroptosis-sensitive cell in a mammalian tissue by the peroxidizability index (PI). In some embodiments, methods provided herein comprise administering to a mammal an effective amount of a ferroptosis-inducing agent, wherein a plurality of cells of the mammalian tissue have a PI greater than the PI in cells of normal or healthy tissue; and ferroptosis is induced in the plurality of cells. In some embodiments, the PI in the plurality of cells of the mammalian tissue is greater than a predetermined PI. In some embodiments, the predetermined PI is about 50, 60, 70, 80, 90, 100, 110, 120, 130, 140, or 150. In some embodiments, the predetermined PI is about 90. In some embodiments, the PI in the plurality of cells of the mammalian tissue is greater than the PI in cells of healthy or non-malignant tissue by about 1%-10%, 10%-20%, 20%-30%, 30%-40%, 40%-50%, 50%-60%, 60%-70%, 70%-80%, 80%-90%, or 90%-100%.

### (5) Mesenchymal Cell State

Therapy-resistant cells have three cellular and patient-derived signatures of high mesenchymal state. The first cellular signature is the expression of mesenchymal cell markers. Ferroptosis-sensitive cells exhibit a one or more marker of a mesenchymal cell state. Mesenchymal cell state markers that can be used to identify a ferroptosis-sensitive cell include but are not limited to: ZEB1, ACSL4, FADS2, PPARγ, Fsp1, SLC7A11, SLC3A2, and LPCAT3. The second cellular signature of a ferroptosis-sensitive cell is the reduced expression of endothelial cell markers as compared to normal cells. Non-limiting examples of endothelial cell markers include: vimentin, E-cadherin, and beta (β)-actin. The third cellular signature of a ferroptosis-sensitive cell is the sensitivity to GPX4 knockdown leading to cell death. GPX4 dependency is more pronounced in cancer cells adopting a therapy-resistant mesenchymal state as compared to normal mesenchymal cell lines. Methods of reducing or silencing GPX4 expression can be achieved, e.g., by CRISPR/Cas9, siRNA or shRNA, among others.

Provided herein are methods of identifying a ferroptosis-sensitive cell in a mammalian tissue by the expression of one or more mesenchymal cell state markers. In some embodiments, the methods provided herein comprise administering to a mammal an effective amount of a ferroptosis-inducing agent, wherein a plurality of cells of a mammalian tissue express one or more markers of a mesenchymal cell state; and ferroptosis is induced in the plurality of cells. In some embodiments, the expression of the mesenchymal cell marker in the plurality of cells of the mammalian tissue is greater than the expression of the mesenchymal cell marker in cells of healthy or non-malignant tissue by about 1%-10%, 10%-20%, 20%-30%, 30%-40%, 40%-50%, 50%-60%, 60%-70%, 70%-80%, 80%-90%, or 90%-100%.

### (6) Additional Morphological Characteristics of Ferroptosis

Cells undergoing ferroptosis are characterized morphologically by the presence of smaller than normal mitochondria with condensed mitochondrial membrane densities, reduction or vanishing of mitochondria crista, and outer mitochondrial membrane rupture. Histology and immunoassays can be used to determine whether a tissue is cancerous, exhibits hyperplasia, or fibrosis, as well as identify ferroptosis-sensitive cells within a mammalian tissue. The cell membrane of cells in a ferroptotic state lack of rupture and blebbing of the plasma membrane normally associated with apoptosis. The nuclear size of ferroptotic cells is normal and lacks chromatin condensation.

In some embodiments, the methods provided herein comprise a step of obtaining a biological sample (e.g., blood sample or tissue biopsy) from a subject. In some embodiments, the methods provided herein further comprise fixing, processing, embedding, sectioning, and staining the biological sample for histological analysis. In some embodiments, the tissue comprises a histological abnormality. In some embodiments, the histological abnormality is determined by a tissue biopsy prior to or during the targeted, sustained administration of the ferroptosis-inducing agent to the tissue. In some embodiments, the histological abnormality is hyperplasia, vascularization/angiogenesis, or fibrosis. Hyperplasia is identified by an increased number of cells in a tissue as compared to a normal healthy tissue. Vascularization and angiogenesis are identified in a tissue sample by immunoassays for vascular markers, e.g., vascular endothelial growth factor (VEGF) and angiopoietin-2 (Ang2). Fibrosis is characterized by abnormal collagen deposits between cells identified in a tissue sample, e.g., by Masson's trichrome, Sirius red, or collagen staining.

### Cell-Death and Ferroptosis-Inducing Agents

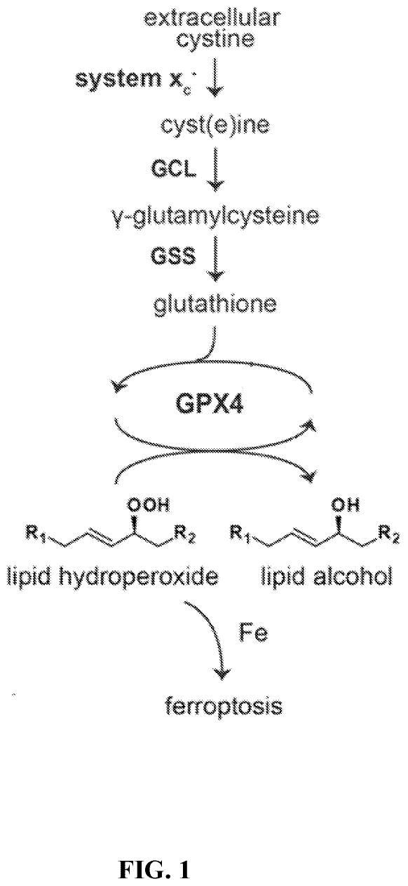

Provided herein are methods of inducing ferroptosis in a tissue in a subject, wherein the methods comprise: (a) sustained administration of a therapeutic amount of a ferroptosis-inducing agent; (b) contacting a tissue in vivo with an effective amount of an iron-dependent cell death agent for a duration of time; and/or (c) contacting a mammalian tissue with a priming agent and then contacting the mammalian tissue in vivo with an effective amount of a ferroptosis-inducing agent for a duration of time. Exemplary targets in the ferroptosis pathway are provided in FIG. 1.

The methods provided herein comprise administering to a cell, tissue, or subject an agent that modulates cell death. In some embodiments, the administering induces cell death. In some embodiments, the administering inhibits or rescues a cell from cell death. In some embodiments, the administering modulates ferroptosis. In some embodiments, the administering induces ferroptosis in vivo. In some embodiments, the administering inhibits ferroptosis in vivo. In some embodiments, the agent is a ferroptosis-inducing agent. In some embodiments, the agent is an iron-dependent cell death inducing agent. Agents useful in the induction of ferroptosis in vivo and for the treatment of a disease or disorder are discussed in further detail below.

### (1) Ferroptosis-Modulating Agents

Provided herein are agents that modulate ferroptosis in a subject. In some embodiments, the agent is a small molecule, a peptide, or a nucleic acid. In some embodiments, the ferroptosis-modulating agent is an inhibitor of glutathione peroxidase 4 (GPX4), glutathione synthetase, glutamate-cysteine ligase, phosphoseryl-TRNA Kinase (PSTK), Eukaryotic Elongation Factor Selenocysteine-TRNA Specific (EEFSEC), Selenophosphate Synthetase 2 (SEPHS2), Sep (O-Phosphoserine) TRNA:Sec (Selenocysteine) TRNA Synthase (SEPSECS), or SECIS Binding Protein 2 (SECISBP2).

In some embodiments, the agent is an inhibitor of glutathione peroxidase 4 (GPX4). Glutathione peroxidase 4 (GPX4), also known as MCSP; SMDS; GPx-4; PHGPx; snGPx; GSHPx-4; snPHGPx, belongs to the glutathione peroxidase family, members of which catalyze the reduction of hydrogen peroxide, organic hydroperoxides and lipid hydroperoxides, and thereby protect cells against oxidative damage. GPX4 activation directly reduces phospholipid hydroperoxide levels in the cellular membrane. Several isozymes of this gene family exist in vertebrates, which vary in cellular location and substrate specificity. GPX4 has a high preference for lipid hydroperoxides and protects cells against membrane lipid peroxidation and cell death. This isozyme is also a selenoprotein, containing the rare amino acid selenocysteine (Sec) at its active site. Representative human GPX4 cDNA and human GPX4 protein sequences are publicly available from the National Center for Biotechnology Information (NCBI). Human glutathione peroxidase 4 peroxidase isoform B precursor (NM_001039847.3 and NP_001034936. 1), isoform C (NM_001039848.4 and NP_001034937.1), isoform D (NM_001367832.1 and NP_001354761.1), isoform A precursor (NM_002085.5 and NP_002076.2).

Depletion of GPX4 induces lipid peroxidation-dependent cell death. Cancer cells in a drug-induced, therapy-resistant state have an enhanced dependence on the lipid peroxidase activity of GPX4 to prevent undergoing ferroptotic cell death. Lipophilic antioxidants, such as ferrostatin, can rescue cells from GPX4 inhibition-induced ferroptosis. For instance, mesenchymal state GPX4-knockout cells can survive in the presence of ferrostatin, however, when the supply of ferrostatin is terminated, these cells undergo ferroptosis. GPX4 inhibition can be rescued by blocking other components of the ferroptosis pathways, such as lipid ROS scavengers (ferrostatin, liproxstatin), lipoxygenase inhibitors, iron chelators and caspase inhibitors, which an apoptotic inhibitor does not rescue. Accordingly, a GPX4 inhibitor can be useful to induce ferroptotic cell death.

In some embodiments, the agent is an inhibitor of glutathione synthetase (GSS). Glutathione synthetase (GSS), also known as GSHS; HEL-S-64p; HEL-S-88n is a homodimer to catalyze the second step of glutathione biosynthesis, which is the ATP-dependent conversion of gamma-L-glutamyl-L-cysteine to glutathione. Representative human GSS cDNA and human GSS protein sequences are publicly available from the National Center for Biotechnology Information (NCBI). Human glutathione synthetase (NM_000178.4 and NP_000169.1, NM_001322494.1 and NP_001309423.1, NM_001322495.1 and NP_001309424.1).

In some embodiments, the agent is an inhibitor of glutamate-cysteine ligase (GCL). Glutamate-cysteine ligase (GCL), a central node in the ferroptosis pathway, has been overlooked as a target. Loss of GCL activity induces ferroptosis in sensitive cells and kills only the most ferroptosis-sensitive cells. Representative human GCL cDNA and human GCL protein sequences are publicly available from the National Center for Biotechnology Information (NCBI). Human glutamate-cysteine ligase catalytic subunit isoform b (NM_001197115.2 and NP_001184044.1, which lacks an in-frame exon in the 5′ coding region, compared to variant 1. This results in a shorter protein (isoform b), compared to isoform a), and glutamate-cysteine ligase catalytic subunit isoform a (NM_001498.4 and NP_001489.1, which represents the longer transcript and encodes the longer isoform (a)).

In some embodiments, the agent is an inhibitor of phosphoseryl-TRNA Kinase (PSTK). PSTK is an enzyme that recruits selenocysteine, encoded by UGA. Sec is formed in a tRNA-dependent transformation of serine that is attached to tRNA Sec by seryl-tRNA synthetase. PSTK phosphorylates Ser-tRNA Sec to Sep-tRNA Sec which is then converted to Sec-tRNA Sec by Sep-ERNA:Sec-tRNA synthase (SepSecS). Representative human PSTK cDNA and human PSTK protein sequences are well-known in the art and are publicly available from the National Center for Biotechnology Information (NCBI). Human L-seryl-tRNA (Sec) kinase isoform 1 (NM_001363531.2 and NP_001350460.1), and L-seryl-tRNA (Sec) kinase isoform 2 (NM_153336.3 and NP_699167.2).

In some embodiments, the inhibitor is an inhibitor of Eukaryotic Elongation Factor Selenocysteine-TRNA Specific (EEFSEC). EEFSEC is also known as selenoprotein translation factor selb. Representative human EEFSEC cDNA and human EEFSEC protein sequences are publicly available from the National Center for Biotechnology Information (NCBI) as follows: selenocysteine-specific elongation factor (NM_021937.5 and NP_068756.2), selenocysteine-specific elongation factor isoform X4 (XM_024453695.1 and XP_024309463.1), selenocysteine-specific elongation factor isoform X3 (XM_024453694.1 and XP_024309462.1), selenocysteine-specific elongation factor isoform X1 (XM_024453692.1 and XP_024309460.1), selenocysteine-specific elongation factor isoform X2 (XM_024453693.1 and XP_024309461.1), selenocysteine-specific elongation factor isoform X5 (XM_005247696.3 and XP_005247753.1), selenocysteine-specific elongation factor isoform X7 (XM_011513066.2 and XP_011511368.1), and selenocysteine-specific elongation factor isoform X6 (XM_024453696.1 and XP_024309464.1). EEFSEC is a specialized translation elongation factor responsible for the co-translational incorporation of selenocysteine into proteins by recoding of a UGA stop codon in the presence of a downstream mRNA hairpin loop.

In some embodiments, the agent is an inhibitor of Selenophosphate Synthetase 2 (SEPHS2). Selenophosphate Synthetase 2 (SEPHS2) catalyzes the production of monoselenophosphate (MSP) from selenide and ATP. MSP is the selenium donor required for synthesis of selenocysteine (Sec), which is co-translationally incorporated into selenoproteins at in-frame UGA codons that normally signal translation termination. This protein is itself a selenoprotein containing a Sec residue at its active site, suggesting the existence of an autoregulatory mechanism. SEPHS2 is preferentially expressed in tissues implicated in the synthesis of selenoproteins and in sites of blood cell development. Further, genome-scale cancer-dependency profiling identifies selenoprotein synthesis enzymes as targets for ferroptosis induction. Loss of selenoprotein synthesis enzymes induces ferroptosis in sensitive cells. Moreover, Selenophosphate Synthetase 2 (SEPHS2) loss exhibits a novel two-pronged ferroptosis mechanism of action. SEPHS2 loss induced ferroptosis much more quickly than loss of other selenoprotein biosynthetic enzymes. SEPHS2 inhibitors can induce ferroptosis in certain diseases. For example, aggressive liver cancer is selectively targetable by SEPHS2 inhibition. Representative human SEPHS2 cDNA and human SEPHS2 protein sequences are well-known in the art and are publicly available from the National Center for Biotechnology Information (NCBI). Selenide, water dikinase 2 (NM_012248.4 and NP_036380.2).

In some embodiments, the agent is an inhibitor of Sep (O-Phosphoserine) TRNA:Sec (Selenocysteine) TRNA Synthase (SEPSECS). Sep (O-Phosphoserine) TRNA:Sec (Selenocysteine) TRNA Synthase (SEPSECS) catalyzes the third step in the process of selenocysteine synthesis, the conversion of O-phosphoseryl-tRNA (Sec) to selenocysteinyl-tRNA (Sec). Representative human SEPSECS cDNA and human SEPSECS protein sequences are publicly available from the National Center for Biotechnology Information (NCBI) as the follows: O-phosphoseryl-tRNA (Sec) selenium transferase (NM_016955.4 and NP_058651.3), O-phosphoseryl-tRNA (Sec) selenium transferase isoform X1 (XM_017008277.1 and XP_016863766.1), O-phosphoseryl-tRNA (Sec) selenium transferase isoform X5 (XM_017008278.1 and XP_016863767.1), O-phosphoseryl-tRNA (Sec) selenium transferase isoform X4 (XM_011513848.1 and XP_011512150.1), O-phosphoseryl-tRNA (Sec) selenium transferase isoform X2 (XM_011513846.2 and XP_011512148.1), and O-phosphoseryl-tRNA (Sec) selenium transferase isoform X3 (XM_011513847.2 and XP_011512149.1).

In some embodiments, the agent is an inhibitor of SECIS Binding Protein 2 (SECISBP2). SECISBP2 is one of the polypeptide components of the machinery involved in co-translational insertion of selenocysteine (Sec) into selenoproteins. Sec is encoded by the UGA codon, which normally signals translation termination. The recoding of UGA as Sec codon requires a Sec insertion sequence (SECIS) element; present in the 3′ untranslated regions of eukaryotic selenoprotein mRNAs. This protein specifically binds to the SECIS element, which is stimulated by a Sec-specific translation elongation factor. Representative human SECISBP2 cDNA and human SECISBP2 protein sequences are publicly available from the National Center for Biotechnology Information (NCBI) as follows: Selenocysteine insertion sequence-binding protein 2 isoform 2 (NM_001282688.2 and NP_001269617.1), selenocysteine insertion sequence-binding protein 2 isoform 3 (NM_001282689.2 and NP_001269618.1), selenocysteine insertion sequence-binding protein 2 isoform 4 (NM_001282690.1 and NP_001269619.1), selenocysteine insertion sequence-binding protein 2 isoform 5 (NM_001354696.2 and NP_001341625.1), selenocysteine insertion sequence-binding protein 2 isoform 6 (NM_001354697.2 and NP_001341626.1), selenocysteine insertion sequence-binding protein 2 isoform 7 (NM_001354698.2 and NP_001341627.1), selenocysteine insertion sequence-binding protein 2 isoform 8 (NM_001354702.2 and NP_001341631.1), selenocysteine insertion sequence-binding protein 2 isoform 1 (NM_024077.5 and NP_076982.3), selenocysteine insertion sequence-binding protein 2 isoform X9 (XM_024447669.1 and XP_024303437.1), selenocysteine insertion sequence-binding protein 2 isoform X8 (XM_024447667.1 and XP_024303435.1), selenocysteine insertion sequence-binding protein 2 isoform X5 (XM_017015122.2 and XP_016870611.1), selenocysteine insertion sequence-binding protein 2 isoform X6 (XM_024447666.1 and XP_024303434.1), selenocysteine insertion sequence-binding protein 2 isoform X9 (XM_024447668.1 and XP_024303436.1), selenocysteine insertion sequence-binding protein 2 isoform X1 (XM_011519000.2 and XP_011517302.1), selenocysteine insertion sequence-binding protein 2 isoform X2 (XM_011519001.1 and XP_011517303.1), selenocysteine insertion sequence-binding protein 2 isoform X3 (XM_011519002.1 and XP_011517304.1), selenocysteine insertion sequence-binding protein 2 isoform X4 (XM_011519003.1 and XP_011517305.1), and selenocysteine insertion sequence-binding protein 2 isoform X7 (XM_006717282.2 and XP_006717345.1).

In some embodiments, the agent is an inhibitor of Nuclear factor-erythroid factor 2-related factor 2 (NRF2). NRF2 is a member of the cap ‘n’ collar (CNC) subfamily of basic region leucine zipper (bZip) transcription factors. NRF2 mediates induction of a set of drug-metabolizing enzymes, such as glutathione S-transferase (GST) and NAD(P)H:quinone oxidoreductase 1 (NQO1), by antioxidants and electrophiles. NRF2 also regulates GPX4 protein content, intracellular free iron content, and mitochondrial function, thereby modulating ferroptosis. NRF2 protein sequences are publicly available from the National Center for Biotechnology Information (NCBI) as the follows: Nrf2 [Homo sapiens], GenBank: AAB32188.1; and transcription factor Nrf2—human, PIR: 159340.

In some embodiments, the agent is an inhibitor of cystine transporter SLC7A11 (also called xCT). SLC7A11 (also commonly known as xCT) functions to import cystine for glutathione biosynthesis and antioxidant defense and is overexpressed in multiple human cancers. SLC7A11 (xCT) protein sequences are publicly available from the National Center for Biotechnology Information (NCBI) as the follows: cystine/glutamate transporter [Homo sapiens] NP_055146.1, and cystine/glutamate transporter isoform X1 [Homo sapiens] XP_011530104.1.

In some embodiments, the agent is an inhibitor of system Xc. System Xc-, also named cystine/glutamate antiporter, is an intracellular antioxidant element composed of the light chain SLC7A11 (xCT) and the heavy chain SLC3A2 (4F2 hc) and functions as raw materials for the synthesis of glutathione (GSH). System Xc protein sequences are publicly available from the National Center for Biotechnology Information (NCBI) as the follows: cystine/glutamate transporter [Homo sapiens] NP_055146.1, cystine/glutamate transporter isoform X1 [Homo sapiens] XP_011530104.1, 4F2 cell-surface antigen heavy chain isoform f [Homo sapiens] NP_001013269.1, 4F2 cell-surface antigen heavy chain isoform c [Homo sapiens] NP_002385.3, 4F2 cell-surface antigen heavy chain isoform b [Homo sapiens] NP_001012680.1, and 4F2 cell-surface antigen heavy chain isoform e [Homo sapiens] NP_001012682.1.

In some embodiments, the agent is an inhibitor of thioredoxin reductase (TXNRD). TRXNRD is involved in reversible S-nitrosylation of cysteines in certain proteins. TRXNRD protein sequences are publicly available from the National Center for Biotechnology Information (NCBI) as the follows: thioredoxin reductase [Homo sapiens] AAB35418.1, thioredoxin reductase [Homo sapiens] AAF15900.1 GI: 6538774, thioredoxin reductase [Homo sapiens] AAD25167.1, thioredoxin reductase [Homo sapiens] AAD19597.1, and thioredoxin reductase [Homo sapiens] CAA04503.1.

In some embodiments, ferroptosis inducing agents are a class of molecules that include at least one of (for the molecules in the class): salts, pharmaceutically acceptable salts, solvates, hydrates, enantiomers, diastereomers, racemates, crystalline forms, or any combination of these, of the molecules.

In some embodiments, priming agents are a class of molecules that include at least one of (for the molecules in the class): salts, pharmaceutically acceptable salts, solvates, hydrates, enantiomers, diastereomers, racemates, crystalline forms, or any combination of these, of the molecules.

In some embodiments, the agent is a statin. Exemplary statins include but are not limited to: atorvastatin, cerivastatin, fluvastatin, lovastatin, mevastatin, pitavastatin, pravastatin, rosuvastatin and simvastatin.

In some embodiments, the agent that induces or modulates ferroptosis in a tissue or systemically is selected from Table 1. Exemplary ferroptosis-modulating agents are provided in Table 1 along with their formula, chemical identifiers, and respective target and/or mechanism of action.

TABLE 1
Ferroptosis-modulating agents

| | | | Target(s)/ |
|---|---|---|---|
| | Formula/ | | Mechanism of |
| Name | CAS | Structure | Action |
| (1S,3R)-RSL3 | C₂₃H₂₁ClN₂O₅ Cas: 1219810-16-8 | 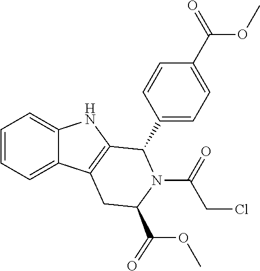 [SMILES: COC(=O)c1ccc([C@H]2c3[nH]c4ccccc4c3C[C@H](C(=O)OC)N2C(=O)CCl)cc1] | Ferroptosis inducer by GPX4 inhibition |
| altretamine | C₉H₁₈N₆ Cas: 645-05-6 | 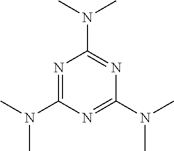 | Ferroptosis inducer by GPX4 inhibition |
| auranofin | C₂₀H₃₄AuO₉PS Cas: 34031-32-8 | 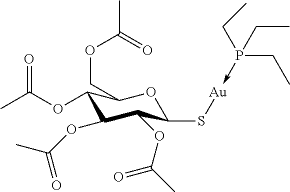 | Multiple modes of action including inhibition of thioredoxin reductase (TXNRD) |
| brusatol | C₂₆H₃₂O₁₁ Cas: 14907-98-3 | 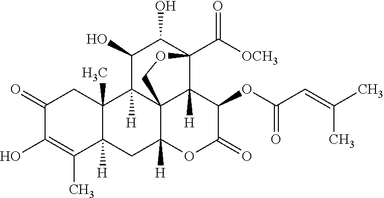 | Ferroptosis inducer by NRF2 inhibition. NRF2 is a transcriptional regulator of GPX4 protein content |
| Chloride[N,N′- disalicylidene- 1,2- phenylene- diamine] iron(III) | C₂₀H₁₄ClFeN₂O₂ Cas: 39916-28-4 | 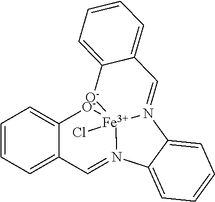 | Ferroptosis inducer by generating lipid- based reactive oxygen species (ROS) |
| CIL56 | C₂₃H₂₇N₃O₅S₂ Cas: 300802-282-2 | 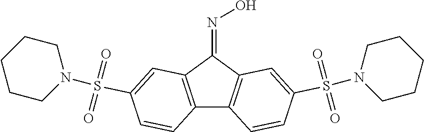 [SMILES: O=S(=O)(c1ccc2c(c1)C(=NO)c1cc(S(=O)(=O)N3CCCCC3)ccc1-2)N1CCCCC1] | Ferroptosis inducer by generating lipid- based reactive oxygen species (ROS) |
| Dihydroisotans- shinone I | C₁₈H₁₄O₃ Cas: 20958-18-3 | 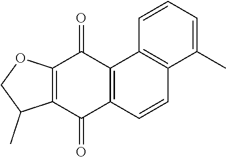 [SMILES: Cc1cccc2c3c(ccc12)C(=O)C1=C(OCC1C)C3=O] | Ferroptosis inducer by increasing lipid peroxidation and GPX4 inhibition |
| erastin | C₃₀H₃₁ClN₄O₄ Cas: 571203-78-6 | 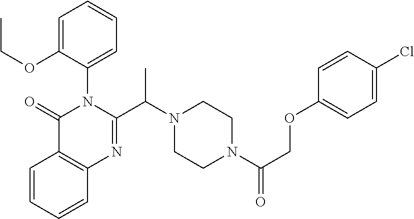 [SMILES: CCOc1ccccc1-n1c(C(C)N2CCN(C(=O)COc3ccc(Cl)cc3)CC2)nc2ccccc2c1=O] | Ferroptosis inducer by inhibition of cystine uptake by the system xc- cystine- glutamate transporter |
| Erastin-like-PE | C₃₅H₄₁ClN₆O₄ | 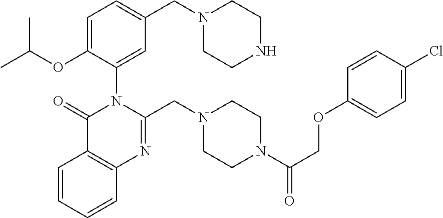 [SMILES: CC(C)Oc1ccc(CN2CCNCC2)cc1-n1c(CN2CCN(C(=O)COc3ccc(Cl)cc3)CC2)nc2ccccc2c1=O] | Ferroptosis inducer by inhibition of cystine uptake by the system xc- cystine- glutamate transporter |
| Erastin-like- IKE | C₃₅H₃₅ClN₆O₅ | 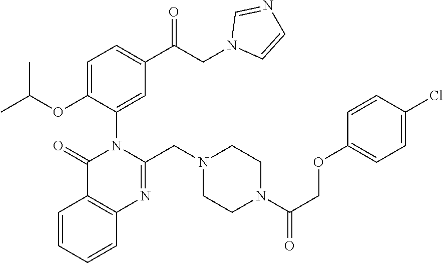 [SMILES: CC(C)Oc1ccc(C(=O)Cn2ccnc2)cc1-n1c(CN2CCN(C(=O)COc3ccc(Cl)cc3)CC2)nc2ccccc2c1=O] | Ferroptosis inducer by inhibition of cystine uptake by the system xc- cystine- glutamate transporter |
| erastin-related (Formula I) | | Formula I:, 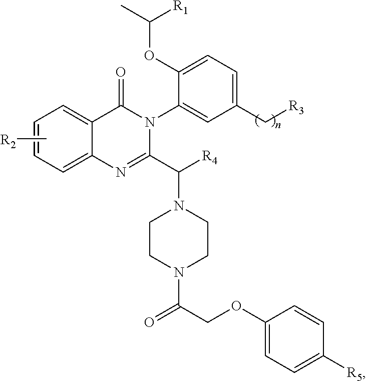 | Ferroptosis inducer by inhibition of cystine uptake by the system xc- glutamate transporter |
| | | or an enantiomer, optical isomer, diastereomer, N- | |
| | | oxide, crystalline form, hydrate, or pharmaceutically | |
| | | acceptable salt thereof, wherein | |
| | | wherein R₁ is selected from the group consisting of H, | |
| | | C₁₋₄alkyl, C₁₋₄alkoxy, hydroxy, and halogen; R₂ is | |
| | | selected from the group consisting of H, halo, and C1-4alkyl; | |
| | | R₃ is selected from the group consisting of H, | |
| | | C₁₋₄alkyl, C₁₋₄alkoxy, 5-7 membered | |
| | | heterocycloalkyl, and 5-6 membered heteroaryl; R₄ is | |
| | | selected from the group consisting of H and C₁₋₄alkyl; | |
| | | R₅ is halo; | |
| | | 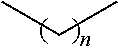 | |
| | | is optionally substituted with ═O; and | |
| | | n is an integer from 0-4. | |
| erastin-related (Formula II) | | Formula II: 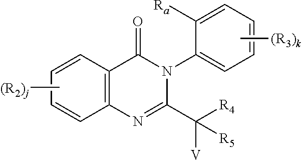 | Ferroptosis inducer by inhibition of cystine uptake by the system xc- cystine- glutamate transporter |
| | | or an enantiomer, optical isomer, diastereomer, N- | |
| | | oxide, crystalline form, hydrate, or pharmaceutically | |
| | | acceptable salt thereof, wherein | |
| | | Rₐ is a halogen, substituted or unsubstituted alkyl, | |
| | | substituted or unsubstituted alkenyl, substituted or | |
| | | unsubstituted alkynyl, substituted or unsubstituted | |
| | | aryl-O—, sustituted or unsubstituted alkyl-O—, | |
| | | substituted or unsubstituted alkenyl-O— or | |
| | | substituted or unsubstituted alkynyl-O—, where alkyl, | |
| | | alkenyl and alkynyl are optionally interrupted by NR, | |
| | | O or S(O)ₙ; each R₂ is independently selected from | |
| | | the group consisting of halogen, substituted or | |
| | | unsubstituted alkyl, substituted or unsubstituted aryl, | |
| | | substituted or unsubstituted non-aromatic | |
| | | heterocyclic, —CN, —COOR′, —CON(R)₂, | |
| | | —NRC(O)R, —SO₂N(R)₂, —N(R)₂, —NO₂, —OH and | |
| | | —OR′; each R₃ is independently selected from the | |
| | | group consisting of halogen, substituted or | |
| | | unsubstituted alkyl, substituted or unsubstituted aryl, | |
| | | substituted or unsubstituted non-aromatic | |
| | | heterocyclic, —C(O)R, —CN, —COOR′, | |
| | | —CON(R)₂, —NRC(O)R, —SO₂N(R)₂, —N(R)₂, | |
| | | —NO₂, —OH and —OR′; R₄ and R₅ are independently | |
| | | selected from the group consisting of —H, substituted | |
| | | or unsubstituted alkyl, substituted or unsubstituted | |
| | | alkenyl, substituted or unsubstituted alkynyl, | |
| | | substituted or unsubstituted non-aromatic heterocyclic | |
| | | and substituted or unsubstituted aryl, where alkyl, | |
| | | alkenyl and alkynyl are optionally interrupted by NR, | |
| | | O or (S)ₙ; or R₄ and R₅ taken together form a | |
| | | carbocyclic or heterocyclic group; V is | |
| | | 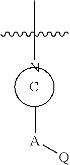 | |
| | | wherein Ring C is substituted or | |
| | | unsubstituted heterocyclic aromatic or non-aromatic | |
| | | ring; A is NR or O; or A is a covalent bond; L is a | |
| | | substituted or unsubstituted hydrocarbyl group | |
| | | optionally interrupted by one or more heteroatoms | |
| | | selected from N, O and S; Q is selected from the | |
| | | group consisting of —R, —C(O)R′, —C(O)N(R)2, | |
| | | —C(O)OR′, and —S(O)2R′; each R is independently | |
| | | —H, alkyl, alkenyl, alkynyl, aryl, or non-aromatic | |
| | | hetercyclic, wherein said alkyl, alkenyl, alkynyl, | |
| | | aryl, or non-aromatic heterocyclic groups are | |
| | | substituted or unsubstituted; each R′ is independently | |
| | | an alkyl, alkenyl, alkynyl group, non-aromatic | |
| | | heterocyclic or aryl group, wherein said alkyl, | |
| | | alkenyl, alkynyl, non-aromatic heterocyclic or aryl | |
| | | groups are substituted or unsubstituted; j is an integer | |
| | | from 0 to 4; k is an integer from 0 to 4, provided that | |
| | | at least one of j and k is an integer from 1 to 4; and | |
| | | each n is independently 0, 1 or 2. | |
| erastin-related (Formula III) | | Formula III: 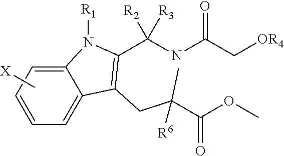 | Ferroptosis inducer by inhibition of cystine uptake by the system xc- cystine- glutamate transporter |
| | | or an enantiomer, optical isomer, diastereomer, N- | |
| | | oxide, crystalline form, hydrate, or pharmaceutically | |
| | | acceptable salt thereof, wherein R₁, R₂, R₃, and R₆ are | |
| | | independently selected from H, C₁₋₈alkyl, C₁₋₈alkoxy, | |
| | | C₁₋₈aralkyl, 3- to 8-membered carbocyclic, 3- to 8- | |
| | | membered heterocyclic, 3- to 8-membered aryl, or 3- | |
| | | to 8-membered heteroaryl, acyl, alkylsulfonyl, and | |
| | | arylsulfonyl, wherein each alkyl, alkoxy, aralkyl, | |
| | | carbocyclic, heterocyclic, aryl, heteroaryl, acyl, | |
| | | alkylsulfonyl, and arylsulfonyl is optionally | |
| | | substituted with at least one substituted; R₄ and R₅ are | |
| | | independently selected from H1 C₁₋₈alkyl, | |
| | | C₁₋₈alkoxy, 3- to 8-membered carbocyclic, 3- to 8- | |
| | | membered heterocyclic, 3- to 8-membered aryl, or 3- | |
| | | to 8-membered heteroaryl, carboxylate, ester, amide, | |
| | | carbohydrate, amino acid, acyl, alkoxy-substituted | |
| | | acyl, alditol, NR⁷R⁸, OC(R⁷)₂COOH, SC(R⁷)₂COOH, | |
| | | NHCHR⁷COOH, COR⁸, CO₂R₈, sulfate, sulfonamide, | |
| | | sulfoxide, sulfonate, sulfone, thioalkyl, thioester, and | |
| | | thioether, wherein each alkyl, alkoxy, carbocyclic, | |
| | | heterocyclic, aryl, heteroaryl, carboxylate, ester, | |
| | | amide, carbohydrate, amino acid, acyl, alkoxy- | |
| | | substituted acyl, alditol, NR⁷R⁸, OC(R⁷)₂COOH, | |
| | | SC(R⁷)₂COOH, NHCHR⁷COOH, COR⁸, CO₂R⁸, | |
| | | sulfate, sulfonamide, sulfoxide, sulfonate, sulfone, | |
| | | thioalkyl, thioester, and thioether is optionally | |
| | | substituted with at least one substituent; R₇ is selected | |
| | | from H, C₁₋₈alkyl, carbocycle, aryl, heteroaryl, | |
| | | heterocycle, alkylaryl, alkylheteroaryl, and | |
| | | alkylheterocycle, wherein each alkyl, carbocycle, aryl, | |
| | | heteroaryl, heterocycle, alkylaryl, alkylheteroaryl, and | |
| | | alkylheterocycle may be optionally substituted with at | |
| | | least one substituent; R₈ is selected from H, C₁₋₈alkyl, | |
| | | C₁₋₈alkenyl, C₁₋₈alkynyl, aryl, carbocycle, heteroaryl, | |
| | | heterocycle, alkylaryl, alkylheteroaryl, | |
| | | alkylheterocycle, and heteroaromatic, wherein each | |
| | | alkyl, alkenyl, alkynyl, aryl, carbocycle, heteroaryl, | |
| | | heterocycle, alkylaryl, alkylheteroaryl, | |
| | | alkylheterocycle, and heteroaromatic may be | |
| | | optionally substituted with at least one substituent; | |
| | | and X is 0-4 substituents on the ring to which it is | |
| | | attached. | |
| erastin-related (Formula IV) | | Formula IV: 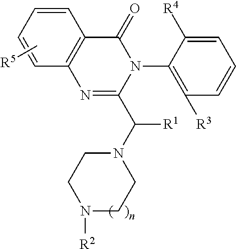 | Ferroptosis inducer by inhibition of cystine uptake by the system xc- cystine- glutamate transporter |
| | | or an enantiomer, optical isomer, diastereomer, N- | |
| | | oxide, crystalline form, hydrate, or pharmaceutically | |
| | | acceptable salt thereof, wherein | |
| | | R₁ is selected from H and C₁₋₈alkyl; R₂ is selected | |
| | | from H and C₁₋₈alkyl; R₃ is selected from halogen, | |
| | | C₁₋₈alkoxy and C₁₋₈alkyl; R₄ is selected from H, halogen, | |
| | | C₁₋₈alkoxy and C₁₋₈alkyl; R₅ is selected from H, | |
| | | halogen and nitro; and n is 1 or 2. | |
| erastin-related (Formula V) | | Formula V: 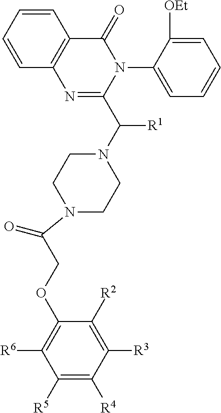 | Ferroptosis inducer by inhibition of cytsine uptake by the system xc- cystine- glutamate transporter |
| | | wherein, R₁ is selected from H, C₁₋₈alkyl, C₁₋₈alkoxy, | |
| | | 3- to 8-membered carbocyclic or heterocyclic, aryl, | |
| | | heteroaryl, C₁₋₄aralkyl, residues of glycolic acid, | |
| | | ethylene glycol/proplylene glycol copolymers, | |
| | | carboxylate, ester, amide, carbohydrate, amino acid, | |
| | | alditol, OC(R⁷)₂COOH, SC(R⁷)₂COOH, | |
| | | NHCHR⁷COOH, COR⁸, CO²R⁸, sulfate, sulfonamide, | |
| | | sulfoxide, sulfonate, sulfone, thioalkyl, thioester, and | |
| | | thioether, R₂, R₃, R₄, R₅, and R₆ are independently | |
| | | selected from H, halo, C₁₋₄alkyl, C₁₋₄alkylamino, acyl, | |
| | | and alkylsulfonyl; R₇ is selected from H, C₁₋₈alkyl, | |
| | | optionally substituted carbocycle, aryl, heteroaryl, | |
| | | heterocycle, alkylaryl, alkylheteroaryl, and | |
| | | alkylheterocycle; and R₈ is selected from optionally | |
| | | substituted C₁₋₈alkyl, C₁₋₈alkenyl, C₁₋₈alkynyl, aryl, | |
| | | carbocycle, heteroaryl, heterocycle, alkylaryl, | |
| | | alkylheteroaryl, alkylheterocycle, and heteroaromatic. | |
| erastin-like-AM | | 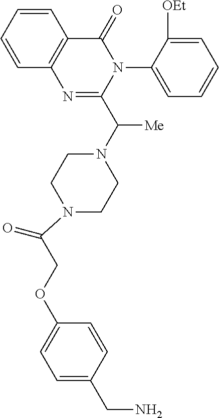 [SMILES: CCOc1ccccc1-n1c(C(C)N2CCN(C(=O)COc3ccc(CN)cc3)CC2)nc2ccccc2c1=O] | Ferroptosis inducer by inhibition of cystine uptake by the system xc- cystine- glutamate transporter |
| erastin-like- PHTL | | 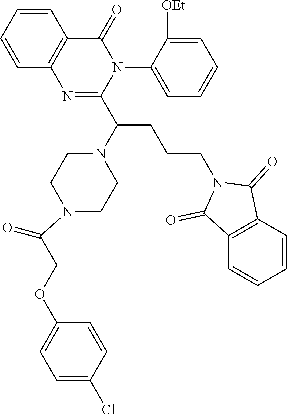 [SMILES: CCOc1ccccc1-n1c(C(CCCN2C(=O)c3ccccc3C2=O)N2CCN(C(=O)COc3ccc(Cl)cc3)CC2)nc2ccccc2c1=O] | Ferroptosis inducer by inhibition of cystine uptake by the system xc- cystine- glutamate transporter |
| erastin-like-H2 | | 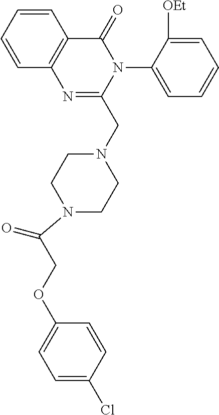 [SMILES: CCOc1ccccc1-n1c(CN2CCN(C(=O)COc3ccc(Cl)cc3)CC2)nc2ccccc2c1=O] | Ferroptosis inducer by inhibition of cystine uptake by the system xc- cystine- glutamate transporter |
| erastin-related (Formula VI) | | Formula VI: 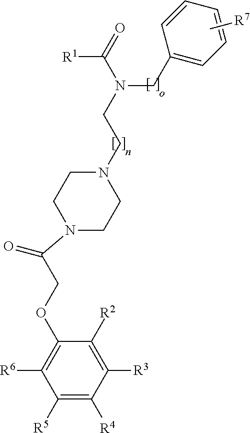 | Ferroptosis inducer by inhibition of cystine uptake by the system xc- cystine- glutamate transporter |
| | | or an enantiomer, optical isomer, diastereomer, N- | |
| | | oxide, crystalling form, hydrate, or pharmaceutically | |
| | | acceptable salt thereof, | |
| | | wherein R₁ is selected from C₁₋₈alkyl, C₁₋₈alkyl-OR³, | |
| | | 3- to 8-membered carbocyclic or heterocyclic, aryl, | |
| | | heteroaryl, C₁₋₄aralkyl, nitrogen substituted with | |
| | | C₁₋₆alkyl, hydroxy substituted C₁₋₆alkyl, and C₁₋₄alkoxy; | |
| | | R₂, R₃, R₄, R₅, and R₆ are independently selected from | |
| | | H, halo, C₁₋₄alkyl, C₁₋₄alkylamino, acyl, and | |
| | | alkylsulfonyl; R₇ is selected from halo, C₁₋₈alkyl, | |
| | | C₁₋₈alkylamino, C₁₋₈alkylthio, C₁₋₈alkoxy, C₁₋₈alkynyl, | |
| | | amide, amine, carbamate, carbonate, carboxy, acyl, | |
| | | ether, heteroalkyl, and aralkyl; and n and o are | |
| | | independently selected from an integer from 1 to 4. | |
| erastin-related (Formula VII) | | Formula VII: 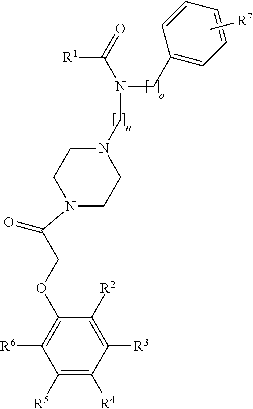 | Ferroptosis inducer by inhibition of cystine uptake by the system xc- glutamate transporter |
| | | or an enatiomer, optical isomer, diastereomer, N- | |
| | | oxide, crystalline form hydrate, or pharmaceutically | |
| | | acceptable salt thereof, | |
| | | wherein R₁ is selected from methyl, ethyl, propyl, | |
| | | phenyl, and a substituted N; R₂, R₃, R₄, R₅, and R₆ are | |
| | | independently selected from H, halo, C₁₋₄alkyl, | |
| | | C₁₋₄alkylamino, acyl, and alkylsulfonyl R₇ is F; n is 2; o is 1 | |
| RSL-like compound (Formula VIII) | | Formula VIII: 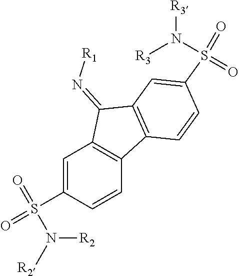 | Ferroptosis inducer by inhibition of cystine uptake by the system xc- cystine- glutamate transporter |
| | | or an N-oxide, crystalline form, hydrate, or | |
| | | pharmaceutically acceptable salt thereof; wherein: R₁ | |
| | | is selected from the group consisting of H, OH, and | |
| | | (—OCH₂CH₂)ₓOH; X is an integer from 1 to 6; and R₂, | |
| | | R_{2′}, R₃, R_{3′} independently are selected from the | |
| | | group consisting of H, C₃₋₈cycloalkyl, and | |
| | | combinations thereof, or R₂ and R_{2′} may be joined | |
| | | together to form a pyridinyl or pyranyl and R₃ and R_{3′} | |
| | | may be joined together to form a pyridinyl or pyranyl; | |
| | | or an N-oxide, or pharmaceutically acceptable salt | |
| | | thereof. | |
| RSL-like compound (Formula IX) | | Formula IX: 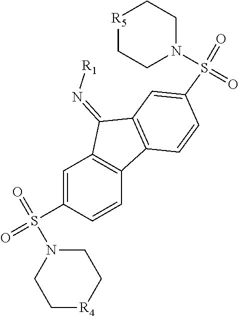 | Ferroptosis inducer by GPX4 inhibition |
| | | R₁ is selected from the group consisting of OH and | |
| | | —(OCH₂CH₂)ₓOH; X is an integer from 1 to 6; and R₄ | |
| | | and R₅ are independently selected from the group | |
| | | consisting of CH₂ and O; or an N-oxide; or | |
| | | wherein: R₁ is OH and (1) R₄ and R₅ are both O; or | |
| | | (2) R₄ is CH₂ and R₅ is O; or (3) R₄ is O and R₅ is CH₂ | |
| | | or an N-oxide, or pharmaceutically acceptable salt | |
| | | thereof. | |
| RSL-like compound SRS8-18 | | 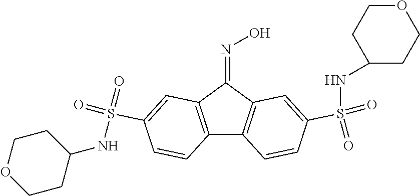 [SMILES: O=S(=O)(NC1CCOCC1)c1ccc2c(c1)C(=NO)c1cc(S(=O)(=O)NC3CCOCC3)ccc1-2] | Ferroptosis inducer by GPX4 inhibition |
| | | SRS8-18 | |
| RSL-like compound SRS11-31 | | 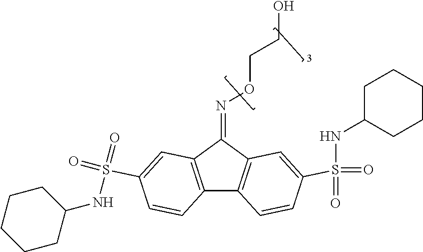 | Ferroptosis inducer by GPX4 inhibition |
| | | SRS11-31 | |
| RSL-like compound SRS11-66 | | 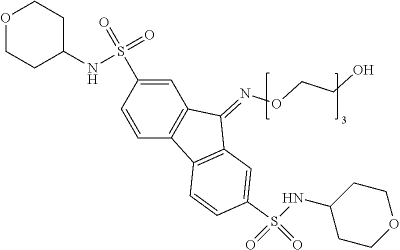 | Ferroptosis inducer by GPX4 inhibition |
| | | SRS11-66 | |
| Ferroptosis inducer (Formula X) | | Formula X: 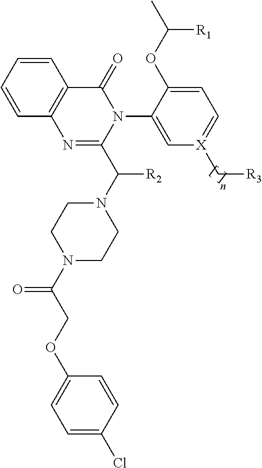 | Ferroptosis inducer by GSH inhibition |
| | | wherein R₁ is selected from the group consisting of H, | |
| | | C₁₋₄alkyl, C₁₋₄alkoxy, hydroxy, and halogen; R₂ is | |
| | | selected from the group consisting of H, C₁₋₄ alkyl, | |
| | | C₁₋₄alkoxy, C₃₋₈cycloalkyl, C₃₋₈heterocycloalkyl, aryl, | |
| | | heteroaryl, C₁₋₄aralkyl; R₃ is selected from the group | |
| | | consisting of nothing, C₁₋₄alkyl, C₁₋₄alkoxy, carbonyl, | |
| | | C₃₋₈cycloalkyl, and C₃₋₈heterocycloalkyl; X is | |
| | | selected from the group consisting of C, N, and O; | |
| | | and n is an integer from 0-6, with the proviso that | |
| | | when X is C, n = 0, and R₃ is nothing, R₁ cannot be H | |
| | | when R₂ is CH₃, or an N-oxidecrystalline form, | |
| | | hydrate, or pharmaceutically acceptable salt thereof. | |
| Ferroptosis Inducer (Formula XI) | | Formula XI: 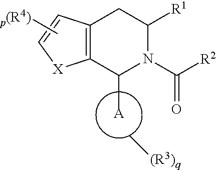 | Ferroptosis inducer by GPX4 inhibition |
| | | wherein: ring A is C₄₋₁₀cycloalkyl, heterocyclyl, | |
| | | aryl, or heteroaryl; X is —O—, —S—, —NR₉—, | |
| | | —CR⁵═CR⁵—, or —CR⁵═N—; p is 0, 1 or 2; q is 0, 1, 2 | |
| | | or 3; R₁ is C₁₋₆alkyl, C₂-C₆alkenyl, C₂-C₆alkynyl, | |
| | | C₁-C₆haloalkyl, C₃-C₁₀cycloalkyl, —CN, —OR⁷, | |
| | | —C(O)OR⁶, —C(O)N(R⁷)₂, —OC(O)R⁶, —S(O)₂R⁸, | |
| | | —S(O)₂N(R⁷)₂, —S(O)N(R⁷)₂, —S(O)R⁸, —N(R⁷)₂, | |
| | | —NO₂, —C₁-C₆alkyl —OR⁷, or —Si(R¹⁵)₃; R₂ is | |
| | | —C₁-C₂haloalkyl, —C₂-C₃alkenyl, —C₂-C₃haloalkenyl, | |
| | | C₂alkynyl, or —CH₂OS(O)₂-phenyl, wherein the | |
| | | C₁-C₂alkylhalo and —C₂-C₃alkenylhalo are optionally | |
| | | substituted with one or two —CH₃, and the C₂alkynyl | |
| | | and phenyl are optionally substituted with one —CH₃; | |
| | | each R₃ is independently halo, —CN, —OH, —OR⁸, | |
| | | —NH₂, —NHR⁸, —N(R⁸)₂, —S(O)₂R⁸, —S(O)R⁸, | |
| | | —S(O)₂N(R⁷)₂, —S(O)N(R⁷)₂, —NO₂, —Si(R¹²)₃, | |
| | | —SF₅, —C(O)OR⁶, —C(O)N(R⁷)₂, —NR¹²C(O)R₈, | |
| | | —NR¹²C(O)OR⁸, —OC(O)N(R⁷)₂, —OC(O)R⁸, | |
| | | —C(O)R⁶, —OC(O)CHR⁸N(R¹²)₂, C₁-C₆alkyl, | |
| | | C₂-C₆alkenyl, C₂-C₆alkynyl, C₃-C₁₀cycloalkyl, | |
| | | heterocyclyl, aryl, heteroaryl, —C₁-C₆alkylC₃-C₁₀cycloalkyl, | |
| | | —C₂-C₆alkenylC₃-C₁₀cycloalkyl, | |
| | | —C₁-C₆alkylheterocyclyl, —C₂-C₆alkenylheterocyclyl, | |
| | | —C₁-C₆alkylaryl, —C₂-C₆alkenylaryl, | |
| | | C₁-C₆alkylheteroaryl, or —C₂-C₆alkenylheteroaryl; | |
| | | wherein each C₁-C₆alkyl, C₂-C₆alkenyl, C₂-C₆alkynyl, | |
| | | C₃-C₁₀cycloalkyl, heterocyclyl, aryl, heteroaryl, —C₁-C₆alkyl | |
| | | C₃-C₁₀cycloalkyl, —C₂-C₆alkenylC₃-C₁₀cycloalkyl, | |
| | | —C₁-C₆alkylheterocyclyl, —C₂-C₆alkenylheterocyclyl, | |
| | | —C₁-C₆alkylaryl, —C₂-C₆alkenylaryl, | |
| | | —C₁-C₆alkylheteroaryl, or | |
| | | —C₂-C₆alkenylheteroaryl of R₃ is independently optionally | |
| | | substituted with one to three R₁₀; each R⁴ is | |
| | | independently halo, —CN, —OH, —OR⁸, —NH₂, | |
| | | —NHR⁸, —N(R⁸)₂, —S(O)₂R⁸, —S(O)R⁸, | |
| | | —S(O)₂N(R⁷)₂, —S(O)N(R⁷)₂, —NO₂, —Si(R⁵)₃, | |
| | | —C(O)OR⁶, —C(O)N(R⁷)₂, —NR¹²C(O)R⁸, —OC(O)R, | |
| | | —C(O)R⁶, —NR¹²C(O)OR⁸, —OC(O)N(R⁷)₂, | |
| | | —OC(O)CHR⁸N(R¹²)₂, C₁-C₆alkyl, C₂-C₆alkenyl, | |
| | | C₂-C₆alkynyl, C₃-C₁₀cycloalkyl, heterocyclyl, aryl, | |
| | | heteroaryl, —C₁-C₆alkylC₃-C₁₀cycloalkyl, | |
| | | —C₂-C₆alkenylC₃-C₁₀cycloalkyl, —C₁-C₆alkylheterocyclyl, | |
| | | —C₂-C₆alkenylheterocyclyl, —C₁-C₆alkylaryl, | |
| | | —C₂-C₆alkenylaryl, C₁-C₆alkylheteroaryl, or | |
| | | —C₂-C₆alkenylheteroaryl; wherein each C₁-C₆alkyl, | |
| | | C₂-C₆alkenyl, C₂-C₆alkynyl, C₃-C₁₀cycloalkyl, | |
| | | heterocyclyl, aryl, heteroaryl, —C₁-C₆alkylC₃-C₁₀cycloalkyl, | |
| | | —C₂-C₆alkenylC₃-C₁₀cycloalkyl, | |
| | | —C₁-C₆alkylheterocyclyl, —C₂-C₆alkenylheterocyclyl, | |
| | | —C₁-C₆alkylaryl, —C₂-C₆alkenylaryl, | |
| | | C₁-C₆alkylheteroaryl, or —C₂-C₆alkenylheteroaryl of R⁴ is | |
| | | optionally independently optionally substituted with | |
| | | one to three R¹⁰; | |
| | | each R⁵ is independently hydrogen, halo, —CN, | |
| | | —OH, —OR⁸, —NH₂, —NHR⁸, —N(R⁸)₂, —S(O)₂R⁸, | |
| | | —S(O)R⁸, —S(O)₂N(R⁷)₂, —S(O)N(R⁷)₂, —NO₂, | |
| | | —Si(R⁵)₃, —C(O)OR⁶, —C(O)N(R⁷)₂, —NR¹²C(O)R⁸, | |
| | | —OC(O)R⁸, —C(O)R⁶, —NR¹²C(O)OR⁸, | |
| | | —OC(O)N(R⁷)₂, —OC(O)CHR⁸N(R¹²)₂, C₁-C₆alkyl, | |
| | | C₂-C₆alkenyl, C₂-C₆alkynyl, C₃-C₁₀cycloalkyl, | |
| | | heterocyclyl, aryl, heteroaryl, —C₁-C₆alkylC₃-C₁₀cycloalkyl, | |
| | | —C₂-C₆alkenylC₃-C₁₀cycloalkyl, | |
| | | —C₁-C₆alkylheterocyclyl, —C₂-C₆alkenylheterocyclyl, | |
| | | —C₁-C₆alkylaryl, —C₂-C₆alkenylaryl, | |
| | | C₁-C₆alkylheteroaryl, or —C₂-C₆alkenylheteroaryl, | |
| | | wherein each C₁-C₆alkyl, C₂-C₆alkenyl, C₂-C₆alkynyl, | |
| | | C₃-C₁₀cycloalkyl, heterocyclyl, aryl, heteroaryl, | |
| | | —C₁-C₆alkylC₃-C₁₀cycloalkyl, —C₂-C₆alkenylC₃-C₁₀cycloalkyl, | |
| | | —C₁-C₆alkylheterocyclyl, —C₂-C₆alkenylheterocyclyl, | |
| | | —C₁-C₆alkylaryl, —C₂-C₆alkenylaryl, | |
| | | C₁-C₆alkylheteroaryl, or —C₂-C₆alkenylheteroaryl | |
| | | of R is optionally independently | |
| | | optionally substituted with one to three R¹⁰; | |
| | | each R⁶ is independently hydrogen, C₁-C₆alkyl, | |
| | | C₂-C₆alkenyl, C₂-C₆alkynyl, C₃-C₁₀cycloalkyl, | |
| | | heterocyclyl, aryl, heteroaryl, —C₁-C₆alkylC₃-C₁₀cycloalkyl, | |
| | | —C₂-C₆alkenylC₃-C₁₀cycloalkyl, | |
| | | —C₁-C₆alkylheterocyclyl, —C₂-C₆alkenylheterocyclyl, | |
| | | —C₁-C₆alkylaryl, —C₂-C₆alkenylaryl, | |
| | | C₁-C₆alkylheteroaryl, or —C₂-C₆alkenylheteroaryl; | |
| | | wherein each R⁶ is independently further substituted | |
| | | with one to three R¹¹; | |
| | | each R⁷ is independently hydrogen, C₁-C₆alkyl, | |
| | | C₂-C₆alkenyl, C₂-C₆alkynyl, C₃-C₁₀cycloalkyl, | |
| | | heterocyclyl, aryl, heteroaryl, —C₃-C₆alkylC₃-C₆cycloalkyl, | |
| | | —C₂-C₆alkenylC₃-C₆cycloalkyl, | |
| | | —C₁-C₆alkylheterocyclyl, —C₂-C₆alkenylheterocyclyl, | |
| | | —C₁-C₆alkylaryl, —C₂-C₆alkenylaryl, | |
| | | —C₃-C₆alkylheteroaryl, —C₂-C₆alkenylheteroaryl, or two | |
| | | R⁷ together with the nitrogen atom to which they are | |
| | | attached, form a 4 to 7 membered heterocyclyl; | |
| | | wherein each R⁷ or ring formed thereby is | |
| | | independently further substituted with one to three R¹¹; | |
| | | each R⁸ is independently C₁-C₆alkyl, C₂-C₆alkenyl, | |
| | | C₂-C₆alkynyl, C₃-C₁₀cycloalkyl, heterocyclyl, aryl, | |
| | | heteroaryl, —C₁-C₆alkylC₃-C₁₀cycloalkyl, | |
| | | —C₂-C₆alkenylC₃-C₁₀cycloalkyl, —C₁-C₆alkylheterocyclyl, | |
| | | —C₂-C₆alkenylheterocyclyl, —C₁-C₆alkylaryl, | |
| | | —C₂-C₆alkenylaryl, —C₁-C₆alkylheteroaryl, or | |
| | | —C₂-C₆alkenylheteroaryl; wherein each R⁸ is independently | |
| | | further substituted with one to three R¹¹; | |
| | | R⁹ is hydrogen or C₁-C₆alkyl; | |
| | | each R¹⁰ is independently halo, —CN, —OR¹², | |
| | | —NO₂, —N(R¹²)₂, —S(O)R³, —S(O)₂R¹³, | |
| | | —S(O)N(R¹²)₂, —S(O)₂N(R¹²)₂, —Si(R¹²)_{3,} —C(O)R¹², | |
| | | —C(O)OR¹², —C(O)N(R¹²)₂, —NR¹²C(O)R¹², | |
| | | —OC(O)R¹², —OC(O)OR¹², —OC(O)N(R¹²)₂, | |
| | | —NR¹²C(O)OR¹², —OC(O)CHR¹²N(R¹²)₂, C₁-C₆alkyl, | |
| | | C₁-C₆haloalkyl, C₂-C₆alkenyl, C₂-C₆alkynyl, | |
| | | C₃-C₁₀cycloalkyl, heterocyclyl, aryl, or heteroaryl, | |
| | | wherein each C₁-C₆alkyl, C₁-C₆haloalkyl, | |
| | | C₂-C₆alkenyl, C₂-C₆alkynyl, C₃-C₁₀cycloalkyl, | |
| | | heterocyclyl, aryl, or heteroaryl of R¹⁰ is optionally | |
| | | independently substituted with one to three R¹¹; | |
| | | each R¹¹ is independently halo, —CN, —OR¹², | |
| | | —NO₂, —N(R¹²)₂, —S(O)R³, —S(O)₂R¹³, | |
| | | —S(O)N(R¹²)₂, —S(O)₂N(R¹²)₂, —Si(R¹²)₃, —C(O)R¹², | |
| | | —C(O)OR¹², —C(O)N(R¹²)₂, —NR¹²C(O)R¹², | |
| | | —OC(O)R¹², —OC(O)OR¹², —OC(O)N(R¹²)₂, | |
| | | —NR¹²C(O)OR¹², —OC(O)CHR¹²N(R¹²)₂, C₁-C₆alkyl, | |
| | | C₁-C₆haloalkyl, C₂-C₆alkenyl, C₂-C₆alkynyl, | |
| | | C₃-C₁₀cycloalkyl, heterocyclyl, aryl, or heteroaryl; | |
| | | each R¹² is independently hydrogen, C₁-C₆alkyl or | |
| | | C₃-C₁₀cycloalkyl; each R¹³ is independently C₁-C₆alkyl or | |
| | | C₃-C₁₀cycloalkyl; and | |
| | | each R¹⁵ is independently C₁-C₆alkyl, C₂-C₆alkenyl, | |
| | | aryl, heteroaryl, —C₁-C₆alkylaryl, —C₂-C₆alkenylaryl, | |
| | | —C₁-C₆alkylheteroaryl, and | |
| | | —C₂-C₆alkenylheteroaryl; provided that at least one of the | |
| | | following is true: | |
| | | 1) R¹ is other than —C(O)OCH₃; | |
| | | 2) R² is —C₂alkynyl optionally substituted with one | |
| | | —CH₃; or | |
| | | 3) when R¹ is —C(O)OCH₃ and R² is —CH₂Cl, then the moiety | |
| | | 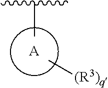 | |
| | | is other than 1,3-benzodioxol-5-yl, 4-nitrophenyl, 4- | |
| | | bromophenyl, cyclohexyl, furyl, or 4-methoxyphenyl. | |
| Ferroptosis Inducer (Formula XII) | | Formula XII: 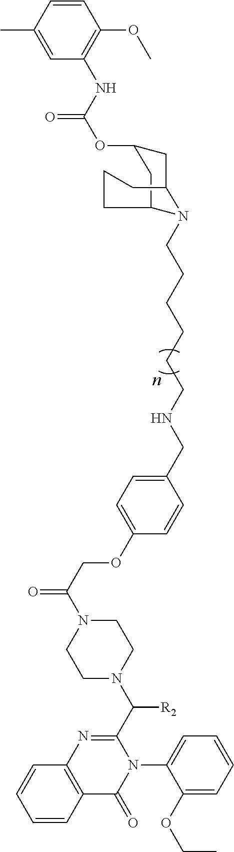 | Induces ferroptosis by binding the Sigma-2 receptor and having GPX4 inhibitor activity. |
| | | or an enantiomer, optical isomer, diastereomer, N- | |
| | | oxide, crystalline form, hydrate, or pharmaceutically | |
| | | acceptable salt thereof, wherein n is an integer chosen | |
| | | from 1, 2, 3, 4, 5, and R₂ is H, CH₃. | |
| Ferroptosis Inducer (Formula XIII) | | Formula III: 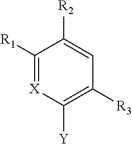 | Ferroptosis inducer by reducing reactive oxygen species (ROS) in a cell |
| | | or an enantiomer, optical isomer, diastereomer, N-oxide, | |
| | | crystalline form, hydrate, or pharmaceutically acceptable salt, thereof, | |
| | | wherein X is N; Y is H, halo, or C₁₋₄alkyl; R₁ is NR₄R₅, and at least one of | |
| | | R₄ and R₅ has a ring structure as defined below; R₂ is NR₆R₇; | |
| | | R₃ is selected from the group consisting of H, | |
| | | 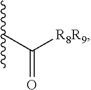 and | |
| | | 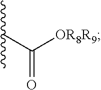 | |
| | | R₄ and R₅ are independently selected from the group consisting of H, | |
| | | C₁₋₁₂alkyl, C₃₋₁₂cycloalkyl, and aryl, | |
| | | wherein one or more of the ring carbons of the cycloalkyl are optionally | |
| | | substituted with one or more heteroatoms, and cycloalkyl optionally comprises | |
| | | one of more pendant groups selected from the group | |
| | | consisting of H, F, NR₁₀R₁₁, Boc, COOR₁₂, and C₁₋₈alkyl; | |
| | | R₆ and R₇ are independently selected from the group consisting of H, | |
| | | C₁₋₆alkyl, Boc, O, COOR₁₂, | |
| | | 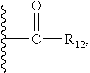 | |
| | | and C₁₋₃alkyl-aryl, wherein one or more of the ring | |
| | | carbprons of the alkyl-aryl are | |
| | | optionally substituted with one or more nitrogen atoms, | |
| | | and the alkyl-aryl optionally comprises one or more | |
| | | pendant groups selected from the group consisting of H, halo, | |
| | | CN, NO₂, C₁₋₄ester, C₁₋₄ester, OCOOR₁₂, and | |
| | | C₁₋₈alkyl, which C₁₋₈alkyl is optionally further substituted | |
| | | with one or more halo; R₈ and R₉ are | |
| | | independently selected from the group consisting of no atom, O, | |
| | | N, NHR₁₂, C₁₋₁₀alkyl, and C₁₋₁₀ether, wherein | |
| | | the alkyl and the ether are optionally substituted with NH₂, NHBoc, | |
| | | or C₃₋₁₂cycloalkyl, wherein one or more | |
| | | of the ring carbons of the cycloalkyl are optionally substituted | |
| | | with one or more heteroatoms; R₁₀ and R₁₁ | |
| | | are independently selected from H and Boc; and R₁₂ is a C₁₋₄alkyl | |
| | | optionally substituted with aryl. | |
| Ferroptosis Modulator X | | 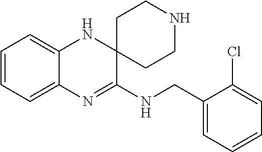 [SMILES: Clc1ccccc1CNC1=Nc2ccccc2NC12CCNCC2] | Modulates ferroptosis by altering reactive oxygen species (ROS) |
| Ferroptosis Modulator Y | | 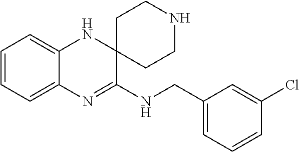 [SMILES: Clc1cccc(CNC2=Nc3ccccc3NC23CCNCC3)c1] | Modulates ferroptosis by altering reactive oxygen species (ROS) |
| FIN56 | C₂₅H₃₁N₃O₅S₂ Cas: 1083162-61-1 | 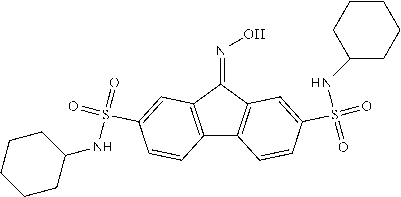 [SMILES: O=S(=O)(NC1CCCCC1)c1ccc2c(c1)C(=NO)c1cc(S(=O)(=O)NC3CCCCC3)ccc1-2] | Induces ferroptosis by: (1) promoting the degradation of GPX4 and (2) reducing the abundance of CoQ10 (i.e., an antioxidant in the cell) |
| FINO₂ | C₁₅H₂₈O₃ Cas: 869298-31-7 | 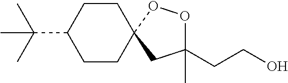 | Induces ferroptosis by increasing lipid peroxidation and inhibiting GPX4 activity |
| glutamate | C₅H₉NO₄ Cas: 139883-82-2 | 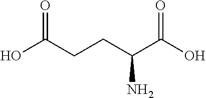 | High extracellular glutamate concentrations prevent cystine import, causes GSH depletion |
| | | | increasing |
| | | | ferroptosis |
| | | | sensitivity and |
| | | | induction |
| GPX4-IN-3 | C₂₉H₂₄ClN₃O₃S | 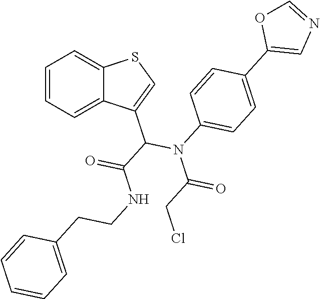 [SMILES: O=C(NCCc1ccccc1)C(c1csc2ccccc12)N(C(=O)CCl)c1ccc(-c2cnco2)cc1] | Ferroptosis inducer by GPX4 inhibition |
| jacaric acid | C₁₈H₃₀O₂ Cas: 28872-28-8 | 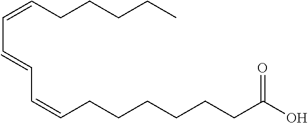 [SMILES: CCCCC\C=C/C=C/C=C\CCCCCCC(=O)O] | Induces ferroptosis by modulating the production of reactive oxygen species |
| JKE-1674 | C₂₀H₂₀Cl₂N₄O₄ Cas: 2421119-60-8 | 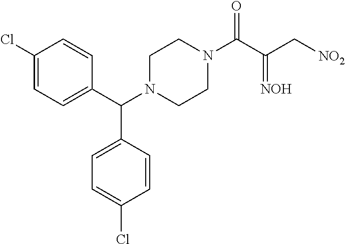 | Ferroptosis inducer by GPX4 inhibition |
| JKE-1716 | C₂₀H₂₀C₁₂N₄O₄ Cas: 2421118-05-8 | 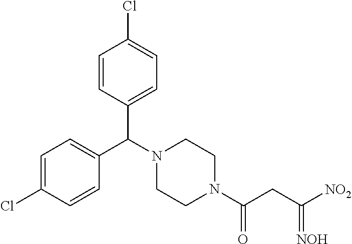 | Ferroptosis inducer by GPX4 inhibition |
| L-buthionine sulfoximine (L- BSO) | C₈H₁₈N₂O₃S Cas: 83730-53-4 | 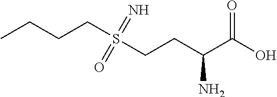 | Ferroptosis inducer by GCL and GSS inhibition |
| ML-162 | C₂₃H₂₂C₁₂N₂O₃S Cas: 1035072-16-2 | 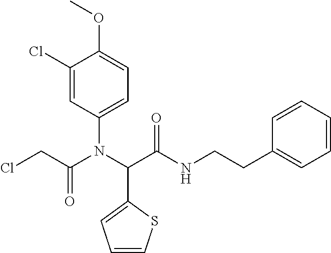 [SMILES: COc1ccc(N(C(=O)CCl)C(C(=O)NCCc2ccccc2)c2cccs2)cc1Cl] | Ferroptosis inducer by GPX4 inhibition |
| ML-210 | C₂₂H₂₀Cl₂N₄O₄ Cas: 1360705-96-9 | 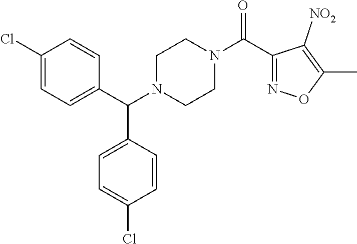 [SMILES: Cc1onc(C(=O)N2CCN(C(c3ccc(Cl)cc3)c3ccc(Cl)cc3)CC2)c1[N+](=O)[O-]] | Ferroptosis inducer by GPX4 inhibition |
| RSL3-like (Formula XIV) | | Formula XIV: 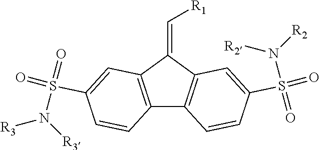 | Ferroptosis inducer by GPX4 inhibition |
| | | R₁ is selected from the group consisting of H, OH, and | |
| | | —(OCH₂CH₂)ₓOH; X is an integer from 1 to 6; and R₂, | |
| | | R₂′, R₃, independently are selected from the group consisting | |
| | | of H, C₃₋₈cycloalkyl, and | |
| | | combinations thereof, or R₂ and R₂′ may be joined | |
| | | together to form a pyridinyl or pyranyl and R₃ and R₃′ | |
| | | may be joined together to form a pyridinyl or pyranyl. | |
| RSL3-like (Formula XV) | | Formula XV: 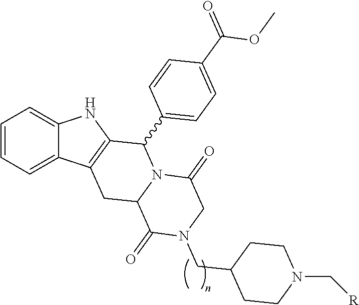 | Ferroptosis inducer by GPX4 inhibition |
| | | or an N-oxide, crystalline form, hydrate, or | |
| | | pharmaceutically acceptable salt thereof; wherein: n is | |
| | | 2, 3 or 4; and R is a substituted or unsubstituted | |
| | | C₁-_{C6}alkyl group, a substituted or unsubstituted | |
| | | C₃-C₁₀cycloalkyl group, a substituted or unsubstituted | |
| | | C₂-C₈heterocycloalkyl group, a substituted or unsubstituted | |
| | | C₆-C₁₀ aromatic ring group, or a substituted or | |
| | | unsubstituted C₃-C₆heteroaryl ring group, wherein the | |
| | | substitution means that one or more hydrogen atoms in | |
| | | each group are substituted by the following groups | |
| | | selected from the group consisting of: halogen, cyano, | |
| | | nitro, hydroxy, C₁-C₆alkyl, halogenated C₁-C₆alkyl, | |
| | | C₁-C₆alkoxy, halogenated C₁-C₆alkoxy, COOH | |
| | | (carboxy), COOC₁-C₆alkyl, OCOC₁-C₆alkyl. | |
| Se- Methyl- selenocysteine (hydrochloride) | C4H₉NO₂Se•HCl Cas: 863394-07-4 | 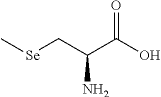 | Ferroptosis inducer by preventing increases in glutathione reductase |
| | | •HCl | and glutantione |
| | | | peroxidase (GPX) |
| | | | activity |
| simvastatin | C₂₅H₃₈O₅ Cas: 79902-63-9 | 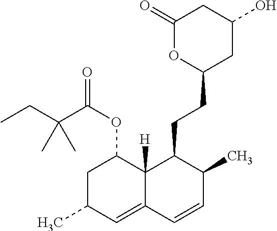 [SMILES: CCC(C)(C)C(=O)O[C@H]1C[C@@H](C)C=C2C=C[C@H](C)[C@H](CC[C@@H]3C[C@@H](O)CC(=O)O3)[C@H]21] | Ferroptosis inducer by GPX4 inhibition |
| sorafenib | C₂₁H₁₆ClF₃N₄O₃ Cas: 284461-73-0 | 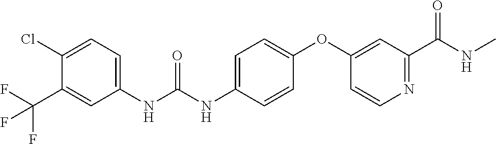 [SMILES: CNC(=O)c1cc(Oc2ccc(NC(=O)Nc3ccc(Cl)c(C(F)(F)F)c3)cc2)ccn1] | Induces ferroptosis by inhibiting multiple kinases |
| sulfasalazine | C₁₈H₁₄N₄O₅S Cas: 599-79-1 | 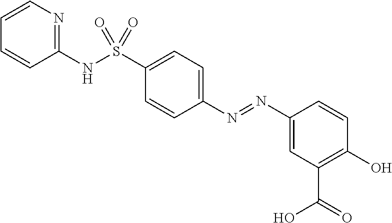 [SMILES: O=C(O)c1cc(/N=N/c2ccc(S(=O)(=O)Nc3ccccn3)cc2)ccc1O] | Ferroptosis inducer by xCT inhibition |
| trigonelline | C₇H₈NO₂•Cl Cas: 6138-41-6 | 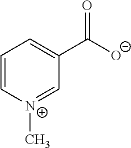 | Ferroptosis inducer by NRF2 inhibition. NRF2 is a transcriptional regulator of GPX4 protein content |

In some embodiments, the ferroptosis-modulating agent is selected from the group consisting of: (1S,3R)-RSL3, ML-162, ML-210, JKE-1674, JKE-1716, erastin, jacaric acid, buthionine sulfoximine (BSO), trigonelline, glutamate, sulfasalazine, auranofin, brusatol, sorafenib, sorafenib-d3, sorafenib tosylate, trigonelline, FIN56, FINO2, CIL56, dihydroisotanshinone I, GPX4-IN-3, analogs, salts, or derivatives thereof. In some embodiments, the agent in Table 1 is a pharmaceutically acceptable salt form of the small molecule.

### (2) Priming Agents

Provided herein are methods of inducing targeted cell death in a mammalian tissue in vivo, the methods comprising: (a) contacting a mammalian tissue with a priming agent; (b) contacting the mammalian tissue in vivo with an effective amount of a ferroptosis-inducing agent for a duration of time of at least 4 hours, when a plurality of cells within the mammalian tissue are responsive to the priming agent as determined by detecting in the mammalian tissue: (i) a plurality of cells comprising a concentration of selenium greater than a selenium concentration in the mammalian tissue prior to contacting with the priming agent; (ii) a plurality of cells comprising a concentration of iron greater than an iron concentration in the mammalian tissue prior to contacting with the priming agent; (iii) a plurality of cells comprising a PUFA concentration greater than a PUFA concentration in the mammalian tissue prior to contacting with the priming agent; (iv) a plurality of cells expressing one or more markers indicative of a mesenchymal state; (v) a plurality of cells comprising a peroxidizability index (PI) greater than a PI in the mammalian tissue prior to contacting with the priming agent; and/or (vi) hyperproliferation of cells in the mammalian tissue, wherein the ferroptosis-inducing agent induces targeted cell death in the mammalian tissue in vivo. In some embodiments, a priming agent is administered prior to the administration of a ferroptosis-inducing agent provided herein. In some embodiments, the priming agent is administered in vivo, in vitro, or ex vivo. A priming agent is an agent that prepares a subject or tissue for administration of a therapeutically effective dose of a ferroptosis-inducing or ferroptosis-modulating agent provided herein. In some embodiments, the priming agent is a ferroptosis-inhibitor. In some embodiments, the priming agent renders a cell within a tissue as ferroptosis-sensitive. In some embodiments, the priming agent is a lipophilic antioxidant or radical trapping agent. In some embodiments, the priming agent is a polyunsaturated fatty acid. In some embodiments, the priming agent is an iron chelator. In some embodiments, the priming agent is a lipid peroxidation inhibitor. In some embodiments, the priming agent modulates blood oxygen levels. In some embodiments the priming agent is a hydroperoxide. In some embodiments, the priming agent is selected from the group consisting of: liproxstatin-1, ferrostatin-1, deferoxamine (DFO), iron, selenium, vitamin E, erythropoietin, a polyunsaturated fatty acid, N-acetylcysteine, pifithrin-alpha-HBr, and methylnaphthalene-4-propionate endoperoxide (MNPE). In some embodiments, the polyunsaturated fatty acid is selected from the group consisting of: hexadecatrienoic acid (HTA), alpha-linolenic acid (ALA), stearidonic acid (SDA), eicosatrienoic acid (ETE), eicosatetraenoic acid (ETA), eicosapentaenoic acid (EPA, Timnodonic acid), heneicosapentaenoic acid (HPA), docosapentaenoic acid (DPA, Clupanodonic acid), docosahexaenoic acid (DHA, Cervonic acid), tetracosahexaenoic acid (Nisinic acid), tetracosapentaenoic acid, linoleic acid (LA), gamma-linolenic acid (GLA), eicosadienoic acid, dihomo-gamma-linolenic acid (DGLA), arachidonic acid (AA), docosadienoic acid, adrenic acid (AdA), docosapentaenoic acid (Osbond acid), tetracosatetraenoic acid, and tetracosapentaenoic acid. Non-limiting examples of priming agents are provided in Table 2.

TABLE 2
Priming Agents.

| | Chemical | | Target/Mechanism |
|---|---|---|---|
| Name | Formula/CAS | Chemical Structure | of Action |
| liproxstatin-1 | C₁₉H₂₁ClN₄ Cas: 950455- 15-9 | 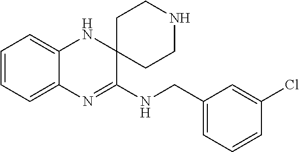 [SMILES: Clc1cccc(CNC2=Nc3ccccc3NC23CCNCC3)c1] | Modulates ferroptosis by inhibiting GCL and GSS |
| ferrostatin-1 | C₁₅H₂₂N₂O₂ Cas: 347174- 05-4 | 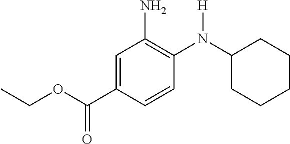 [SMILES: CCOC(=O)c1ccc(NC2CCCCC2)c(N)c1] | Modulates ferroptosis by inhibiting lipid peroxidation |
| deferoxamine mesylate (DFO) | C₂₆H₅₂N₆O₁₁S Cas: 138-14-7 | 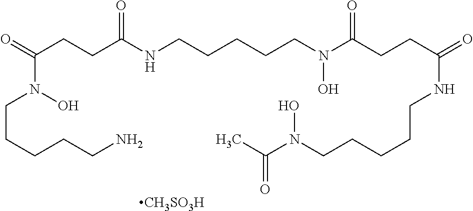 | Modulates ferroptosis by chelating iron |
| iron | Fe | Fe⁺² | Modulates |
| | | | ferroptosis iron- |
| | | | dependent cell death |
| | | | by multiple |
| | | | mechanisms |
| selenium | Se | Se | Modulates |
| | | | ferroptosis by |
| | | | increasing cellular |
| | | | glutathione |
| | | | peroxidase activity |
| | | | and reduces |
| | | | susceptibility |
| | | | to lipid |
| | | | peroxidation |
| Se- Methylselenocysteine (hydrochloride0 | C₄H₉NO₂Se•HCl Cas: 863394-07-4 | 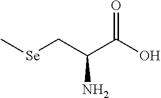 •HCl | Modulates ferroptosis by preventing increases in glutathione reductase and glutathione peroxidase (GPX) activity, as well as decreases in glutathione (GSH) levels |
| vitamin E (α- tocopherol) | C₃₁H₅₂O₃ Cas: 7695-91- 2 | 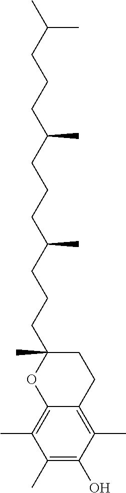 | Modulates ferroptosis by lipoxygenase inhibition |
| α-tocotrienol | C₂₉H₄₄O₂ Cas: 58864- 81-6 | 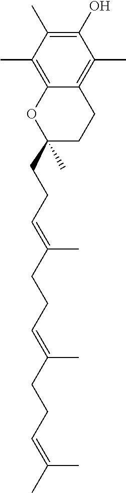 | Modulates ferroptosis by lipoxygenase inhibition |
| Trolox | C₁₄H₁₈O₄ Cas: 53188-07-1 | 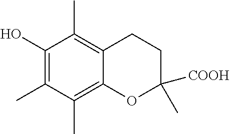 [SMILES: Cc1c(C)c2c(c(C)c1O)CCC(C)(C(=O)O)O2] | Derivative of vitamin E with potent antioxidant properties. Modulates ferroptosis by lipoxygenase inhibition |
| erythropoietin | | erythropoietin precursor [Homo sapiens] | glycoprotein |
| polypeptide | | NCBI Reference Sequence: NP_000790.2 | cytokine that |
| | | MGVHECPAWLWLLSLLSLPLGLPVLGAPPR | modulates |
| | | LICDSRVLERYLLEAKEAENITTGCAEHCSL | ferroptosis by |
| | | NENITVPDTKVNFYAWKRMEVGQQAVEVWQG | increasing systemic |
| | | LALLSEAVLRGQALLVNSSQPWEPLQLHVDK | oxygen capacity by |
| | | AVSGLRSLTTLLRALGAQKEAISPPDAASAA | stimulating the |
| | | PLRTITADTFRKLFRVYSNFLRGKLKLYTGE | activity of GPX4 |
| | | ACRTGDR | and inhibiting lipid |
| | | erythropoietin [Homo sapiens] | peroxides |
| | | NCBI Reference Sequence: GenBank: | |
| | | AGW15567.1 | |
| | | MGVHECPAWLWLLSLLSLPLGLPVLGAPPR | |
| | | LICDSRVLERYLLEAKEAENITTGCAEHCSL | |
| | | NENITVPDTKVNFYAWKRMEVGQQAVEVWQG | |
| | | LALLSEAVLRGQALLVNSSQPWEPLQLHVDK | |
| | | AVSGLRSLTTLLRALGAQKEAISPPDAASAA | |
| | | PLRTITADTFRKLFRVYSNFLRGKLKLYTGE | |
| | | ACRTGDRV | |
| N-Acetylcysteine amide | C₅H₁₀N₂O₂S Cas: 38520- 57-9 | 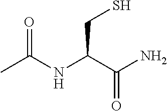 | Modulates ferroptosis by inhibiting glutamate- induced cytotoxicity, decreases in intracellular glutathione (GSH) levels, and increases in intracellular reactive oxygen species (ROS) levels |
| pifithrin-alpha-HBr | C₁₆H₁₈N₂OS•xHBr Cas: 63208- 82-2 | 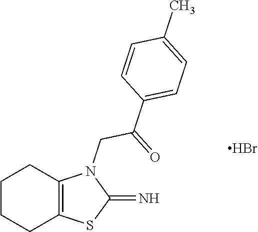 [SMILES: Cc1ccc(C(=O)Cn2c3c(sc2=N)CCCC3)cc1 ; Br] | Modulates cell death and ferroptosis by p53 inhibition |
| RC574 | C₁₈H₂₄OSSe Cas: 2584111- 87-8 | 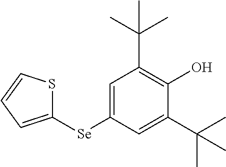 | Increases glutathione peroxidase 1 (GPX1) levels and GPX activity, inhibits ferroptosis induced by GPX4 inhibition |

In some embodiments, methods provided herein comprise administering any one of the agents listed in Table 2. Further provided herein are pharmaceutical compositions, wherein the compositions comprise a ferroptosis-inducing agent or ferroptosis-modulating agent and a priming agent. In some embodiments, the pharmaceutical compositions further comprise a chemotherapeutic agent.

In some embodiments are compositions for use in methods of reducing adipose cell number in a subject in need thereof. In some embodiments are compositions for use in the regulation, modulation, or induction of weight loss, or fat loss. In some embodiments are compositions for use in the regulation or modulation, of iron metabolism. In some embodiments are compositions for use in the treatment of obesity. In some embodiments are compositions for use in the treatment of a kidney disease. In some embodiments are compositions for use in the treatment of a kidney disease. In some embodiments are compositions for use in the treatment of a disease or condition related to iron metabolism.

In some embodiments are compositions comprising an agent in Table 1 used in the manufacture of a medicament for the treatment of a disease or condition described herein such as a kidney disease or kidney condition. In some embodiments are compositions comprising an agent in Table 1 used in the manufacture of a medicament for the treatment of elevated cholesterol. In some embodiments are compositions comprising an agent in Table 1 used in the manufacture of a medicament for inducing ferroptosis in a subject. In some embodiments are compositions comprising a ferroptosis inducing agent used in the manufacture of a medicament for the treatment of a kidney disease, a kidney condition, a disease or condition related to fat metabolism, or a disease or condition related to elevated cholesterol.

### (3) Additional Treatments

In some embodiments, the methods provided herein comprise administering at least one additional treatment (e.g., a second weight loss therapy) to a subject. In some cases, the second weight therapy is selected from: a GLP-1 agonist, a gastric inhibitory polypeptide analog, a naltrexone-bupropion or a salt thereof, an orlistat or a salt thereof, a phentermine or a salt thereof, and a phentermine-topiramate or a salt thereof, a selenium, an exercise therapy, a surgery, a diet modification, and any combination thereof. In some cases, the GLP-1 agonist comprises a dulaglutide, an exenatide, a semaglutide, a liraglutide, a lixisenatide, or a salt of any of these. In some cases, the gastric inhibitory polypeptide comprises tirzepatide or a derivative thereof. In some cases, the surgery is a gastric sleeve, a gastric bypass, an adjustable gastric band, or any combination thereof. In some cases, a selenium can comprise inorganic selenium (selenate and selenite) organic selenium (selenomethionine and selenocysteine) or any combination thereof.

In some embodiments, the methods provided herein comprise administering at least one additional treatment to a subject. In some embodiments, the additional treatment is surgery. In some embodiments, the additional treatment is radiation therapy. In some embodiments, the additional treatment is a dietary supplement. Non-limiting examples of dietary supplements include: probiotics, selenium, iron, vitamins (e.g., vitamin A, vitamin C, vitamin E), curcumin, fish oils, beta carotene, hydrogen sulfides, fatty acids, methionine, cysteine, homocysteine, taurine, cystine or di-cysteine. In some embodiments, the dietary supplement is a high-selenium nutritional supplement.

In some embodiments, the additional treatment is an additional therapeutic agent. In some embodiments, the methods provided herein comprise administering an additional agent in combination with a ferroptosis-inducing agent, an iron-dependent cell death inducing agent, and/or a priming agent provided herein. In some embodiments, the additional agent is a cell-death inducing agent. In some embodiments, the additional agent is an anti-cancer agent. In some embodiments, the anti-cancer agent is a chemotherapeutic agent. A chemotherapeutic agent or compound is any agent or compound useful in the treatment of cancer. The chemotherapeutic cancer agents that can be used in combination with ferroptosis-inducing agents or iron-dependent cell death agents provided herein which include, but are not limited to, mitotic inhibitors (vinca alkaloids). These include vincristine, vinblastine, vindesine and Navelbine™ (vinorelbine, 5′-noranhydroblastine). In yet other cases, chemotherapeutic cancer agents include topoisomerase I inhibitors, such as camptothecin compounds. As used herein, “camptothecin compounds” include Camptosar™ (irinotecan HCL), Hycamtin™ (topotecan HCL) and other compounds derived from camptothecin and its analogues. Another category of chemotherapeutic cancer agents that can be used in the methods and compositions disclosed herein are podophyllotoxin derivatives, such as etoposide, teniposide and mitopodozide. The present disclosure further encompasses other chemotherapeutic cancer agents known as alkylating agents, which alkylate the genetic material in tumor cells. These include without limitation cisplatin, cyclophosphamide, nitrogen mustard, trimethylene thiophosphoramide, carmustine, busulfan, chlorambucil, belustine, uracil mustard, chlomaphazin, and dacarbazine. The disclosure encompasses antimetabolites as chemotherapeutic agents. Examples of these types of agents include cytosine arabinoside, fluorouracil, methotrexate, mercaptopurine, azathioprime, and procarbazine. An additional category of chemotherapeutic cancer agents that may be used in the methods and compositions disclosed herein include antibiotics. Examples include without limitation doxorubicin, bleomycin, dactinomycin, daunorubicin, mithramycin, mitomycin, mytomycin C, and daunomycin. There are numerous liposomal formulations commercially available for these compounds. The present disclosure further encompasses other chemotherapeutic cancer agents including without limitation anti-tumor antibodies, dacarbazine, azacytidine, amsacrine, melphalan, ifosfamide and mitoxantrone.

The disclosed agents provided herein can be administered in combination with other anti-tumor agents, including cytotoxic/antineoplastic agents and anti-angiogenic agents. Cytotoxic/anti-neoplastic agents can be defined as agents who attack and kill cancer cells. Some cytotoxic/anti-neoplastic agents can be alkylating agents, which alkylate the genetic material in tumor cells, e.g., cis-platin, cyclophosphamide, nitrogen mustard, trimethylene thiophosphoramide, carmustine, busulfan, chlorambucil, belustine, uracil mustard, chlomaphazin, and dacabazine. Other cytotoxic/anti-neoplastic agents can be antimetabolites for tumor cells, e.g., cytosine arabinoside, fluorouracil, methotrexate, mercaptopuirine, azathioprime, and procarbazine. Other cytotoxic/anti-neoplastic agents can be antibiotics, e.g., doxorubicin, bleomycin, dactinomycin, daunorubicin, mithramycin, mitomycin, mytomycin C, and daunomycin. There are numerous liposomal formulations commercially available for these compounds. Still other cytotoxic/anti-neoplastic agents can be mitotic inhibitors (vinca alkaloids). These include vincristine, vinblastine and etoposide. Miscellaneous cytotoxic/anti-neoplastic agents include taxol and its derivatives, L-asparaginase, anti-tumor antibodies, dacarbazine, azacytidine, amsacrine, melphalan, VM-26, ifosfamide, mitoxantrone, and vindesine.

Anti-angiogenic agents can also be used. Suitable anti-angiogenic agents for use in the disclosed methods and compositions include anti-VEGF antibodies, including humanized and chimeric antibodies, anti-VEGF aptamers and antisense oligonucleotides. Other inhibitors of angiogenesis include angiostatin, endostatin, interferons, interleukin 1 (including α and β) interleukin 12, retinoic acid, and tissue inhibitors of metalloproteinase-1 and -2. (TIMP-1 and -2). Small molecules, including topoisomerases such as razoxane, a topoisomerase II inhibitor with anti-angiogenic activity, can also be used.

Other anti-cancer agents that can be used in combination with the ferroptosis-inducing agents provided herein can include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; avastin; azacitidine; azetepa; azotomycin; batimastat; benzodepa; bevacizumab; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; folinic acid; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan, piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer agents include, but are not limited to: 20-epi-1,25 dihydroxyvitamin D3; 5-ethynyluracil; abiraterone; aclarubicin; acylfulvene; adecypenol; adozelesin; aldesleukin; ALL-TK agonist or antagonist; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists or agonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin, prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A, sargramostim; Sdi 1 mimetics; semustine; senescence derived inhibitor 1; sense oligonucleotides; signal transduction inhibitors; signal transduction modulators; single chain antigen binding protein, sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor agonists or antagonists; vapreotide; variolin B; erythrocyte gene therapy; velaresol; veramine; verdins; verteporfin; vinorelbine; vinxaltine; vitaxin; vorozole; zanoterone; zeniplatin; zilascorb; and zinostatin stimalamer. Any of the aforementioned chemotherapeutics can be administered at a clinically effective dose. A chemotherapeutic can also be administered from about day: −14, −13, −12, −11, −10, −9, −8, −7, −6, −5, −4, −3, −2, −1, 0, 1, 2, 3, 4, 5, 6, 7, 8, 9, 10, 11, 12, 13, or up to about day 14 after administration of an agent provided herein. In some cases, a subject can have a refractory cancer that is unresponsive to a chemotherapeutic.

### Pharmaceutical Compositions

Provided herein are pharmaceutical compositions, wherein the pharmaceutical compositions comprise an agent selected from Table 1 or a combination of agents selected from Table 1 and/or Table 2; and a pharmaceutically acceptable excipient. In some embodiments, the pharmaceutical composition further comprises a cell death inducing agent. In some embodiments, the pharmaceutical composition further comprises a chemotherapeutic agent. In some embodiments, pharmaceutical compositions provided herein are in a suspension, optionally a homogeneous suspension. In some embodiments, pharmaceutical compositions provided herein are in an emulsion form. In some embodiments, pharmaceutical compositions provided herein comprise a salt form of any one of the agents provided herein. In some embodiments, the salt is a methanesulfonate salt.

Also provided herein is a pharmaceutical composition comprising a ferroptosis-inducing agent or an iron-dependent cell death agent provided herein. Also provided herein is a pharmaceutical composition comprising a ferroptosis-modulating agent. In some cases, the pharmaceutical composition can be in unit dose form. In some embodiments, agents provided herein are combined with pharmaceutically acceptable salts, excipients, and/or carriers to form a pharmaceutical composition. Pharmaceutical salts, excipients, and carriers may be chosen based on the route of administration, the location of the target issue, and the time course of delivery of the drug. A pharmaceutically acceptable carrier or excipient may include solvents, dispersion media, coatings, antibacterial and antifungal agents, isotonic and absorption delaying agents, etc., compatible with pharmaceutical administration.

In some embodiments, the pharmaceutical composition is in the form of a solid, semi-solid, liquid or gas (aerosol). Injectable preparations, for example, sterile injectable aqueous or oleaginous suspensions may be formulated according to the known art using suitable dispersing or wetting agents and suspending agents. The sterile injectable preparation may also be a sterile injectable solution, suspension, or emulsion in a nontoxic parenterally acceptable diluent or solvent. Among the acceptable vehicles and solvents that may be employed are water, Ringer's solution, U.S.P., and isotonic sodium chloride solution. In addition, sterile, fixed oils are employed as a solvent or suspending medium. For this purpose, any bland fixed oil can be employed including synthetic mono- or diglycerides. In addition, fatty acids such as oleic acid are used in the preparation of injectables. The injectable formulations can be sterilized, for example, by filtration through a bacteria-retaining filter, or by incorporating sterilizing agents in the form of sterile solid compositions which can be dissolved or dispersed in sterile water or other sterile injectable medium prior to use.

Exemplary carriers and excipients can include dextrose, sodium chloride, sucrose, lactose, cellulose, xylitol, sorbitol, malitol, gelatin, polymers, polyethylene glycol (PEG), polyvinylpyrrolidone (PVP), and any combination thereof. In some embodiments, an excipient such as dextrose or sodium chloride can be at a percent from about 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, or up to about 15%.

Solid dosage forms for oral administration include capsules, tablets, pills, powders, and granules. In such solid dosage forms, the encapsulated or unencapsulated conjugate is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar-agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may also comprise buffering agents.

Tablets may be either film coated or enteric coated according to methods known in the art. Liquid preparations for oral administration can take the form of, for example, solutions, syrups, or suspensions, or they can be presented as a dry product for reconstitution with water or other suitable vehicle before use. Such liquid preparations can be prepared by conventional means with pharmaceutically acceptable carriers and additives, for example, suspending agents, e.g., sorbitol syrup, cellulose derivatives, or hydrogenated edible fats; emulsifying agents, for example, lecithin or acacia; non-aqueous vehicles, for example, almond oil, oily esters, ethyl alcohol, or fractionated vegetable oils; and preservatives, for example, methyl or propyl-p-hydroxybenzoates or sorbic acid. The preparations can also contain buffer salts, flavoring, coloring, and/or sweetening agents as appropriate. If desired, preparations for oral administration can be suitably formulated to give controlled release of the active compound.

Formulations suitable for buccal (sublingual) administration include, for example, lozenges containing the active compound in a flavored base, usually sucrose and acacia or tragacanth; and pastilles containing the compound in an inert base such as gelatin and glycerin or sucrose and acacia.

Ferroptosis-inducing agents or ferroptosis-modulating agents provided herein can be formulated as a rectal composition, for example, suppositories or retention enemas, for example, containing conventional suppository bases, for example, cocoa butter or other glycerides, or gel forming agents, such as carbomers.

Pharmaceutical compositions also can be administered by controlled release formulations and/or delivery devices (see, e.g., in U.S. Pat. No. 5,733,566).

Various delivery vehicles are known and can be used to administer ferroptosis-inducing agents or ferroptosis-modulating agents provided herein, such as but not limited to, encapsulation in liposomes, microparticles, microcapsules, nanoparticles, vectors, and recombinant cells. Liposomes and/or nanoparticles also can be employed with administration of compositions herein. Liposomes are formed from phospholipids that are dispersed in an aqueous medium and spontaneously form multilamellar concentric bilayer vesicles (also termed multilamellar vesicles (MLVs)). MLVs generally have diameters of from 25 nm to 4 μm. Sonication of MLVs results in the formation of small unilamellar vesicles (SUVs) with diameters in the range of 200 to 500 angstroms containing an aqueous solution in the core.

Phospholipids can form a variety of structures other than liposomes when dispersed in water, depending on the molar ratio of lipid to water. At low ratios, the liposomes form. Physical characteristics of liposomes depend on pH, ionic strength and the presence of divalent cations. Liposomes can show low permeability to ionic and polar substances, but at elevated temperatures undergo a phase transition which markedly alters their permeability. The phase transition involves a change from a closely packed, ordered structure, known as the gel state, to a loosely packed, less-ordered structure, known as the fluid state. This occurs at a characteristic phase-transition temperature and results in an increase in permeability to ions, sugars and drugs.

Liposomes interact with cells via different mechanisms: endocytosis by phagocytic cells of the reticuloendothelial system such as macrophages and neutrophils; adsorption to the cell surface, either by nonspecific weak hydrophobic or electrostatic forces, or by specific interactions with cell-surface components; fusion with the plasma cell membrane by insertion of the lipid bilayer of the liposome into the plasma membrane, with simultaneous release of liposomal contents into the cytoplasm; and by transfer of liposomal lipids to cellular or subcellular membranes, or vice versa, without any association of the liposome contents. Varying the liposome formulation can alter which mechanism is operative, although more than one can operate at the same time. Nanocapsules can generally entrap compounds in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles can also be used as a delivery vehicle.

Nanoparticle carriers that specifically target a tissue provided herein may also be used as a pharmaceutically acceptable carrier. In some embodiments, the nanoparticle is a gold nanoparticle, a platinum nanoparticle, an iron-oxide nanoparticle, a lipid nanoparticle, a selenium nanoparticle, a tumor-targeting glycol chitosan nanoparticle (CNP), a cathepsin B sensitive nanoparticle, a hyaluronic acid nanoparticle, a paramagnetic nanoparticle, or a polymeric nanoparticle.

Suitable pharmaceutical formulations of ferroptosis-inducing agents or ferroptosis-modulating agents for transdermal application include an effective amount of an agent with a carrier. Carriers include absorbable pharmacologically acceptable solvents to assist passage through the skin of the subject. For example, transdermal devices are in the form of a bandage or patch comprising a backing member, a reservoir containing the compound optionally with carriers, optionally a rate controlling barrier to deliver the compound to the skin of the host at a controlled and predetermined rate over a prolonged period of time, and a means to secure the device to the skin. Matrix transdermal formulations may also be used. Suitable formulations for topical application, e.g., to the skin and eyes, are preferably aqueous solutions, ointments, creams or gels well-known in the art. The formulations may contain solubilizers, stabilizers, tonicity enhancing agents, buffers and preservatives.

In certain embodiments, ferroptosis-inducing agents or ferroptosis-modulating agents provided herein are formulated as a depot composition. Such long-acting formulations can be administered by implantation (for example, subcutaneously or intramuscularly) or by intramuscular injection. The ferroptosis-inducing agents or ferroptosis-modulating agents can be formulated with suitable polymeric or hydrophobic materials (for example as an emulsion in an acceptable oil), ion exchange resins, biodegradable polymers, or as sparingly soluble derivatives, for example, as a sparingly soluble salt.

In some embodiments, one or more agent provided herein is formulated as a pharmaceutical food composition (also referred to as a medical food). The food composition can be for consumption by a mammal, for example by a human or a non-human mammal. Agents provided herein can be formulated as a dietary supplement or a medical food. In some embodiments, agents provided herein are administered with a food ingredient. A food ingredient is any product, composition, or a component of a food known to have or disclosed as having a nutritional effect. Food can include various meats (e.g., beef, pork, poultry, fish, etc.), dairy products (e.g., milk, cheese, eggs), fruits, vegetables, cereals, breads, etc., and components thereof. Food can be fresh or preserved, e.g., by canning, dehydration, freezing, or smoking. Food can be provided in raw, unprepared and/or natural states or in cooked, prepared, and/or combined states. In some embodiments, the food ingredient is selected from the group consisting of: fat, carbohydrates, protein, fiber, nutritional balancing agent, and mixtures thereof. In some embodiments, the pharmaceutical food composition provided herein further comprises one or more of a protein or an amino acid. In some embodiments of any of the aspects, the pharmaceutical food composition further comprises adenine, one or more vitamins (e.g., vitamin E), potassium, fatty acids, and/or calcium carbonate.

### Methods of Administering an Agent

Provided herein can be methods for administering a therapeutic regime to a subject having a disease or disorder (e.g., cancer, an autoimmune disease, obesity, or fibrosis). In some embodiments, the administering is sustained administration of a therapeutically effective amount of a ferroptosis-inducing agent or a ferroptosis-modulating agent. In some embodiments, the sustained administration of the ferroptosis-inducing agent comprises providing to a tissue the ferroptosis-inducing agent in an amount sufficient to achieve a distribution of at least about 10 ng/mm² within said tissue for a period of at least 4 hours, thereby inducing ferroptosis in the tissue. In some embodiments, the sustained administration further forms a gradient of a sub-therapeutic amount of the ferroptosis-inducing agent adjacent to an administration site within the tissue. In some embodiments, sustained administration of the ferroptosis-inducing agent or a ferroptosis-modulating agent comprises additional administration steps. In some embodiments, the ferroptosis-inducing agent or the ferroptosis-modulating agent is administered more than once. In some embodiments, the administering is via a system provided herein. In some embodiments, the administering local administration within a tissue. In some embodiments, the tissue is contacted in vivo with an effective amount of an iron-dependent cell death agent for a duration of time of at least 4 hours. In some embodiments, the administering comprises contacting a mammalian tissue with a priming agent and contacting the mammalian tissue with an effective amount of a ferroptosis-inducing agent provided herein, wherein the ferroptosis-inducing agent induces targeted cell death in the mammalian tissue in vivo. In some embodiments, the administering is local administration or systemic administration. In some embodiments, the administering or contacting step is via intratumoral injection, oral administration, transdermal injection, inhalation, nasal administration, topical administration, vaginal administration, ophthalmic administration, intracerebral administration, rectal administration.

In some instances, an agent or combination of agents provided herein are administered as a unit dosage form. Many agents can be administered orally as liquids, capsules, tablets, or chewable tablets. Because the oral route is the most convenient and usually the safest and least expensive, it is the one most often used. However, it has limitations because of the way a drug typically moves through the digestive tract. For agents administered orally, absorption may begin in the mouth and stomach. However, most agents are usually absorbed from the small intestine. The drug passes through the intestinal wall and travels to the liver before being transported via the bloodstream to its target site. The intestinal wall and liver chemically alter (metabolize) many agents, decreasing the amount of drug reaching the bloodstream. Consequently, these agents are often given in smaller doses when injected intravenously to produce the same effect.

In some embodiments, an agent provided herein is formulated for oral administration. In some embodiments, an agent provided herein is formulated for administration/for use in administration via a subcutaneous, intradermal, intramuscular, inhalation, intravenous, intraperitoneal, intracranial, intrathecal, intratumoral, or oral route. For a subcutaneous route, a needle is inserted into fatty tissue just beneath the skin. After a drug is injected, it then moves into small blood vessels (capillaries) and is carried away by the bloodstream. Alternatively, a drug reaches the bloodstream through the lymphatic vessels. The intramuscular route is preferred to the subcutaneous route when larger volumes of a drug product are needed. Because the muscles lie below the skin and fatty tissues, a longer needle is used. Agents are usually injected into the muscle of the upper arm, thigh, or buttock. How quickly the drug is absorbed into the bloodstream depends, in part, on the blood supply to the muscle: The sparser the blood supply, the longer it takes for the drug to be absorbed. For the intravenous route, a needle is inserted directly into a vein. A solution containing the drug may be given in a single dose or by continuous infusion. For infusion, the solution is moved by gravity (from a collapsible plastic bag) or, more commonly, by an infusion pump through thin flexible tubing to a tube (catheter) inserted in a vein, usually in the forearm. In some cases, agents or therapeutic regimes are administered as infusions. An infusion can take place over a period of time. For example, an infusion can be an administration of an agent or therapeutic regime over a period of about 5 minutes to about 5 hours. An infusion can take place over a period of about 5 min, 10 min, 20 min, 30 min, 40 min, 50 min, 1 hour, 1.5 hours, 2 hours, 2.5 hours, 3 hours, 3.5 hours, 4 hours, 4.5 hours, or up to about 5 hours.

In some embodiments, intravenous administration is used to deliver a precise dose quickly and in a well-controlled manner throughout the body. It is also used for irritating solutions, which would cause pain and damage tissues if given by subcutaneous or intramuscular injection. An intravenous injection can be more difficult to administer than a subcutaneous or intramuscular injection because inserting a needle or catheter into a vein may be difficult, especially if the person is obese. When given intravenously, a drug is delivered immediately to the bloodstream and tends to take effect more quickly than when given by any other route. Consequently, health care practitioners closely monitor people who receive an intravenous injection for signs that the drug is working or is causing undesired side effects. Also, the effect of a drug given by this route tends to last for a shorter time. Therefore, some agents must be given by continuous infusion to keep their effect constant. For the intrathecal route, a needle is inserted between two vertebrae in the lower spine and into the space around the spinal cord. The drug is then injected into the spinal canal. A small amount of local anesthetic is often used to numb the injection site. This route is used when a drug is needed to produce rapid or local effects on the brain, spinal cord, or the layers of tissue covering them (meninges)—for example, to treat infections of these structures.

For administration by inhalation, the ferroptosis-inducing agent or the ferroptosis-modulating agent provided herein can be conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the compound and a suitable powder base, for example, lactose or starch. Agents administered by inhalation through the mouth can be atomized into smaller droplets than those administered by the nasal route, so that the agents can pass through the windpipe (trachea) and into the lungs. How deeply into the lungs the agents go depends on the size of the droplets. Smaller droplets go deeper, which increases the amount of drug absorbed. Inside the lungs, they are absorbed into the bloodstream.

Agents applied to the skin are usually used for their local effects and thus are most commonly used to treat superficial skin disorders, such as psoriasis, eczema, skin infections (viral, bacterial, and fungal), itching, and dry skin. The drug is mixed with inactive substances. Depending on the consistency of the inactive substances, the formulation may be an ointment, cream, lotion, solution, powder, or gel.

In some cases, a treatment regime may be dosed according to a body weight of a subject. In subjects who are determined obese (BMI>35) a practical weight may need to be utilized. BMI is calculated by: BMI=weight (kg)/[height (m)]².

In some cases, a therapeutic regime can be administered along with a carrier or excipient. Ferroptosis-inducing agents or ferroptosis-modulating agents provided herein can be administered with one or more of a second agent, sequentially, or concurrently, either by the same route or by different routes of administration. When administered sequentially, the time between administrations is selected to benefit, among others, the therapeutic efficacy and/or safety of the combination treatment. In certain embodiments, the agents provided herein can be administered first followed by a second agent, or alternatively, the second agent is administered first followed by the agents of the present disclosure (e.g., ferroptosis-inducing/modulating agents of Table 1). By way of example and not limitation, the time between administrations is about 1 hr, about 2 hr, about 4 hr, about 6 hr, about 12 hr, about 16 hr or about 20 hr. In certain embodiments, the time between administrations is about 1, about 2, about 3, about 4, about 5, about 6, or about 7 more days. In some embodiments, the time between administrations is about 1 week, 2 weeks, 3 weeks, or 4 weeks or more. In some embodiments, the time between administrations is about 1 month or 2 months or more.

In some embodiments, ferroptosis-inducing agents provided herein contact the mammalian tissue for at least about 4 hours, at least about 6 hours, at least about 10 hours, at least about 12 hours, at least about 14 hours, at least about 16 hours, at least about 18 hours, at least about 20 hours, at least about 22 hours, at least about 24 hours, at least about 26 hours, at least about 28 hours, at least about 30 hours, at least about 36 hours, at least about 48 hours, up to 72 hours. In some embodiments, ferroptosis-inducing agents provided herein contact the mammalian tissue for about 4 hours. In some embodiments, ferroptosis-inducing agents provided herein contact the mammalian tissue for about 6 hours. In some embodiments, ferroptosis-inducing agents provided herein contact the mammalian tissue for about 10 hours. In some embodiments, ferroptosis-inducing agents provided herein contact the mammalian tissue for about 12 hours. In some embodiments, ferroptosis-inducing agents provided herein contact the mammalian tissue for about 24 hours. In some embodiments, ferroptosis-inducing agents provided herein contact the mammalian tissue for about 48 hours. In some embodiments, ferroptosis-inducing agents provided herein contact the mammalian tissue for about 72 hours.

When administered concurrently, the agent can be administered separately, at the same time as the second agent, by the same or different routes, or administered in a single pharmaceutical composition by the same route. In certain embodiments, the amount and frequency of administration of the second agent can used standard dosages and standard administration frequencies used for the particular compound.

### Dosing and Tissue Distribution

The methods provided herein comprise administering to a subject an agent or pharmaceutical composition provided herein in an amount effective to induce ferroptosis in a tissue in vivo. Agents and pharmaceutical compositions for administering to a subject in need thereof may be formulated in dosage unit form for ease of administration and uniformity of dosage. A dosage unit form is a physically discrete unit of a composition provided herein appropriate for a subject to be treated. It will be understood, however, that the total usage of compositions provided herein will be decided by the attending physician within the scope of sound medical judgment. For any composition provided herein the therapeutically effective dose can be estimated initially either in cell culture assays or in animal models, such as mice, rabbits, dogs, pigs, or non-human primates. The animal model may also be used to achieve a desirable concentration range and route of administration. Such information can then be used to determine useful doses and routes for administration in humans. Therapeutic efficacy and toxicity of compositions provided herein can be determined by standard pharmaceutical procedures in cell cultures or experimental animals, e.g., ED₅₀ (the dose is therapeutically effective in 50% of the population) and LD₅₀ (the dose is lethal to 50% of the population). The dose ratio of toxic to therapeutic effects is the therapeutic index, and it can be expressed as the ratio, LD₅₀/ED₅₀. Pharmaceutical compositions which exhibit large therapeutic indices may be useful in some embodiments. The data obtained from cell culture assays and animal studies may be used in formulating a range of dosage for human use.

A typical human dose of an agent provided herein (e.g., a ferroptosis-inducing/modulating agent) may be from about 10 μg/kg body weight/day to 10,000 mg/kg/day. In some embodiments, the dose of an agent provided herein is from about 0.1 mg/kg to about 1000 mg/kg, from 1 mg/kg to 1000 mg/kg, 1 mg/kg to 800 mg/kg, from about 1 mg/kg to about 700 mg/kg, from about 2 mg/kg to about 500 mg/kg, from about 3 mg/kg to about 400 mg/kg, 4 mg/kg to about 300 mg/kg, or from about 5 mg/kg to about 200 mg/kg. In certain embodiments, the suitable dosages of the agent can be about 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 125 mg/kg, 150 mg/kg, 175 mg/kg, 200 mg/kg, 250 mg/kg, 300 mg/kg, 400 mg/kg, 500 mg/kg, 600 mg/kg, 700 mg/kg, 800 mg/kg, 900 mg/kg, 1000 mg/kg, 2,000 mg/kg, 3,000 mg/kg, 4,000 mg/kg, 5,000 mg/kg, 6,000 mg/kg, 7,000/mg/kg, 8,000 mg/kg, 9,000 mg/kg, up to 9,600 mg/kg. In some embodiments, the dose of an agent provided herein is from about 100 mg/kg/day to about 6,400 mg/kg/day four times per day. In some embodiments, the dose of an agent provided herein is from about 50 mg/kg/day to about 25 mg/kg/day. In some embodiments, the dose of an agent provided herein is from about 400 mg/kg/day to about 800 mg/kg/day. In certain embodiments, the dose of the agent can be administered once per day or divided into subdoses and administered in multiple doses, e.g., twice, three times, or four times per day.

In some embodiments, agents provided herein are administered in an amount of at least about 10 nanograms (ng) or more, about 20 ng or more, about 30 ng or more, about 40 ng or more, about 50 ng or more, about 60 ng or more, about 70 ng or more, about 80 ng or more, about 90 ng or more, up to 100 ng. In some embodiments, the agent is administered in an amount of at least about 1 microgram (μg) or more, about 5 μg or more, about 10 μg or more, about 20 μg or more, about 30 μg or more, about 40 μg or more, about 50 μg or more, about 60 μg or more, about 70 μg or more, about 80 μg or more, about 90 μg or more, up to 100 μg.

In some embodiments, agents provided herein are administered at a concentration of at least about 0.1 micromolar (μM) or more, about 1 μM or more, about 2 μM or more, about 3 μM or more, about 4 μM or more, about 5 μM or more, about 6 μM or more, about 7 μM or more, about 8 μM or more, about 9 μM or more, about 10 μM or more, about 15 μM or more, about 20 μM or more, about 25 μM or more, about 30 μM or more, about 35 μM or more, about 40 μM or more, about 45 μM or more, about 50 μM or more, about 55 μM or more, about 60 μM or more, about 65 μM or more, about 70 μM or more, about 75 μM or more, about 80 μM or more, about 85 μM or more, about 90 μM or more, about 95 μM or more, about 100 μM or more, about 110 μM or more, about 120 μM or more, about 130 μM or more, about 140 μM or more, about 150 μM or more, about 160 μM or more, about 170 μM or more, about 180 μM or more, about 190 μM or more, about 200 μM or more, about 300 μM or more, about 400 μM or more, about 500 μM or more, up to 1 mM. In some embodiments, agents provided herein are administered at a concentration of at least about 0.1 μM up to about 500 μM. In some embodiments, agents provided herein are administered at a concentration of at least about 1 μM up to 500 μM. In some embodiments, agents provided herein are administered at a concentration of at least about 0.1 μM up to 10 μM. In some embodiments, agents provided herein are administered at a concentration of at least about 1 μM up to 10 μM.

In some embodiments, ferroptosis-inducing agents or ferroptosis-modulating agents provided herein are administered intravenously. In some embodiments, ferroptosis-inducing agents or ferroptosis-modulating agents provided herein are administered intravenously at a concentration of at least about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 80 mg/kg, about 90 mg/kg, about 100 mg/kg, about 200 mg/kg, about 300 mg/kg, about 400 mg/kg, about 500 mg/kg, about 600 mg/kg, about 700 mg/kg, about 800 mg/kg, about 900 mg/kg, about 1000 mg/kg, about 1100 mg/kg, about 1200 mg/kg, about 1300 mg/kg, about 1400 mg/kg, about 1500 mg/kg, about 2000 mg/kg, about 2200 mg/kg, about 2400 mg/kg, up to about 2500 mg/kg.

In some embodiments, ferroptosis-inducing agents or ferroptosis-modulating agents provided herein are administered orally. In some embodiments, ferroptosis-inducing agents or ferroptosis-modulating agents provided herein are administered orally at a concentration of at least about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 80 mg/kg, about 90 mg/kg, about 100 mg/kg, about 200 mg/kg, about 300 mg/kg, about 400 mg/kg, about 500 mg/kg, about 600 mg/kg, about 700 mg/kg, about 800 mg/kg, about 900 mg/kg, about 1000 mg/kg, about 1100 mg/kg, about 1200 mg/kg, about 1300 mg/kg, about 1400 mg/kg, about 1500 mg/kg, about 2000 mg/kg, about 2200 mg/kg, about 2400 mg/kg, up to about 2500 mg/kg. In some embodiments, ferroptosis-inducing agents or ferroptosis-modulating agents provided herein are administered orally at a concentration of about 25 mg/kg once per day. In some embodiments, ferroptosis-inducing agents or ferroptosis-modulating agents provided herein are administered orally at a concentration of about 25 mg/kg twice per day. In some embodiments, ferroptosis-inducing agents or ferroptosis-modulating agents provided herein are administered orally at a concentration of about 1300 mg/kg/day. In some embodiments, ferroptosis-inducing agents or ferroptosis-modulating agents provided herein are administered orally at a concentration of about 2400 mg/kg/day.

The methods provided herein can be characterized by or further comprise measuring the distribution of an agent in a target tissue. Distribution of an agent provided herein can be determined by the amount or concentration of the agent within a square millimeter (mm²) or cubic millimeter (mm³) of tissue. For example, for local administration of an agent to a tumor, the tissue may be from about 6 to 7 mm in diameter, 36 to 42 mm², or 216 to 294 mm³. The data obtained from animal studies may be used in formulating a range of drug distribution in a mammalian tissue. Methods of determining tissue distribution of a drug or agent include, for example, mass spectrometry, chromatography, imaging techniques, and immunoassays. The distribution of an agent provided herein can be determined using a system provided herein.

In some embodiments, the tissue is administered a therapeutic amount of a ferroptosis-inducing agent, wherein administration of comprises providing to a tissue the ferroptosis-inducing agent in an amount sufficient to achieve a desired drug distribution. In some embodiments, agents provided herein achieve a distribution within a tissue of at least about 1 ng/mm² or more, about 5 ng/mm² or more, about 10 ng/mm² or more, about 15 ng/mm² or more, about 20 ng/mm² or more, about 25 ng/mm² or more, about 30 ng/mm² or more, about 35 ng/mm²or more, about 40 ng/mm² or more, about 45 ng/mm² or more, about 50 ng/mm² or more, about 55 ng/mm² or more, about 60 ng/mm² or more, about 65 ng/mm² or more, about 70 ng/mm² or more, about 75 ng/mm² or more, about 80 ng/mm² or more, about 85 ng/mm² or more, about 90 ng/mm²or more, about 95 ng/mm² or more, about 100 ng/mm² or more, about 110 ng/mm² or more, about 120 ng/mm² or more, about 130 ng/mm² or more, about 140 ng/mm² or more, about 150 ng/mm² or more, about 160 ng/mm² or more, about 170 ng/mm² or more, about 180 ng/mm² or more, about 190 ng/mm² or more, about 200 ng/mm² or more, about 300 ng/mm² or more, about 400 ng/mm² or more, up to 500 ng/mm². In some embodiments, agents provided herein achieve a distribution within a tissue of at least about 1 ng/mm³ or more, about 5 ng/mm³ or more, about 10 ng/mm³ or more, about 15 ng/mm³ or more, about 20 ng/mm³ or more, about 25 ng/mm³ or more, about 30 ng/mm³ or more, about 35 ng/mm³ or more, about 40 ng/mm³ or more, about 45 ng/mm³ or more, about 50 ng/mm³ or more, about 55 ng/mm³ or more, about 60 ng/mm³ or more, about 65 ng/mm³ or more, about 70 ng/mm³ or more, about 75 ng/mm³ or more, about 80 ng/mm³ or more, about 85 ng/mm³ or more, about 90 ng/mm³ or more, about 95 ng/mm³ or more, about 100 ng/mm³ or more, about 110 ng/mm³ or more, about 120 ng/mm³ or more, about 130 ng/mm³ or more, about 140 ng/mm³ or more, about 150 ng/mm³ or more, about 160 ng/mm³ or more, about 170 ng/mm³ or more, about 180 ng/mm³ or more, about 190 ng/mm³ or more, about 200 ng/mm³ or more, about 300 ng/mm³ or more, about 400 ng/mm³ or more, up to 500 ng/mm³.

In some embodiments, ferroptosis-inducing agents or ferroptosis-modulating agents provided herein are administered at least about once per day, twice per day, three times per day, four times per day, or five times per day. In some embodiments of any of the aspects, ferroptosis-inducing agents or ferroptosis-modulating agents are administered at least about every week, at least about every 2 weeks, or at least about every 3 weeks. The amount of drug administered depends on the size of the tissue, the type of disease being treated, and the type of administration (e.g., local administration to a tissue in vivo using a system provided herein). Effective doses will vary, depending on the types of diseases treated, route of administration, excipient usage, and the possibility of co-usage with other therapeutic treatments.

### Efficacy

Therapeutic efficacy of an agent and/or pharmaceutical composition provided herein may be determined by evaluating and comparing patient symptoms and quality of life pre- and post-administration. Such methods apply irrespective of the mode of administration. In some embodiments, pre-administration refers to evaluating patient symptoms and quality of life prior to onset of therapy and post-administration refers to evaluating patient symptoms and quality of life at least 1, 2, 3, 4, 5, 6, 7, 8, 9, 10, 11, 12, 13, 14, 15, 16, 17, 18, 19, 20 weeks after onset of therapy. In a particular embodiment, the post-administration evaluating is performed about 2-8, 2-6, 4-6, or 4 weeks after onset of therapy. In a particular embodiment, patient symptoms (e.g., symptoms related to cancer, fibrosis, obesity, or autoimmune disease) and quality of life pre- and post-administration are evaluated clinically and by questionnaire assessment.

The agents and methods provided herein can be used to reduce cancer cell proliferation or survival in vivo or in vitro. Methods of evaluating tumor progression or cell proliferation are known in the art. In some embodiments, overall response is assessed from time-point response assessments (based on tumor burden) as follows:
Complete Response (CR): Disappearance of all target lesions. Any pathological lymph nodes (whether target or non-target) must have reduction in short axis to <10 mm.
Partial Response (PR): At least a 30% decrease in the sum of diameters of target lesions, taking as reference the baseline sum diameters.
Progressive Disease (PD): At least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm. (Note: the appearance of one or more new lesions is also considered progression).
Stable Disease (SD): Neither sufficient shrinkage to qualify for PR nor sufficient increase to qualify for PD, taking as reference the smallest sum diameters while on study.

In some embodiments, an in vitro cell proliferation assay is used to assess the efficacy of a one or more ferroptosis-inducing agents provided herein. The compositions and methods provided herein result in a reduction in the proliferation or survival of a plurality of cells. For example, after treatment with one or more of the agents provided herein, cell proliferation or survival is reduced by 5% or greater (e.g., 10%, 20%, 30%, 40%, 50%, 60%, 70%, 80%, 90% or greater) relative to cell proliferation or survival prior to treatment.

In some embodiments, animal models are used to assess the efficacy of a one or more ferroptosis-inducing agents ferroptosis-modulating agents provided herein in vivo. The ferroptosis-inducing agents and methods provided herein can result in a reduction in size or volume of a hyperproliferating tissue (e.g., a tumor). For example, after treatment, tissue size is reduced by 5% or greater (e.g., 10%, 20%, 30%, 40%, 50%, 60%, 70%, 80%, 90% or greater) relative to its size prior to treatment. Size of a tissue (e.g., a tumor) may be measured by any reproducible means of measurement. The size of a tissue may be measured as a diameter of the tumor or by any reproducible means of measurement. Ferroptosis inhibitors (e.g., an agent in Table 2) may be used to determine the efficacy of a particular test agent (also referred to herein as an active agent) for inducing ferroptosis in a tissue. For example, the combination of a ferroptosis inducer paired with a ferroptosis inhibitor (e.g., liproxstatin-1) can be used to determine whether the test agent targets a protein or nucleic acid involved in the ferroptosis pathway (see FIG. 1). Further provided herein is a method of rescuing a cell or plurality of cells from cell death and/or ferroptosis in vivo, the method comprising: administering to a subject a ferroptosis inhibitor. In some embodiments, the method further comprises administering a ferroptosis-inducing agent. Further provided herein is a method of screening a plurality of cells in a tissue for ferroptosis-sensitivity, the method comprising: contacting the tissue with a ferroptosis-inducing agent and a ferroptosis inhibitor; and measuring one or more parameters indicative of ferroptosis. In some embodiments, the ferroptosis-inducing agent is an agent in Table 1 or a test agent. In some embodiments, the ferroptosis inhibitor is any agent listed in Table 2. In some embodiments, the ferroptosis inhibitor is liproxstatin-1. In some embodiments, the one or more parameters indicative of ferroptosis are PUFA concentration, PI index, modulation of mesenchymal cell state marker expression, or modulation of iron or selenium concentration. The screening method provided herein can be readily scaled for high throughput analyses, that permit evaluation or prediction of the ferroptosis-inducing activity of test agents. Similarly, the screening method can be performed in animal models as discussed above in the presence and absence of a ferroptosis inhibitor.

Treating a disease or disorder (e.g., cancer, obesity, or a fibrotic disease) can further result in a decrease in number of hyperproliferative tissues and/or fat tissue (e.g., tumors or fat). For example, after treatment, hyperproliferative tissue or tumor number is reduced by 5% or greater (e.g., 10%, 20%, 30%, 40%, 50%, 60%, 70%, 80%, 90% or greater) relative to number prior to treatment. The number of tumors, cells, or tissues provided herein may be measured by any reproducible means of measurement. The number of tumors, cells, or tissues may be measured by counting tumors, cells, or tissues visible to the naked eye or at a specified magnification (e.g., 2×, 3×, 4×, 5×, 10×, or 50×). In some embodiments, methods and ferroptosis-inducing agents provided herein decrease the number of tumors in a subject.

In some embodiments, when administered to a subject in need thereof, ferroptosis-inducing agents or ferroptosis-modulating agents provided herein decrease the number of fat cells in a subject. In some cases, after treatment, there may be a decrease of about: 5%, 10%, 20%, 30%, 40%, 50%, 60%, 70%, 80%, 90% or greater of the number of adipose cells (fat cells) in a subject as to the amount before treatment. In some cases, the amount of adipose cells can decrease by about 5%-99%, 25-75% or 40-80% after treatment as compared to before treatment. In some cases, after treatment, there may be a decrease of about: 5%, 10%, 20%, 30%, 40%, 50%, 60%, 70%, 80%, 90% or greater of the amount of epididymal white adipose tissue in a subject as compared to before treatment. In some cases, the amount of epididymal white adipose tissue can decrease by about 5%-99%, 25-75% or 40-80% after treatment as compared to the amount before treatment. In some cases, the reduction can be determined by measuring the amount of epididymal white adipose tissue before and after the administration of the therapeutic amount of the ferroptosis-modulating agent.

In some cases, after treatment, there may be a decrease in the size of adipose cells. In some cases, the size of the adipose cells can decrease by about: 5%, 10%, 20%, 30%, 40%, 50%, 60%, 70%, 80%, 90% or greater after treatment as compared to the size before treatment. In some cases, the size of adipose cells can decrease by about 5%-99%, 25-75% or 40-80% after treatment as compared to the size before treatment. In some cases, the reduction can be determined by measuring the adipose cell size before and after the administration of the therapeutic amount of the ferroptosis-modulating agent.

In some embodiments, methods and ferroptosis-inducing agents provided herein increase the number or activity of leukocytes in a tumor microenvironment. In some embodiments, the leukocytes specifically target cancer cells with a high PUFA concentration as compared with normal cells.

Treating cancer can result in a decrease in number of metastatic nodules in other tissues or organs distant from the primary tumor site. For example, after treatment, the number of metastatic nodules is reduced by 5% or greater (e.g., 10%, 20%, 30%, 40%, 50%, 60%, 70%, 80%, 90% or greater) relative to number prior to treatment. The number of metastatic nodules may be measured by any reproducible means of measurement. The number of metastatic nodules may be measured by counting metastatic nodules visible to the naked eye or at a specified magnification (e.g., 2×, 10×, or 50×).

Treating a disease or disorder (e.g., cancer) can result in an increase in average survival time of a population of subjects treated according to the present invention in comparison to a population of untreated subjects. For example, the average survival time is increased by more than 30 days (more than 60 days, 90 days, 120 days or longer). An increase in average survival time of a population may be measured by any reproducible means. An increase in average survival time of a population may be measured, for example, by calculating for a population the average length of survival following initiation of treatment with the compound of the invention. An increase in average survival time of a population may also be measured, for example, by calculating for a population the average length of survival following completion of a first round of treatment with the compound of the invention.

Treating a disease or disorder (e.g., cancer) can also result in a decrease in the mortality rate of a population of treated subjects in comparison to an untreated population. For example, the mortality rate is decreased by more than 2% (e.g., more than 5%, 10%, 25%, or greater). A decrease in the mortality rate of a population of treated subjects may be measured by any reproducible means, for example, by calculating for a population the average number of disease-related deaths per unit time following initiation of treatment with the compound of the invention. A decrease in the mortality rate of a population may also be measured, for example, by calculating for a population the average number of disease-related deaths per unit time following completion of a first round of treatment with a ferroptosis-inducing agent.

Treating a disease or disorder can also result in a decrease in at least one symptom associated with the disease, disorder, or condition. In some embodiments, the methods provided herein reduce at least one symptom of a disease or disorder by at least 10%, 20%, 30%, 40%, 50%, 70%, 80%, 90% or greater relative to number prior to treatment. In some embodiments, following contact with a mammalian tissue or administration of a ferroptosis-inducing agent, cell death can be detected at a time point at or after contacting the mammalian tissue with the ferroptosis-inducing agent. In some embodiments, the methods provided herein increase cell death by at least 5%, 10%, 20%, 30%, 40%, 50%, 60%, 70%, 80%, 90% or greater relative to number prior to treatment.

### Therapeutic Applications

Provided herein are methods of treating a disease or a disorder in a subject. Also provided herein are compositions for use using any of the methods described herein. In some embodiments, the subject has, is suspected of having, or is at risk of developing a hyperproliferative disease or condition. In some embodiments, methods provided herein further comprise a step of obtaining a biopsy of the tissue for histological analysis. In some embodiments, the tissue comprises a histological abnormality, wherein the histological abnormality is hyperplasia or fibrosis.

Provided herein are methods of treating a disease such as obesity or overweight. In some cases, a method provided herein is inducing weight loss in a subject by administering a therapeutic amount of a ferroptosis-modulating agent. In some cases, the method can comprise reducing the adipose cell size of the subject. In some cases, the reduction is determined by measuring the adipose cell size before and after the administration of the therapeutic amount of the ferroptosis-modulating agent. In some cases, the method comprises reducing the epididymal white adipose tissue of the subject. In some cases, the reduction is determined by measuring the amount of the epididymal white adipose tissue before and after the administration of the therapeutic amount of the ferroptosis-modulating agent.

In some embodiments, the subject has, is suspected of having, or is at risk of developing a metabolic disease. In some embodiments, the metabolic disease is weight gain or obesity. In some embodiments, the subject has, is suspected of having, or is at risk of developing weight gain. In some embodiments, the subject is obese. In some cases, the subject has not been diagnosed with a cancer. In some embodiments, the subject is overweight. In some embodiments, the subject has, is suspected of having, or is at risk of developing a disease or condition associated with obesity. In some cases, the subject has a BMI of 25.0 to <30, which is overweight. In some cases, the subject has a BMI of 30.0 or higher, which is obese. In some cases, the subject can be Class 1 obese (BMI of 30 to <35), Class 2 obese (BMI of 35 to <40), or Class 3 three obese (BMI of 40 or higher).

In some embodiments, the subject can have, is suspected of having, is at risk of developing, or is diagnosed with diabetes type I, diabetes type II, diabetes type IIIa, or a metabolic syndrome.

In some instances, an agent or therapeutic or molecule can be a glucagon-like peptide 1 (GLP-1) agonist.

In some embodiments, the subject has, is suspected of having, or is at risk of developing a disease or condition associated with abnormal angiogenesis or vascularization. Diseases or conditions associated with abnormal angiogenesis or vascularization can include but are not limited to: ocular neovascularization, macular degeneration, retinopathy, sarcomas, polycystic kidney disease, benign hyperplasias, leiomyomas, adenomas, lipomas, hemangiomas, fibromas, vascular occlusion, restenosis, atherosclerosis, pre-neoplastic lesions, carcinoma in situ, and cancer. In some embodiments, the subject has, is suspected of having, or is at risk of developing an autoimmune disease. Non-limiting examples of relevant autoimmune diseases include: rheumatoid arthritis, inflammatory bowel disease, osteoarthritis, oral hairy leukoplakia, and psoriasis. In some embodiments, the subject has, is suspected of having, or is at risk of developing fibrosis. Non-limiting examples of diseases and conditions associated with fibrosis include: keloid scars, hypertrophic scars, systemic sclerosis, pulmonary arterial hypertension, cardiac fibrosis, hypertrophic cardiomyopathy valvular disease, myelofibrosis, myelodysplastic syndrome, chronic myelogenous leukemia, portal hypertension, hepatocellular carcinoma, retroperitoneal fibrosis, intestinal fibrosis, enteropathies, subretinal fibrosis, epiretinal fibrosis, cystic fibrosis, emphysema, pancreatic fibrosis, chronic pancreatitis, duct obstruction, arthrofibrosis, renal fibrosis, nephrogenic systemic fibrosis, renal anemia, chronic kidney disease, Dupuytren's disease, Ledderhose disease (plantar fibromatosis), primary biliary cholangitis (PBC), non-alcoholic steatohepatitis (NASH), scleroderma, diabetic neuropathy, hypertensive nephrosclerosis, allograft nephropathy, cirrhosis, and pulmonary fibrosis. In some embodiments, the subject has, is suspected of having, or is at risk of developing a disease or condition associated with a multicystic dsplastic kidney, fabry disease, cystinosis, glomerulonephritis, IgA nephropathy, lupus nephritis, atypical hemolytic uremic syndrome (aHUS), a nephrotic syndrome, kidney damage, polycystic kidney disease (PKD), renal artery stenosis, renal tubular acidosis, a simple kidney cyst, or a solitary or single-functioning kidney.

In some embodiments, the subject has, is suspected of having, or is at risk of developing cancer. In some embodiments, the subject has a benign tumor. In some embodiments, the subject has a pre-cancerous lesion. In some embodiments, the subject has a basal cell carcinoma (BCC) or a squamous cell carcinoma (SCC). In some embodiments, the subject has a metastatic tumor. In some embodiments, the cancer is a solid cancer or a blood cancer. In some embodiments, the blood cancer is a leukemia or a lymphoma. In some embodiments, the subject has a solid tumor. In some embodiments, the solid tumor is a carcinoma, a melanoma, or a sarcoma. In some embodiments, the melanoma is a dedifferentiated melanoma or amelanotic melanoma. In some embodiments, the subject has a melanoma with a B-Raf proto-oncogene, serine/threonine kinase (BRAF) mutation. In some embodiments the subject has a sarcoma with a Kirsten rat sarcoma (KRAS) mutation. In some embodiments, the sarcoma is a soft tissue sarcoma. In some embodiments, the sarcoma is leiomyosarcoma. In some embodiments, the carcinoma is a colon adenocarcinoma.

Non-limiting examples of cancer that can be treated with an agent provided herein include: acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B cell ALL, T cell ALL), acute myelocytic leukemia (AML) (e.g., B cell AML, T cell AML), chronic myelocytic leukemia (CML) (e.g., B cell CIVIL, T cell CML), and chronic lymphocytic leukemia (CLL) (e.g., B cell CLL, T cell CLL)); lymphoma such as Hodgkin lymphoma (HL) (e.g., B cell HL, T cell HL) and non-Hodgkin lymphoma (NHL) (e.g., B cell NHL such as diffuse large cell lymphoma (DLCL) (e.g., diffuse large B cell lymphoma), follicular lymphoma, chronic lymphocytic leukemia/small lymphocytic lymphoma (CLL/SLL), mantle cell lymphoma (MCL), marginal zone B cell lymphomas (e.g., mucosa associated lymphoid tissue (MALT) lymphomas, nodal marginal zone B cell lymphoma, splenic marginal zone B cell lymphoma), primary mediastinal B cell lymphoma, Burkitt lymphoma, lymphoplasmacytic lymphoma (e.g., Waldenstrom's macroglobulinemia), hairy cell leukemia (HCL), immunoblastic large cell lymphoma, precursor B lymphoblastic lymphoma and primary central nervous system (CNS) lymphoma; and T cell NHL such as precursor T lymphoblastic lymphoma/leukemia, peripheral T cell lymphoma (PTCL) (e.g., cutaneous T cell lymphoma (CTCL) (e.g., mycosis fungoides, Sezary syndrome), angioimmunoblastic T cell lymphoma, extranodal natural killer T cell lymphoma, enteropathy type T cell lymphoma, subcutaneous panniculitis like T cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), angiogenic myeloid metaplasia (AMM) a.k.a. myelofibrosis (MF), chronic idiopathic myelofibrosis, chronic myelocytic leukemia (CIVIL), chronic neutrophilic leukemia (CNL), hypereosinophilic syndrome (HES); neuroblastoma; neurofibroma (e.g., neurofibromatosis (NF) type 1 or type 2, schwannomatosis); neuroendocrine cancer (e.g., gastroenteropancreatic neuroendocrine tumor (GEP NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic adenocarcinoma, intraductal papillary mucinous neoplasm (IPMN), islet cell tumors); penile cancer (e.g., Paget's disease of the penis and scrotum); pinealoma; primitive neuroectodermal tumor (PNT); plasma cell neoplasia; paraneoplastic syndromes, intraepithelial neoplasms; prostate cancer (e.g., prostate adenocarcinoma); colorectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

Provided herein are methods of administering a ferroptosis-inducing agent to a tissue, wherein the tissue comprises different cell types. In some embodiments, the tissue comprises a heterogeneous population of cells, wherein the heterogeneous population of cells comprises at least one of precancerous cells and non-cancerous cells. In some embodiments, the tissue comprises a heterogeneous population of cells, wherein the heterogeneous population of cells comprises a population of immune cells.

Provided herein is a method of inducing immune cell recruitment to a tumor, the method comprising: administering to a subject a ferroptosis-inducing agent provided herein by any of the methods provided herein. In some embodiments, the administering is sustained administration for at least about 10 hours, thereby recruiting immune cells to the tumor site. In some embodiments, the immune cells are leukocytes. In some embodiments, following contact with a mammalian tissue or administration of a ferroptosis-inducing agent, immune cell recruitment can be detected at a time point at or after contacting the mammalian tissue with the ferroptosis-inducing agent. In some embodiments, the administering reduces the size of the tumor and/or increases the number of leukocytes within the tumor.

### Systems and Kits

Provided herein are systems for the delivery of a ferroptosis-inducing agent or an iron-dependent cell death inducing agent provided herein. Provided herein are systems for the delivery of a ferroptosis-modulating agent provided herein. Further provided herein are systems for inducing in vivo ferroptosis, the systems comprising: an implantable microdevice configured for localized administration to a tissue comprising: (a) a cylindrical support structure having at least one microwell on a surface of or formed within the support structure; (b) a microdose of a ferroptosis-inducing agent in the at least one microwell; and (c) a compound release mechanism for sustained administration for controlling a release of the ferroptosis-inducing agent from the microwell, wherein the microdose of the ferroptosis-inducing agent forms a gradient of a sub-therapeutic dose of the ferroptosis-inducing agent an administration site within the tissue for a duration of time of at least 4 hours, wherein the microdevice is configured to permit implantation into the tissue using a catheter, cannula or biopsy needle, and wherein the microdevice is further configured to release the ferroptosis-inducing agent from the at least one microwell to the administration site within the apoptosis-resistant tissue adjacent to the at least one microwell.

Further provided herein are systems for identifying ferroptosis induction in an animal model comprising: (a) an animal model comprising a target tissue of interest; (b) a microdevice configured to permit implantation into a tissue in the animal model using a catheter, cannula or biopsy needle comprising: (i) at least one microwell containing one or more active agents; (ii) a micro-dose of the one or more active agents in the at least one microwell; and (iii) a compound release mechanism comprising a polymeric matrix for controlling the release of the one or more active agents from the microwell into the tissue; wherein the system measures an outcome of ferroptosis induction in the animal model after administration of the one or more active agents into the tissue relative to a baseline tissue without administration of the one or more active agents, and identifying one or more active agents induces ferroptosis in the tissue.

Further provided herein are systems for screening for ferroptosis-induced cell death in vivo, the systems comprising: (a) an animal model comprising a target tissue of interest; (b) a microdevice configured to permit implantation into a tissue in the animal model using a catheter, cannula or biopsy needle comprising: (i) at least one microwell containing one or more active agents; (ii) at least one microwell containing one or more ferroptosis inhibitors; (ii) a micro-dose of the one or more active agents; and/or one or more ferroptosis inhibitors in the at least one microwell; and (iii) a compound release mechanism comprising a polymeric matrix for controlling the release of the one or more active agents from the microwell into the tissue; wherein the system measures an outcome of ferroptosis induction in the animal model after administration of the one or more active agents into the tissue relative to a baseline tissue without administration of the one or more active agents, wherein the system measures an outcome of ferroptosis induction in the animal model after administration of the one or more active agents into the tissue relative to administration of the one or more active agents and one or more ferroptosis inhibitors, and identifying one or more active agents induces ferroptosis in the tissue.

The systems provided herein generally include multiple microwells arranged on or within a support structure. The microwells contain one or more active agents, alone or in combination, in one or more dosages and/or release pharmacokinetics. Preferably, the devices are configured to deliver the microdose amounts so as to virtually eliminate overlap in the tissue of active agents released from different microwells. In some embodiments, the devices are configured to facilitate implantation and retrieval in a target tissue. In an exemplary embodiment, the device has a cylindrical shape, having symmetrical wells on the outside of the device, each well containing one or more drugs, at one or more concentrations. The device is sized to permit placement using a catheter, cannula, or stylet. In a preferred embodiment, the device has a guidewire to assist in placement and retrieval. The device may also include features that assist in maintaining spatial stability of tissue excised with the device, such as fins or stabilizers that can be expanded from the device prior to or at the time of removal. Optionally, the device has fiber optics, sensors and/or interactive features such as remote accessibility (such as Wi-Fi) to provide for in situ retrieval of information and modification of device release properties. In the most preferred embodiment, the fiber optics and/or sensors are individually accessible to discrete wells.

In some embodiments, the systems provided herein are formed of biocompatible silicon, metal, ceramic or polymers. They may include materials such as radiopaque materials or materials that can be imaged using ultrasound or MRI. They can be manufactured using techniques such as deep ion etching, nano imprint lithography, micromachining, laser etching, three-dimensional printing or stereolithography. Drug can be loaded by injection of a solution or suspension into the wells followed by solvent removal by drying, evaporation, or lyophilization, or by placement of drug in tablet or particulate form into the wells. In a preferred embodiment, drugs are loaded on top of hydrogel pads within the microwells. The hydrogel pads expand during implantation to deliver the drugs to the surrounding tissue. Drug release pharmacokinetics are a function of drug solubility, excipients, dimensions of the wells, and tissue into which the device is implanted (with greater rate of release into more highly vascularized tissue, than into less vascular tissue).

In some embodiments, the systems provided herein are implanted directly into a solid tumor or tissue to be biopsied. Upon implantation, the systems provided herein locally release an array of active agents in microdoses. Subsequent analysis of tumor response to the array of active agents can be used to identify particular drugs, combinations of drugs, and/or dosages that are effective for treating a solid tumor in a patient. By locally delivering microdoses of an array of drugs, the microassay device can be used to test patients for response to large range of regimens, without inducing systemic toxicities, quickly and under actual physiological conditions. These data are used, optionally in combination with genomic data, to accurately predict systemic drug response.

Without limitation, the systems provided herein can administer an agent provided herein according to any of the methods provided herein. For example, a system provided herein can be used to deliver a microdose of an agent to a tissue in vivo. The systems described herein can provide sustained administration of a therapeutic amount of a ferroptosis-inducing agent to a tissue, wherein the sustained administration of said therapeutic amount comprises providing to said tissue the ferroptosis-inducing agent in an amount sufficient to achieve a distribution of at least about 10 ng/mm² within said tissue for a period of at least 4 hours, thereby inducing ferroptosis in the tissue. In some embodiments, the sustained administration further forms a gradient of a sub-therapeutic amount of the ferroptosis-inducing agent adjacent to the administration site within the tissue. In some embodiments, the sustained administration of a therapeutic amount of a ferroptosis-inducing agent is at least 10 hours. In some embodiments, the therapeutic amount of a ferroptosis-inducing agent is a concentration of at least about 1 μM up to 10 μM. In some embodiments, a system provided herein is implanted into a tumor. In some embodiments, the system delivers one or more a ferroptosis-inducing agents to a tumor.

Provided herein are kits comprising a ferroptosis-modulating agent or a ferroptosis-inducing agent. In some cases, the kits herein can be in a container. In some cases, the container can comprise glass, plastic, metal or a combination thereof.

### Exemplary Embodiments

A number of compositions, and methods are disclosed herein. Specific exemplary embodiments of these compositions and methods are disclosed below. The following embodiments recite non-limiting permutations of combinations of features disclosed herein. Other permutations of combinations of features are also contemplated. In particular, each of these numbered embodiments is contemplated as depending from or relating to every previous or subsequent numbered embodiment, independent of their order as listed.

Embodiment 1. A method of inducing weight-loss or fat loss in a subject, comprising administering to the subject a ferroptosis inducing agent in an amount sufficient to reduce adipose cell size, or number of adipose cells in the subject.

Embodiment 2. The method of embodiment 1, wherein the subject is human.

Embodiment 3. The method of embodiment 1, wherein the ferroptosis inducing agent is administered orally.

Embodiment 4. The method of embodiment 1, wherein the administering is discontinuous and the administration occurs daily for about: 5 days, 10 days, 15 days, 20, days, 25 days, 30 days, two months, three months, four months, five months, six months, or one year.

Embodiment 5. The method of embodiment 1, wherein the administration results in reducing an adipose cell size, or number of adipose cells of the subject, wherein the reduction is determined by measuring the adipose cell size, or number of adipose cells, before and after the administration of the therapeutic amount of the ferroptosis-modulating agent, optionally employing a microscopic evaluation of a biopsy.

Embodiment 6 The method of embodiment 1, wherein the method further comprises reducing an epididymal white adipose tissue of the subject.

Embodiment 7. The method of embodiment 6, wherein the subject is overweight or obese.

Embodiment 8. The method of embodiment 6, wherein the subject is obese and has a BMI of 30.0 or higher.

Embodiment 9. The method of embodiment 6, wherein the subject is overweight and has a BMI of 25.0 to <30.

Embodiment 10. A method of inducing, regulating, or modulating weight loss, body composition, or fat loss or fat reduction in a subject, the method comprising administering to the subject a therapeutically effective amount of a ferroptosis-modulating agent, a glutamate-cysteine ligase (GCL) modulating agent, an agent that binds to GCL, an agent that inhibits GCL, or any combination thereof to the subject, thereby inducing, regulating, or modulating the weight loss, body composition, or fat loss or fat reduction in the subject.

Embodiment 11. The method of embodiment 10, wherein the subject is a human.

Embodiment 12. The method of embodiment 11, wherein the subject is a male.

Embodiment 13. The method of any preceding embodiment, wherein the agent is delivered orally as a solution having a concentration of at least about: 5 mg agent per mL of the solution.

Embodiment 14. The method of embodiment 1, wherein the administration is discontinuous and the administration occurs daily for about: 5 days, 10 days, 15 days, 20, days, 25 days, 30 days, two months, three months, four months, five months, six months, or one year.

Embodiment 15. The method of embodiment 1, wherein the agent is selected from the group consisting of: (1S,3R)-RSL3, ML-162, ML-210, JKE-1674, JKE-1716, erastin, jacaric acid, trigonelline, glutamate, sulfasalazine, auranofin, brusatol, sorafenib, sorafenib-d3, sorafenib tosylate, trigonelline, FIN56, FINO2, CIL56, dihydroisotanshinone I, GPX4-IN-3, a sulfoximine, BSO, and a salt of any of these.

Embodiment 16. The method of embodiment 1, wherein the administration results in reducing an adipose cell size of the subject, wherein the reduction is determined by measuring the adipose cell size before and after the administration of the therapeutic amount of the ferroptosis-modulating agent, optionally employing a microscopic evaluation of a biopsy.

Embodiment 17. The method of embodiment 1, wherein the administration results in reducing the number of adipose cells of the subject, wherein the reduction is determined by measuring the number of adipose cells before and after the administration of the therapeutic amount of the ferroptosis-modulating agent, optionally employing a microscopic evaluation of a biopsy.

Embodiment 18. The method of embodiment 16 or embodiment 17, wherein the method comprises reducing an epididymal white adipose tissue of the subject.

Embodiment 19. The method of embodiment 18, wherein the subject is overweight or obese.

Embodiment 20. The method of embodiment 19, wherein the subject is obese and has a BMI of 30.0 or higher.

Embodiment 21. The method of embodiment 19, wherein the subject is overweight and has a BMI of 25.0 to <30.

Embodiment 22. The method of any preceding embodiment, wherein the subject is administered concurrently or consecutively an additional agent or therapy.

Embodiment 23. The method of embodiment 22, wherein the additional agent or therapy is selected from the group consisting of: a GLP-1 agonist, a gastric inhibitory polypeptide analog, naltrexone-bupropion or a salt thereof, orlistat or a salt thereof, phentermine or a salt thereof, phentermine-topiramate or a salt thereof, selenium, an exercise therapy, a surgery, a diet modification, and any combination thereof.

Embodiment 24. The method of embodiment 23, wherein the additional agent or therapy comprises administering the GLP-1 agonist that comprises dulaglutide, exenatide, semaglutide, liraglutide, lixisenatide, or a salt of any of these.

Embodiment 25. The method of embodiment 23, wherein the additional agent or therapy comprises administering the gastric inhibitory polypeptide that comprises tirzepatide or a derivative thereof.

Embodiment 26. The method of embodiment 23, wherein additional agent or therapy comprises the surgery that comprises a gastric sleeve, a gastric bypass, an adjustable gastric band, a ball that is placed in the stomach, or any combination thereof.

Embodiment 27. The method of any preceding embodiment, wherein the subject has not been diagnosed with a cancer.

Embodiment 28. The method of any preceding embodiment, wherein the subject is a subject in need thereof.

Embodiment 29. The method of any preceding embodiment, wherein the subject has diabetes type I, diabetes type II, diabetes type IIIa, or a metabolic syndrome.

Embodiment 30. The method of any preceding embodiment, wherein the agent is in the form of a pharmaceutical composition that further comprises a pharmaceutically acceptable: carrier, excipient, or diluent.

Embodiment 31. The method of embodiment 30, wherein the pharmaceutical composition is in unit dose form.

Embodiment 32. A composition comprising i) a ferroptosis-modulating agent, a glutamate-cysteine ligase (GCL) modulating agent, an agent that binds to GCL, an agent that inhibits GCL; and ii) a GLP-1 agonist or a salt thereof, a gastric inhibitory polypeptide analog, a naltrexone-bupropion or a salt thereof, an orlistat or a salt thereof, a phentermine or a salt thereof, and a phentermine-topiramate or a salt thereof, or a selenium or a salt thereof.

Embodiment 33. A composition comprising i) a glutamate-cysteine ligase (GCL) modulating agent, and ii) a GLP-1 agonist or a salt thereof, a gastric inhibitory polypeptide analog, a naltrexone-bupropion or a salt thereof, an orlistat or a salt thereof, a phentermine or a salt thereof, and a phentermine-topiramate or a salt thereof, or a selenium or a salt thereof.

Embodiment 34. The composition of embodiment 32 or embodiment 33, which is a pharmaceutical composition.

Embodiment 35. The pharmaceutical composition of embodiment 34, that is in unit dose form.

Embodiment 36. The composition of any one of embodiments 32-35, for use in the treatment of a disease or condition.

Embodiment 37. The composition of any one of embodiments 32-35, for use in the treatment of a kidney disease or condition.

Embodiment 38. The composition of any one of embodiments 32-35, for use in the treatment of a symptom associated with a kidney disease or condition.

Embodiment 39. The composition for use of embodiment 36, wherein the disease or condition is weight loss, weight maintenance, an iron metabolism associated disease or condition, or a disease or condition associated with high, low, or abnormal cholesterol.

Embodiment 40. A method of modulating iron metabolism in a subject, the method comprising administering to the subject a therapeutically effective amount or an effective amount of a ferroptosis-inducing agent, a glutamate-cysteine ligase (GCL) modulating agent, an agent that binds to GCL, an agent that inhibits GCL, or any combination thereof, thereby modulating the iron metabolism in the subject and thereby inducing ferroptosis in the subject.

Embodiment 41. A method of modulating iron metabolism in a subject, the method comprising administering to the subject a therapeutically effective amount or an effective amount of a ferroptosis-inducing agent, thereby modulating the iron metabolism in the subject.

Embodiment 42. A method of treating, inducing, regulating, or modulating iron metabolism or a disease or condition associated with iron metabolism in a subject, the method comprising administering to the subject a therapeutically effective amount or an effective amount of a ferroptosis-modulating agent, a glutamate-cysteine ligase (GCL) modulating agent, an agent that binds to GCL, an agent that inhibits GCL, or any combination thereof, thereby treating, inducing, regulating, or modulating the iron metabolism or the disease or condition associated with the iron metabolism in the subject.

Embodiment 43. A method of modulating cholesterol metabolism in a subject, the method comprising administering to the subject a therapeutically effective amount or an effective amount of a ferroptosis-modulating agent, a glutamate-cysteine ligase (GCL) modulating agent, an agent that binds to GCL, an agent that inhibits GCL, or any combination thereof, thereby modulating cholesterol metabolism in the subject.

Embodiment 44. A method of modulating cholesterol metabolism in a subject, the method comprising administering to the subject a therapeutically effective amount or an effective amount of a ferroptosis-inducing agent, thereby modulating the cholesterol metabolism in the subject.

Embodiment 45. A method of treating, inducing, regulating, modulating, or diminishing cholesterol metabolism or a disease associated with cholesterol metabolism in a subject, the method comprising administering to the subject a therapeutically effective amount or an effective amount of a ferroptosis-modulating agent, a glutamate-cysteine ligase (GCL) modulating agent, an agent that binds to GCL, an agent that inhibits GCL, or any combination thereof, thereby treating, inducing, regulating, modulating, or diminishing the cholesterol metabolism or the disease or condition associated with the cholesterol metabolism in the subject.

Embodiment 46. A method of treating, inducing, regulating, modulating, diminishing, or increasing a reactive oxygen species or a disease or condition associated with a reactive oxygen species in a subject, the method comprising administering to the subject a therapeutically effective amount or an effective amount of a ferroptosis-modulating agent, a glutamate-cysteine ligase (GCL) modulating agent, an agent that binds to GCL, an agent that inhibits GCL, or any combination thereof, thereby treating, inducing, regulating, modulating, diminishing, or increasing the reactive oxygen species or the disease or condition associated with the reactive oxygen species in the subject.

Embodiment 47. A method of increasing a reactive oxygen species in a subject, the method comprising administering to the subject a therapeutically effective amount or an effective amount of a ferroptosis-modulating agent, a glutamate-cysteine ligase (GCL) modulating agent, an agent that binds to GCL, an agent that inhibits GCL, or any combination thereof, thereby increasing the reactive oxygen species in the subject.

Embodiment 48. A method of promoting at least one of: treating, surviving, modulating, or diminishing an acute stress; a chronic stress response; an immune hypersensitivity; cachexia; kidney disease; neurodegeneration; cardiotoxicity; or cardiotoxicity caused at least in part by chemotherapy; in a subject, the method comprising administering to the subject a therapeutically effective amount or an effective amount of a ferroptosis-modulating agent, a glutamate-cysteine ligase (GCL) modulating agent, an agent that binds to GCL, an agent that inhibits GCL, or any combination thereof, thereby treating, inducing, regulating, modulating, diminishing, or increasing the acute stress; a chronic stress response; an immune hypersensitivity; cachexia; kidney disease; neurodegeneration; cardiotoxicity; or cardiotoxicity cause at least in part by chemotherapy in the subject.

Embodiment 49. A method of promoting at least one of: treating a kidney disease in a subject, the method comprising administering to the subject a therapeutically effective amount or an effective amount of a ferroptosis-modulating agent, a glutamate-cysteine ligase (GCL) modulating agent, an agent that binds to GCL, an agent that inhibits GCL, or any combination thereof, thereby treating the kidney disease in the subject.

Embodiment 50. A method of promoting at least one of: treating a kidney disease in a subject, the method comprising administering to the subject a therapeutically effective amount or an effective amount of a ferroptosis-inducing agent, thereby treating the kidney disease in the subject.

Embodiment 51. The method of any one of embodiments 40-50, wherein the method comprises reducing an epididymal white adipose tissue of the subject.

Embodiment 52. The method of embodiment 51, wherein the subject is overweight or obese.

Embodiment 53. The method of embodiment 52, wherein the subject is obese and has a BMI of 30.0 or higher.

Embodiment 54. The method of embodiment 52, wherein the subject is overweight and has a BMI of 25.0 to <30.

Embodiment 55. The method of any one of embodiments 40-54, wherein the subject is administered concurrently or consecutively an additional agent or therapy.

Embodiment 56. The method of embodiment 55, wherein the additional agent or therapy is selected from the group consisting of: a GLP-1 agonist, a gastric inhibitory polypeptide analog, naltrexone-bupropion or a salt thereof, orlistat or a salt thereof, phentermine or a salt thereof, phentermine-topiramate or a salt thereof, selenium, an exercise therapy, a surgery, a diet modification, and any combination thereof.

Embodiment 57. The method of embodiment 56, wherein the additional agent or therapy comprises administering the GLP-1 agonist that comprises dulaglutide, exenatide, semaglutide, liraglutide, lixisenatide, or a salt of any of these.

Embodiment 58. The method of embodiment 56, wherein the additional agent or therapy comprises administering the gastric inhibitory polypeptide that comprises tirzepatide or a derivative thereof.

Embodiment 59. The method of embodiment 56, wherein additional agent or therapy comprises the surgery that comprises a gastric sleeve, a gastric bypass, an adjustable gastric band, a ball that is placed in the stomach, or any combination thereof.

Embodiment 60. The method of any one of embodiments 40-59, wherein the subject has not been diagnosed with a cancer.

Embodiment 61. The method of any one of embodiments 40-59, wherein the subject is a subject in need thereof.

Embodiment 62. The method of any one of embodiments 40-59, wherein the subject has diabetes type I, diabetes type II, diabetes type Illa, or a metabolic syndrome.

Embodiment 63. The method of any one of embodiments 40-59, wherein the agent is in the form of a pharmaceutical composition that further comprises a pharmaceutically acceptable: carrier, excipient, or diluent.

Embodiment 64. The method of embodiment 63, wherein the pharmaceutical composition is in unit dose form.

Embodiment 65. The method of any preceding embodiment, wherein the agent is a compound or a salt thereof in Table 1.

Embodiment 66. The method of any preceding embodiment, wherein the agent is in the form of a pharmaceutical composition that further comprises a pharmaceutically acceptable: carrier, excipient, or diluent.

Embodiment 67. The method of embodiment 46, wherein the pharmaceutical composition is in unit dose form.

Embodiment 68. A composition comprising i) a ferroptosis-modulating agent, a glutamate-cysteine ligase (GCL) modulating agent, an agent that binds to GCL, an agent that inhibits GCL; and ii) a GLP-1 agonist or a salt thereof, a gastric inhibitory polypeptide analog, a naltrexone-bupropion or a salt thereof, an orlistat or a salt thereof, a phentermine or a salt thereof, and a phentermine-topiramate or a salt thereof, or a selenium or a salt thereof.

Embodiment 69. The composition of embodiment 68, which is a pharmaceutical composition.

Embodiment 70. The pharmaceutical composition of embodiment 69, that is in unit dose form.

Embodiment 71. The composition of any one of embodiments 68-70, for use in the treatment of a disease or condition.

Embodiment 72. The composition of any one of embodiments 68-70, for use in the treatment of a kidney disease or condition.

Embodiment 73. The composition of any one of embodiments 68-70, for use in the treatment of a symptom associated with a kidney disease or condition.

Embodiment 74. The composition for use of embodiment 71, wherein the disease or condition is weight loss, weight maintenance, an iron metabolism associated disease or condition, or a disease or condition associated with high, low, or abnormal cholesterol.

Embodiment 75. A composition for use for inducing weight loss, fat loss, or both, in a subject, the composition for use comprising inducing agent in an amount sufficient to induce the weight loss, the fat loss, or both.

Embodiment 76. The composition for use of embodiment 75, for inducing weight loss.

Embodiment 77. The composition of use of embodiment 75, for inducing fat loss.

Embodiment 78. The composition for use of embodiment 75, for inducing weight loss and fat loss.

Embodiment 79. The composition for use of embodiment 75, further comprising, before the administering to the subject the ferroptosis inducing agent, administering a priming agent to the subject.

Embodiment 80. The composition for use of embodiment 75, wherein an adipose tissue in the subject, after the administrating of the ferroptosis inducing agent to the subject, diminishes by weight, by volume, or both, relative to a weight, volume, or both of the adipose tissue in the subject before the administering.

Embodiment 81. The composition for use of embodiment 80, wherein the adipose tissue in the subject, after the administering to the subject of the ferroptosis inducing agent, diminishes by volume relative to a volume of the adipose tissue before the administering, as determined by a pinch clamp test, by a dual x-ray absorptiometry (DEXA) scan, a diminished waist measurement, a computerized axial tomography (CAT) test, or any combination thereof.

Embodiment 82. The composition for use of embodiment 80, wherein the adipose tissue in the subject, after the administering to the subject of the ferroptosis inducing agent, diminishes by weight relative to a weight of the adipose tissue before the administering.

Embodiment 83. The composition for use of embodiment 82, wherein the diminishes by weight is determined by the subject weighing less in total body weight after the administering of the ferroptosis inducing agent as compared to the total body weight of the subject before the administering of the ferroptosis inducing agent.

Embodiment 84. The composition for use of embodiment 82, wherein the diminishes by weight is determined by comparing DEXA scan results of the subject taken before and after the administering to the subject of a ferroptosis inducing agent.

Embodiment 85. The composition for use of embodiment 75, further comprising that a cell size, a cell volume, or both, of a plurality of cells in an adipose tissue of the subject is diminished after the administering to the subject of a ferroptosis inducing agent, relative to a cell size, a cell volume, or both, of a plurality of cells in the adipose tissue of the subject before the administering.

Embodiment 86. The composition for use of embodiment 75, wherein the subject is a human.

Embodiment 87. The composition for use of embodiment 75, wherein the ferroptosis inducing agent is administered orally.

Embodiment 88. The composition for use of embodiment 75, wherein the administration occurs for about: 1 day, 2 days, 3 days, 4 days, 5 days, one week, 10 days, two weeks, 15 days, 20, days, three weeks, 25 days, 30 days, one month, two months, three months, four months, five months, six months, one year, two years, three years, four years, five years, for life, or as needed.

Embodiment 89. The composition for use of embodiment 87, wherein the ferroptosis inducing agent is administered in an amount ranging from about 0.1 ng to about 25,000 mg.

Embodiment 90. The composition for use of embodiment 87, wherein the ferroptosis inducing agent is administered in an amount of about: 1 ng, 10 ng, 100 ng, 1 microgram, 10 micrograms, 100 micrograms, 1 mg, 10 mg, 100 mg, 1 g, 2 g, 3 g, 4 g, 5 g, 6 g, 7 g, 8 g, 9 g, 10 g, 15 g, 20 g, or 25 g.

Embodiment 91. The composition for use of embodiment 87, wherein the ferroptosis inducing agent is administered in an amount ranging from about 1 ng/kg to 1,000 mg/kg, wherein mg is mg of the ferroptosis inducing agent, and kg is kg of body weight of the subject.

Embodiment 92. The composition for use of embodiment 75, wherein the ferroptosis inducing agent is administered orally.

Embodiment 93. The composition for use of embodiment 75, wherein the ferroptosis inducing agent is administered by one of the following routes: oral administration, subcutaneous, intradermal, intramuscular, inhalation, intravenous, intraperitoneal, intracranial, intrathecal, transcutaneous, rectal or directly to a fatty tissue.

Embodiment 94. The composition for use of embodiment 75, that treats at least one of: obesity, metabolic syndrome, elevated blood glucose, a diabetes, diabetes type 2, diabetes type 3, insulin resistance, high blood pressure, a cardiovascular disease, a coronary artery disease, a cerebrovascular disease, a stroke, a rheumatic heart disease, an arteriosclerosis, an atherosclerosis, a liver disease, a fatty liver disease, a nonalcoholic fatty liver disease (NAFLD), a nonalcoholic steatohepatitis (NASH), or any combination thereof.

Embodiment 95. The composition for use of embodiment 75, wherein the subject is a human male.

Embodiment 96. The composition for use of embodiment 75, wherein the subject is a human female.

Embodiment 97. The composition for use of embodiment 75, wherein the subject ranges from about 1 year of age to about 18 years of age.

Embodiment 98. The composition for use of embodiment 75, wherein the subject ranges from about 18 years of age to about 120 years of age.

Embodiment 99. The composition for use of embodiment 75, wherein the subject is a subject in need thereof.

Embodiment 100. The composition for use of embodiment 75, wherein the ferroptosis inducing agent is comprised in a pharmaceutical composition that comprises a pharmaceutically acceptable: excipient, carrier, or diluent.

Embodiment 101. The composition for use of embodiment 100, wherein the pharmaceutical composition is in unit dose form.

Embodiment 102. The composition for use of embodiment 75, further comprising administering a further therapeutic to the subject.

Embodiment 103. The composition for use of embodiment 102, wherein the further therapeutic is administered concurrently with the ferroptosis inducing agent.

Embodiment 104. The composition for use of embodiment 102, wherein the further therapeutic is administered consecutively with the ferroptosis inducing agent.

Embodiment 105. The composition for use of embodiment 75, wherein the ferroptosis inducing agent is administered directly to a fatty tissue Embodiment 106. The composition for use of embodiment 105, wherein the administering is continuously.

Embodiment 107. The composition for use of embodiment 105, wherein the administering is for about: ½ hour, 1 hour, 2 hours, 3 hours, four hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 2 days, 3 days, 4 days, 5 days, 6 days, or one week.

Embodiment 108. The composition for use of embodiment 105, wherein the fatty tissue is heated to a temperature of about; 23, 24, 25, 26, 27, 28, 29, 30, 31, 32, or 33 degrees Celsius.

Embodiment 109. The composition for use of embodiment 105, wherein the ferroptosis inducing agent is administered in the form of an aqueous solution.

Embodiment 110. The composition for use of embodiment 105, wherein a concentration of the ferroptosis inducing agent in the aqueous solution is from about 0.1 nM to about 500 microM.

Embodiment 111. The composition for use of embodiment 75, wherein the administration results in reducing an adipose cell size, or number of adipose cells of the subject, wherein the reduction is determined by measuring the adipose cell size, or number of adipose cells, before and after the administration of the therapeutic amount of the ferroptosis-modulating agent, optionally employing a microscopic evaluation of a biopsy.

Embodiment 112. The composition for use of embodiment 75, wherein the method further comprises reducing an epididymal white adipose tissue of the subject.

Embodiment 113. The composition for use of embodiment 75, wherein the subject is overweight or obese.

Embodiment 114. The composition for use of embodiment 75, wherein the subject is obese and has a BMI of about 30.0 or higher.

Embodiment 115. The composition for use of embodiment 75, wherein the subject is overweight and has a BMI of about 25.0 to about 30.

Embodiment 116. The composition for use of embodiment 102, wherein the further therapeutic agent has glucagon-like peptide-1 (GLP-1) receptor agonist activity.

Embodiment 117. The composition for use of embodiment 116, wherein the further therapeutic agent comprises semaglutide, dulaglutide, liraglutide, exenatide, tirzepatide, or any combination thereof.

### Examples

The following examples are set forth to illustrate more clearly the principle and practice of embodiments disclosed herein to those skilled in the art and are not to be construed as limiting the scope of any claimed embodiments. Unless otherwise stated, all parts and percentages are on a weight basis.

### Example 1: Cell Lines and Culture Conditions

Human cancer cell lines are cultured in Ham's F12 medium supplemented with 10% (v/v) fetal bovine serum (FBS), penicillin (100 U/mL), and streptomycin (100 μg/mL). human cancer cells are cultured in RPMI medium supplemented with 10% FBS, penicillin (100 U/mL), and streptomycin (100 μg/mL). Cells are grown in a humidified incubator at 37° C. with 5% carbon dioxide and split every 3-4 days using trypsin/EDTA solution.

Method to add exogenous PUFAs or MUFAs: Exogenous fatty acids were dissolved in DMSO and added to cell culture medium 24 h after seeding cells.

### Example 2: Determination of PUFA Status

Lipidomics are performed using either gas chromatography-mass spectrometry (GC-MS) or direct infusion mass spectrometry.

For GC-MS assessment of cellular PUFA status, the membrane lipids are trans-esterified with 500 μL methanolic HCl, 250 μL n-hexane and 500 μL internal standard (0.8 mg Di-C17-phosphatidylcholine in 1 mL methanol with 0.2% Butylhydroxytoluol as antioxidant). After cooling-off, 500 μL n-hexane and 1 mL Aqua Dest. are added. The upper hexane phase is evaporated with nitrogen. The fatty acid methylesters (FAME) are taken up in 60 μL n-hexane. An aliquot of 1 μL is injected on-column on a Varian CP 3800 gas chromatograph (Varian, Darmstadt, Germany) equipped with an Omegawax™ 320 column (0.32 mm internal diameter, 30 m length) (Supelco, Bellefonte, USA). The column temperature was 200 degrees C.

For direct infusion MS analysis, lipids are extracted using a two-step chloroform/methanol procedure. Samples are spiked with internal lipid standard mixture containing: cardiolipin 16:1/15:0/15:0/15:0 (CL), ceramide 18:1;2/17:0 (Cer), diacylglycerol 17:0/17:0 (DAG), hexosylceramide 18:1;2/12:0 (HexCer), lyso-phosphatidate 17:0 (LPA), lyso-phosphatidylcholine 12:0 (LPC), lyso-phosphatidylethanolamine 17:1 (LPE), lyso-phosphatidylglycerol 17:1 (LPG), lyso-phosphatidylinositol 17:1 (LPI), lyso-phosphatidylserine 17:1 (LPS), phosphatidate 17:0/17:0 (PA), phosphatidylcholine 17:0/17:0 (PC), phosphatidylethanolamine 17:0/17:0 (PE), phosphatidylglycerol 17:0/17:0 (PG), phosphatidylinositol 16:0/16:0 (PI), phosphatidylserine 17:0/17:0 (PS), cholesterol ester 20:0 (CE), sphingomyelin 18:1;2/12:0;0 (SM), triacylglycerol 17:0/17:0/17:0 (TAG). After extraction, the organic phase is transferred to an infusion plate and dried in a speed vacuum concentrator. The dried extract is re-suspended in 7.5 mM ammonium acetate in chloroform/methanol/propanol (1:2:4, V:V:V) and the second step dry extract is re-suspended in a 33% ethanol solution of methylamine in chloroform/methanol (0.003:5:1; V:V:V). Samples are analyzed by direct infusion on a QExactive mass spectrometer (ThermoFisher Scientific) equipped with a TriVersa NanoMate ion source (Advion Biosciences). Samples are analyzed in both positive and negative ion modes with a resolution of Rm/z=200=280000 for MS and Rm/z=200=17500 for tandem mass spectrometry (MS-MS) assays, in a single acquisition. MS-MS is triggered by an inclusion list encompassing corresponding MS mass ranges scanned in 1 Da increments.

Example 3: Cell Line Profiling with a Ferroptosis-Inducing Agent with and without a Rescue Agent

Cell viability assays are performed by seeding 1,000 cells per well (30 μL volume) in opaque white 384-well plates (Corning). Cells are allowed to adhere for 24 h, after which they are exposed to compounds for 72 hours. DMSO stock solutions of compounds are added to cells using a CyBio Well Vario liquid dispenser (Analytik Jena AG). Cellular ATP levels are measured using CellTiter-Glo (Promega) as a surrogate for viability. Rescue assays are performed using rescue agents selected from Table 2 and referred to in the assays as anti-ferroptosis rescue agent (N) (N, 1.5 μM), anti-ferroptosis rescue agent (M) (M, 1 μM), anti-ferroptosis rescue agent (P) (P, 50 μM), and other ferroptosis inhibitors added to cells at the time of addition to assay plates.

### Knocking Down Targets Using Genetic Reagents+/−Anti-Ferroptosis Rescue Agent (N)

For lentiviral shRNA production, 293-T cells are seeded in 6-well dishes in antibiotic free media (280,000 cells/well). The next day, cells were transfected using FuGENE with the appropriate shRNA encoding plasmid (450 ng), viral packaging plasmid (p-Delta8.9, 400 ng), and viral envelope plasmid (p-VSV-G, 45 ng). After 24 h, the medium is removed and replaced with fresh medium. Three collections of viral supernatant per shRNA are made over 36 h and pooled. The combined supernatant is centrifuged, aliquoted, and stored at −80 degrees C. until virus infection.

Lentiviral infections are performed by seeding cells for 12 h and replacing the media with media supplemented with polybrene (8 μg/mL) and an aliquot of the viral supernatant. Plates are incubated for 48 h and the media is replaced with media containing 1.5 μg/mL puromycin and incubated at 37 degrees C. for 48 h. Knockdown is assessed by immunoblotting and RT-qPCR.

### Knocking Out Targets Using Genetic Reagents+/−Anti-Ferroptosis Rescue Agent (N)

For generation of cell lines with gene knockouts, lentiviruses are generated by overnight polyethylenimine transfection of Leni-X 293T cells with target lentiviral plasmid and packaging plasmids pCMV-dR8.2 dvpr and pCMV-VSV-G in DMEM supplemented with 10% FBS. The next day, the medium is changed to fresh DMEM with 10% FBS. After 24 and 48 h, the virus-containing medium is collected and filtered with a 0.45 μm polyethersulfone filter, combined, and stored at −80 degrees C. until virus infection.

Cells are transduced with pLenti-CRISPR-V2 encoding the appropriate sgRNAs for the target genes using 2 μg/mL of polybrene followed by puromycin selection (1 μg/mL) for 4 days in the presence of ferrostatin-1 (1 μM). Protein knockout is verified via immunoblotting.

### Example 4: Use of C11-BODIPY to Show Lipid Peroxidation as an Indicator of Ferroptosis

Imaging assay: human cancer cells are seeded at 5,000 cells per well in a CellCarrier Ultra 96-well plate (Perkin-Elmer) in 150 μl of RPMI medium with 10% FBS. Cells are incubated for 24 h at 37° C. and then treated with the indicated compounds or DMSO (90 min, 37° C.). During the last 30 min of incubation, 60 nM DRAQ7 (Abcam), 1 μg ml⁻¹ Hoechst 33342 (ThermoFisher) and 1 μM BODIPY 581/591 C11 (ThermoFisher) dyes are added. Cells are imaged using an Opera Phenix High-Content Screening System (Perkin-Elmer) equipped with 405, 488, 560 and 647 nm lasers. Image analysis is conducted with Harmony High-Content Imaging and Analysis software (Perkin-Elmer).

### Use of C11-BODIPY to Show Lipid Peroxidation (Flow Cytometry Assay)

Human cancer cells are seeded at 15,000 cells per well in 96-well plates in RPMI medium with 10% FBS. After 48 h, culture media is replaced with 200 μl media containing either DMSO or the indicated inhibitor (10 μM) and 1 M anti-ferroptosis rescue agent (where indicated). Cultures are incubated at 37° C. for 2 h. Thirty minutes before the end of the incubation period, 10 μM BODIPY 581/591 C11 (Molecular Probes no. C10445) is added to cells. Cells are gathered in 200 μl PBS+0.1% BSA and subjected to flow cytometry analysis (BD FACSCanto II).

Example 5: Microdosing Tumors with Ferroptosis-Inducing Agents.

For the allograft study, cancer cell line-derived tumor cells were injected into the flanks of male C57BL6/J mice. Assays were initiated when the tumor diameter was approximately 6-7 mm.

Microdose drug delivery was performed for the assay described herein. The compounds in Table 3 were packed into device reservoirs using a tapered metal needle. Reservoirs were loaded for initial release of anti-ferroptosis rescue agent (M) (where included) followed by a 4-6 h delayed release of ferroptosis inducers. Devices were prepared for dose administration into mouse tumors. Devices delivered the ferroptosis inducing agent for 24-72 hours in the tissue. The tumor was then excised, and the tissue was snap frozen with liquid nitrogen. Tissue was sectioned using a standard cryotome, and tissue slices of 20 μm in thickness were collected from each reservoir for analysis by immunoassays, transcriptomics, and metabolomic assays.

TABLE 3
Microdosing Ferroptosis-Inducing Agents and Conditions

| | | | | Target |
|---|---|---|---|---|
| | Compound | | | concentration |
| Reservoir # | Name | Loading | Rescue agent | range (μM) |
| 1 | ferroptosis- | high | | 1-100 |
| | inducing | | | |
| | agent (C) | | | |
| 2 | ferroptosis- | high | M | 1-100 |
| | inducing | | | |
| | agent (C) | | | |
| 3 | ferroptosis- | low | | 1-10 |
| | inducing | | | |
| | agent (C) | | | |
| 4 | ferroptosis- | low | M | 1-10 |
| | inducing | | | |
| | agent (C) | | | |
| 5 | ferroptosis- | high | | 1-10 |
| | inducing | | | |
| | agent (B) | | | |
| 6 | ferroptosis- | high | M | 1-10 |
| | inducing | | | |
| | agent (B) | | | |
| 7 | ferroptosis- | high | | 1-10 |
| | inducing | | | |
| | agent (D) | | | |
| 8 | ferroptosis- | high | M | 1-10 |
| | inducing | | | |
| | agent (D) | | | |
| 9 | ferroptosis- | high | | 5-500 |
| | inducing | | | |
| | agent (E) | | | |
| 10 | ferroptosis- | high | M | 5-500 |
| | inducing | | | |
| | agent (E) | | | |
| 11 | ferroptosis- | high | | 100-150 |
| | inducing | | | |
| | agent (A) | | | |
| 12 | ferroptosis- | high | M | 100-150 |
| | inducing | | | |
| | agent (A) | | | |
| 13 | ferroptosis- | high | | 1-10 |
| | inducing | | | |
| | agent (F) | | | |
| 14 | ferroptosis- | high | M | 1-10 |
| | inducing | | | |
| | agent (F) | | | |
| 16 | Empty | | M | 1-10 |

### Example 6: System for In Vivo Ferroptosis-Inducing Agent Delivery

Ferroptosis-inducing agents and/or priming agents are administered systemically by injection to a mammal to establish local pharmacokinetics for the drugs. Representative drugs are tested include: ferroptosis-inducing agent (A), ferroptosis-inducing agent (C), ferroptosis-inducing agent (B), and anti-ferroptosis rescue agent (M). Representative animal models that can be used include for instance, those harboring tumors in a flammable membrane state.

A drug delivery system with microwells is loaded with approximately 1.5 micrograms of a ferroptosis-inducing agent (crystalline powder) per microwell. The system is loaded with the same drugs based on the results of the systemic testing. Each drug is loaded separately and in more than one concentration, as well as in combination. After 10, 12, 18, 24, 36 or 48 hours, devices are removed and histology of the tissue was examined to determine the effect of the ferroptosis-inducing agents on the tumor cells adjacent to each well. The effects of compounds eluted from microwells are assessed by different techniques. Tissue excised with the device is assayed by standard histopathological techniques, including immunohistochemistry and immunofluorescence. Ingrowth of tissue, ranging from 20 to about 300 microns, are visualized by staining tissue/device section by standard immuno-histochemistry (IHC) techniques, including hematoxylin & eosin (H&E) staining, or any nuclear cell stain such as DAPI. Mass spectrometry is used to measure local biomarkers indicative of an effect of a ferroptosis-inducing agent (e.g., mesenchymal cell state markers or PUFA concentration). Analysis for apoptosis, necrosis, mitotic cell death, and proliferation is conducted. The local microdose response is determined and used to define an appropriate therapeutic regime for the cancer.

Several methods for controlling the release/diffusion of ferroptosis-inducing agents into tissue, including precise spatial placement of microwells along device mantle; geometry and size of microwells; and formulation of released agents are developed. The device microwells from which the ferroptosis-inducing agents diffuse are engineered to expose only regions of tissue that are directly adjacent to the microwell opening, to the agent that is being released. This creates distinct local regions in the tissue in which the effects of compounds are assessed without interference of other compounds released from different microwells. Creation of discrete areas of drug are useful to assess the efficacy of the different agents, or combinations thereof, and/or dosages and/or times of release (sustained, pulsed, delayed, bolus followed by sustained, etc.).

Agents are released upward and diffused into a larger region, or released downward into a relatively smaller region of a target tissue. The precise control over the transport time as a function of distance from microwells provide a local concentration of a first agent as a function of distance from the microwell, at multiple time points following in vivo implantation.

Concentration gradient regions are defined as the distance from the microwell increases, the concentration of the agent being administered decreases. Cleaved caspase 3 positive cells as percent area of 3,3′-diaminobenzidine (DAB) staining as a function of distance from the microwell is one example of a functional readout from the implanted drug delivery system. The agent concentration gradient is formed approximately 100-250 μm from the microwell with tissue concentration as greatest in the regions closest to the microwell.

The system is used to deliver a microdose of a ferroptosis-inducing agent to a tissue in vivo. The system is also used to deliver a priming agent (e.g., anti-ferroptosis rescue agent (M)), followed by a ferroptosis-inducing agent (e.g., ferroptosis-inducing agent (C)) to a tissue in vivo to induce targeted cell death in the tissue. The system is also implanted directly into tumor of about 6 millimeters (mm) to about 7 mm in diameter to achieve a minimum amount of about 10 ng/mm² of drug at the site of the microwell for at least 4 hours.

### Example 7: Ferroptosis-Modulating Perturbation Affects Adipose Lipid Accumulation

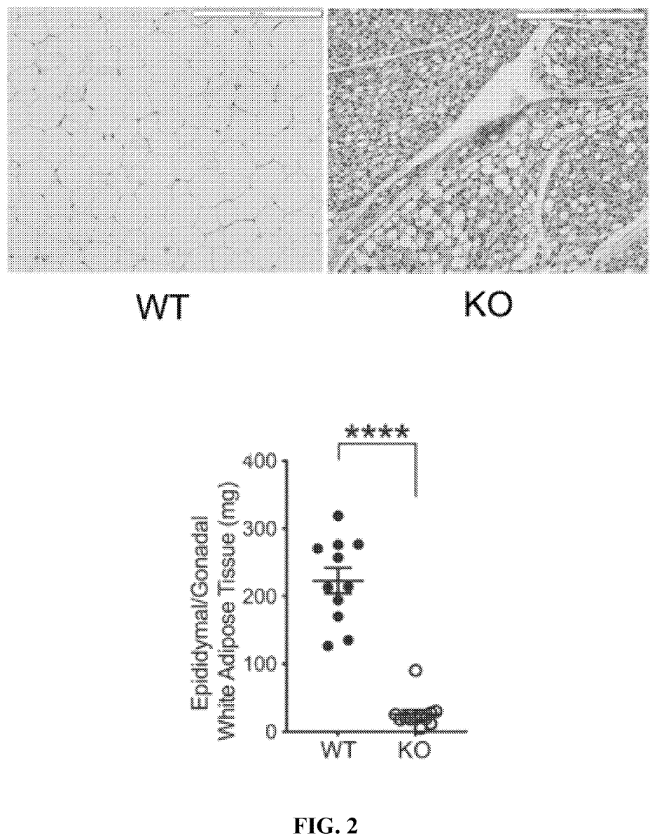

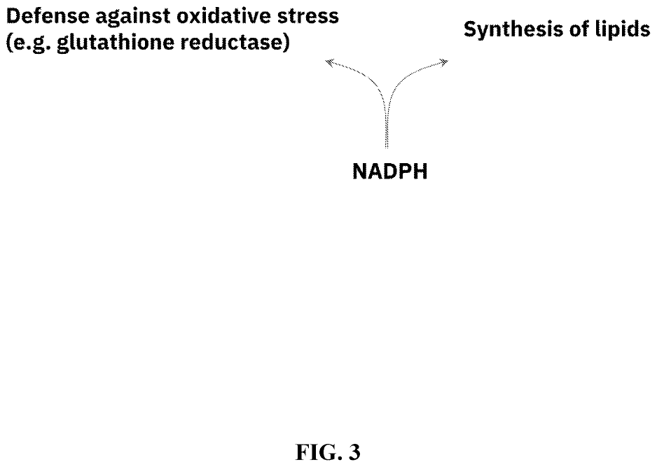

A specific ferroptosis-modulating knockout that results in ferroptosis was made in mice. There was significant loss of adiposity in the mutated mouse compared to the WT mouse as shown in FIG. 2. There is also a significant decrease in the amount of epididymal/gonadal white adipose tissue in WT mice compared to the knockout mouse. This decrease in adipose tissue can be also seen by pharmacological modulation. The perturbations are safe and tolerated. Partial loss-of-function result in a lean phenotype when mice are subjected to high fat diet (HFD). Constitutive activation of the pathway is known to cause fatty liver and hepatomegaly in mice.

### Example 8: a Ferroptosis Agent Treatment Reproduces Weight-Loss Phenotype

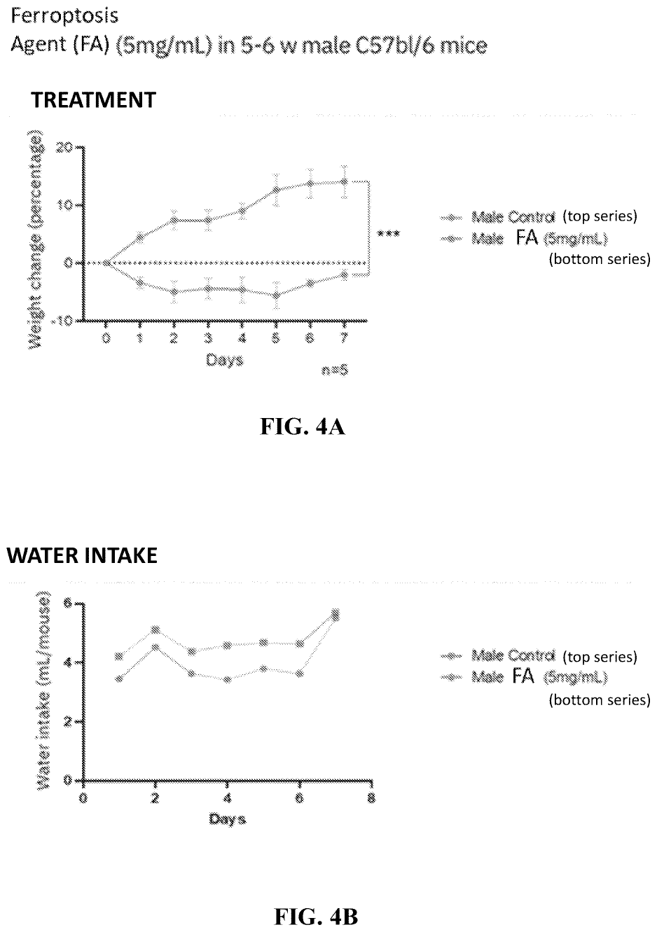

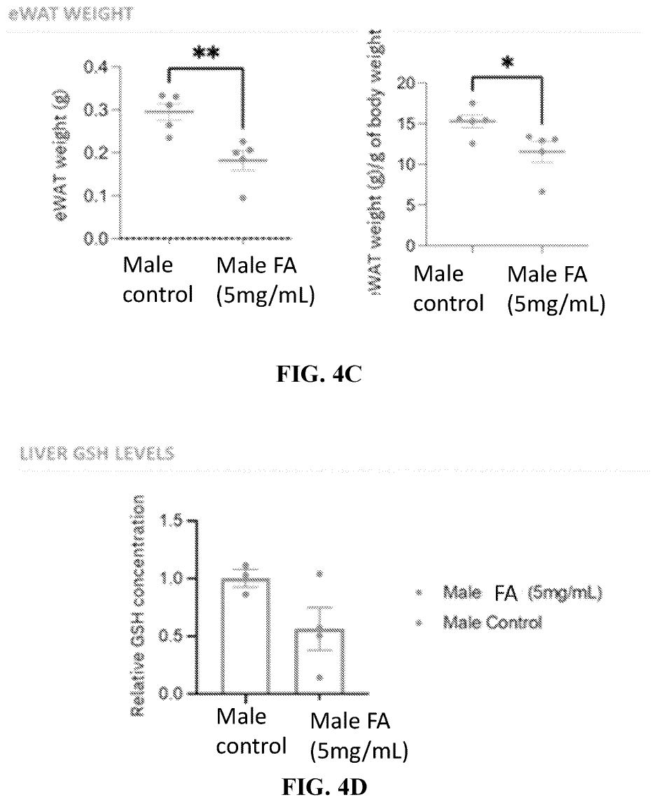

Male C57Bl/6 mice (5-6 weeks old) were treated with (5 mg/mL) of a ferroptosis agent to show weight loss with a glutamate-cysteine ligase inhibitor. Glutathione (GSH) is synthesized by the sequential actions of rate-limiting glutamate-cysteine ligase (GCL) and glutathione synthase. FIGS. 4A-4C show that mice treated with the ferroptosis agent had decreased weight change (percentage) and decreased epididymal white adipose tissue (eWAT) weight (g) and eWAT weight (g/g) of body weight. Additionally, FIG. 4B shows the water intake between control and the ferroptosis agent treatment groups as well as a decrease in liver GSH levels in ferroptosis agent treated mice as compared to the control group, which was water only (FIG. 4D). This data shows inhibiting glutamate-cysteine ligase can lead to weight loss.

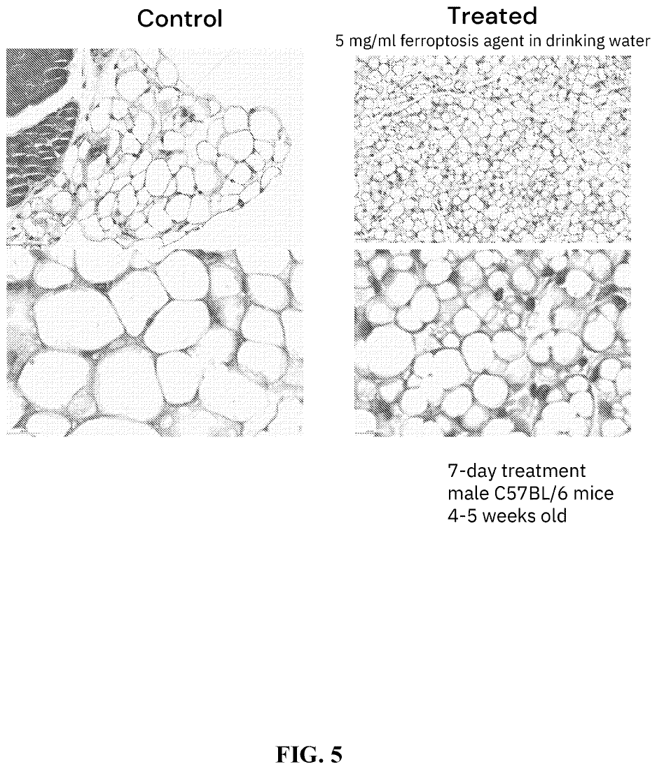

Similarly, FIG. 5 shows histological samples of male C57Bl/6 mice (4-5 weeks old) treated for 7 days with 5 mg/mL of a ferroptosis agent and of control mice treated with water. In the treated sample, there is decreased adiposity observed in the histological samples.

### Example 9: Ferroptosis Agent Treatment in Mice on High Fat Diet (HED)

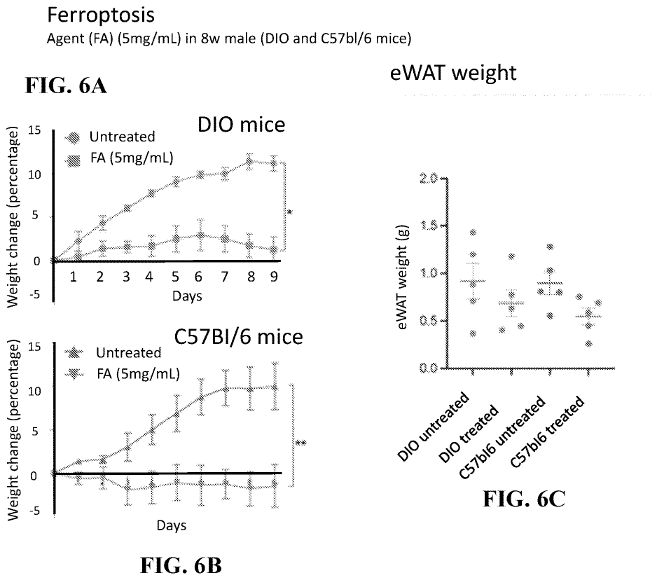

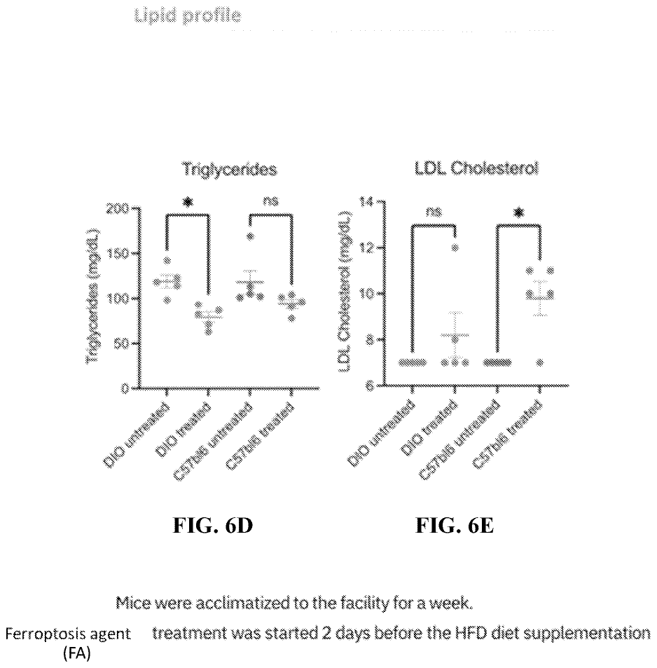

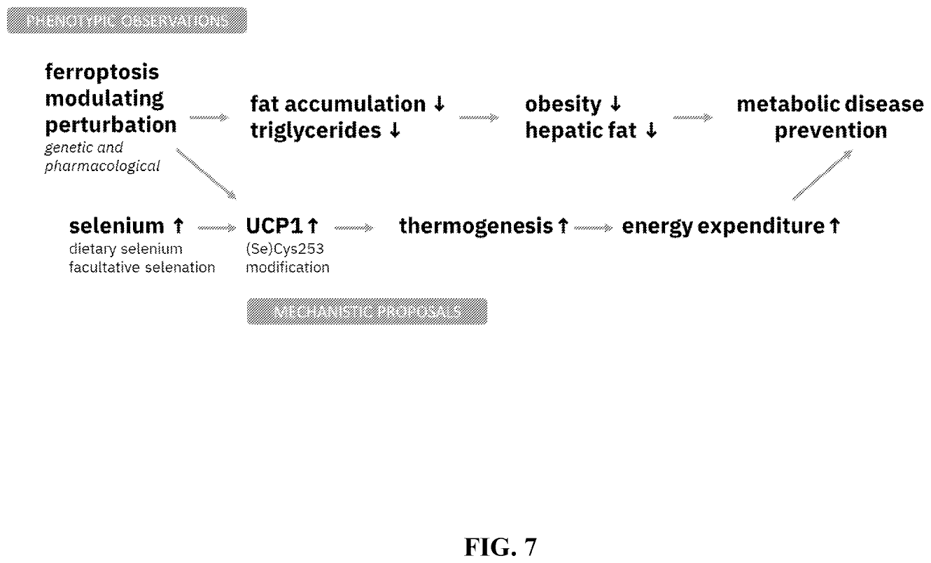

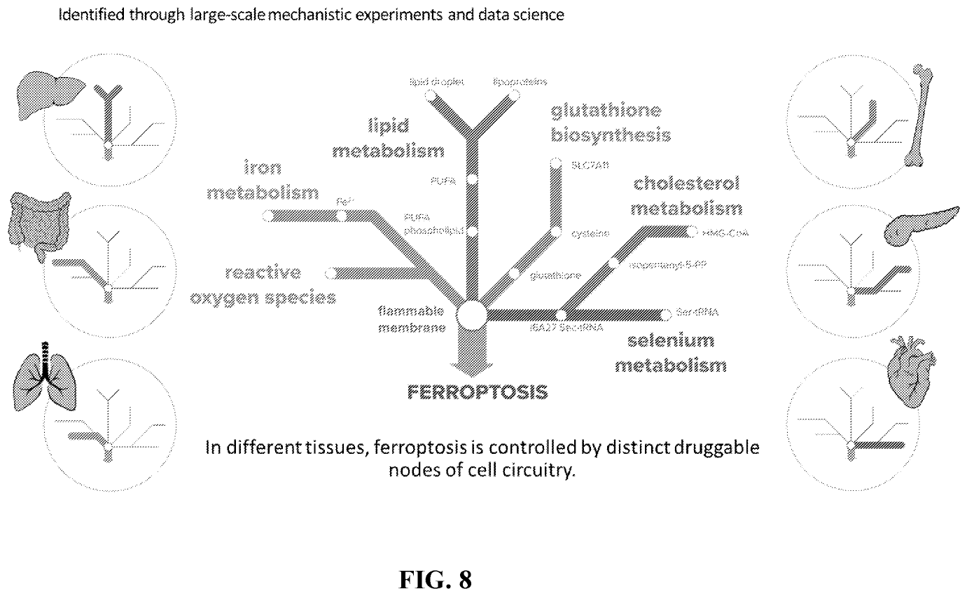

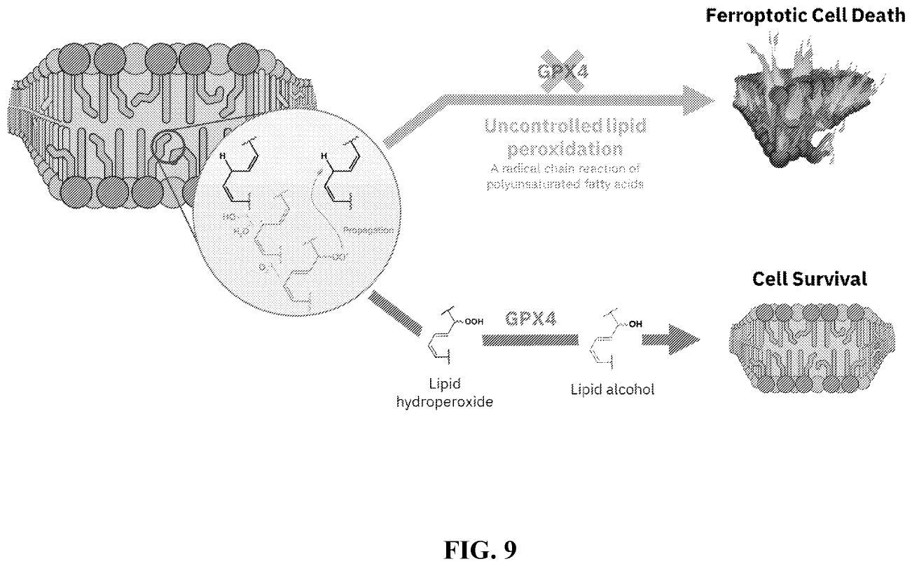

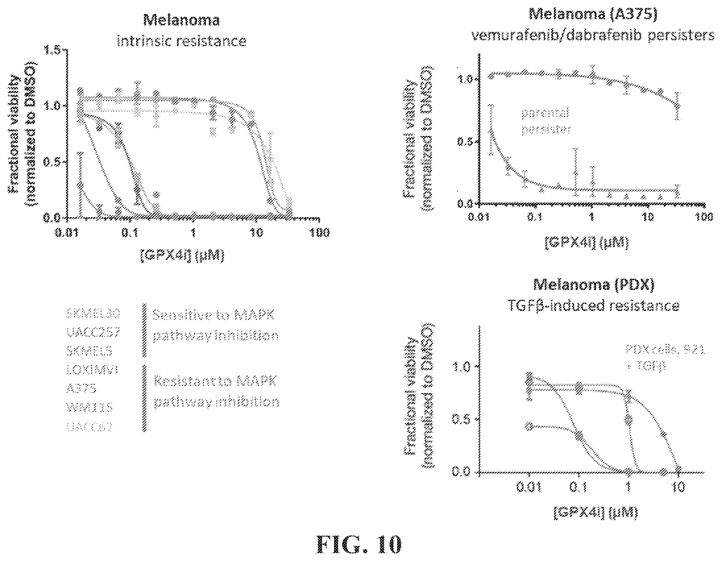

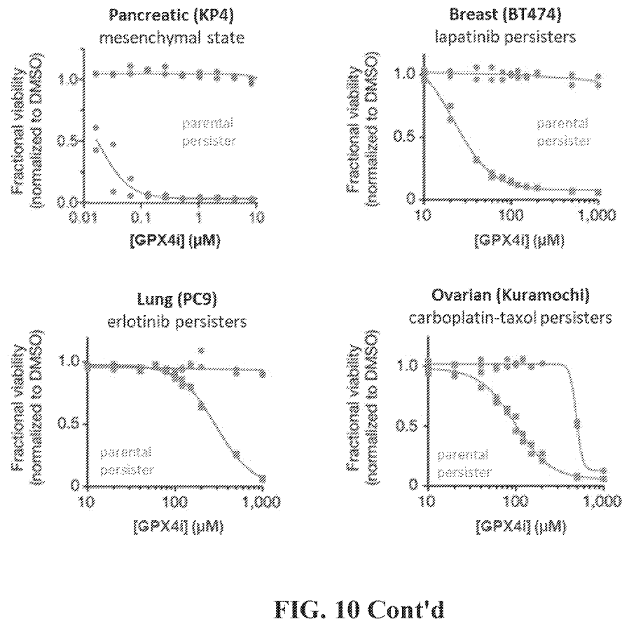

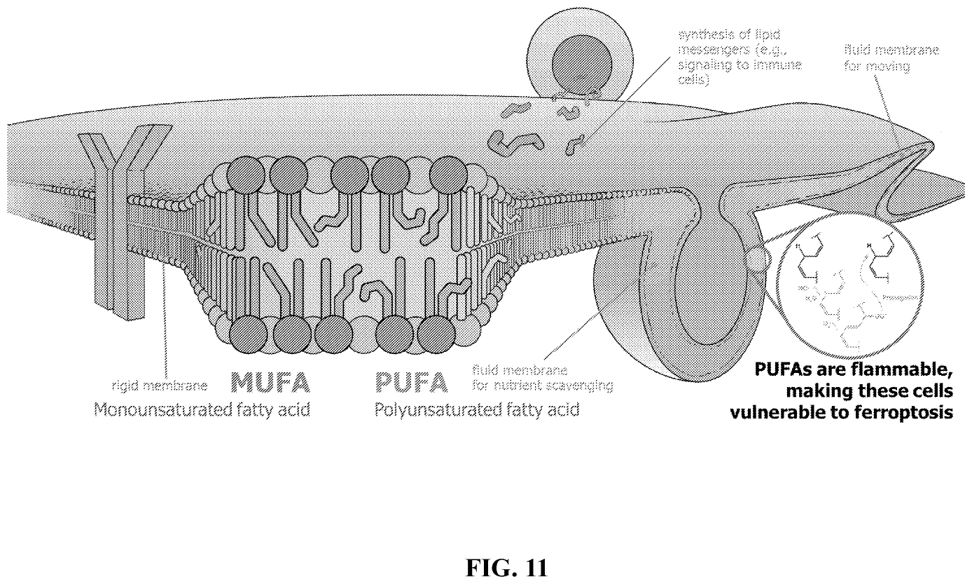

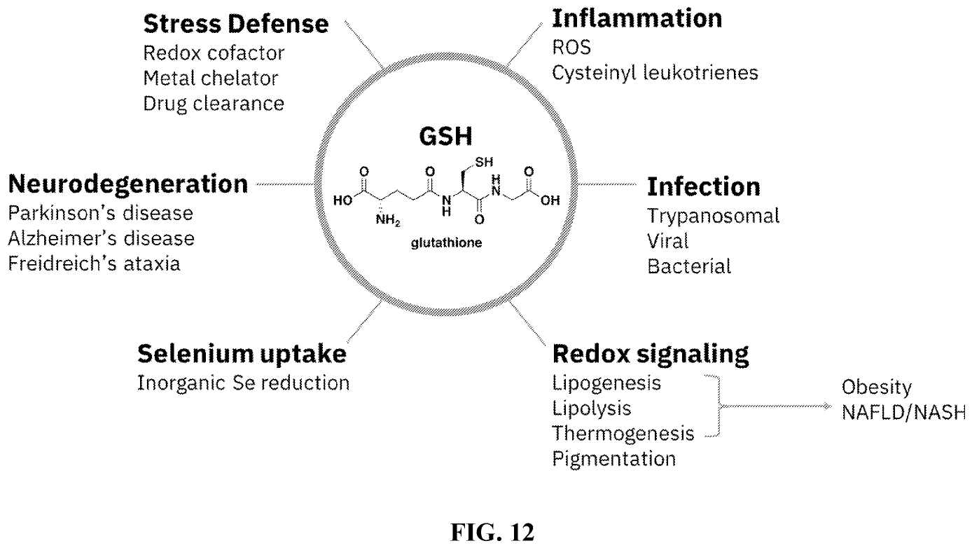

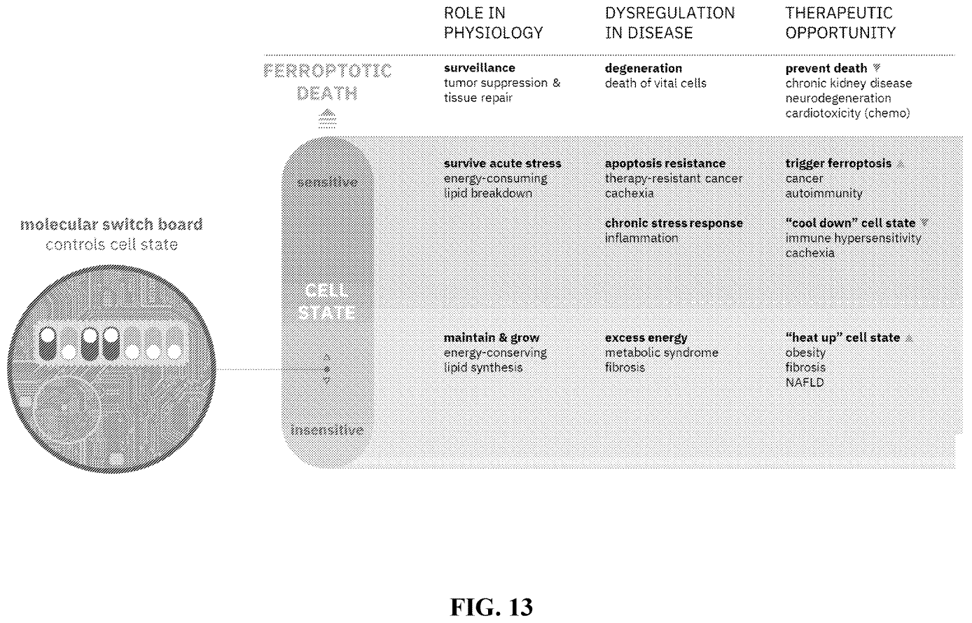

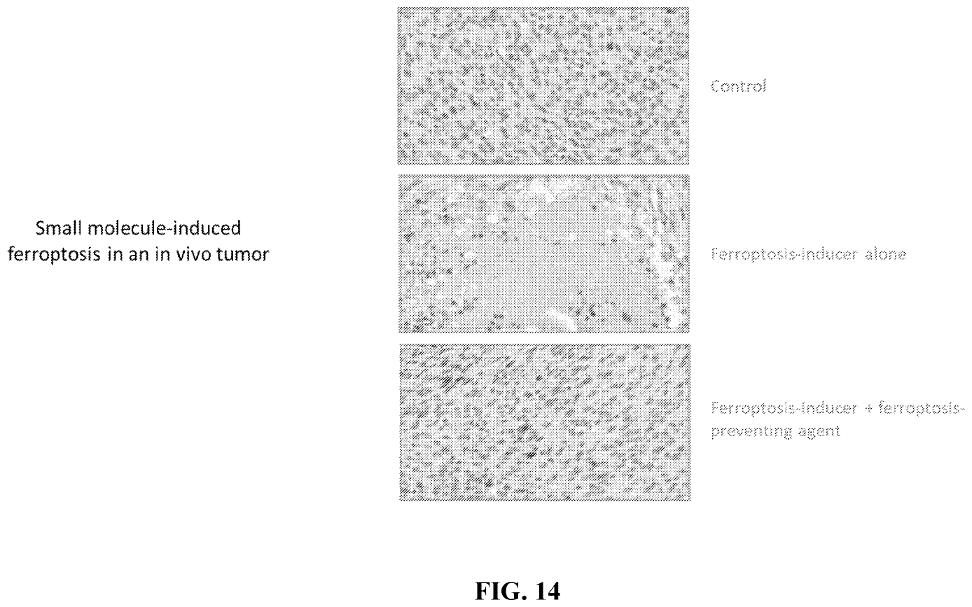

Male C57Bl/6 mice and diet induced obese (DIO) mice were treated with 5 mg/ml of a ferroptosis agent to show weight loss with a high fat diet. The mice were 8 week old male mice. The mice were acclimatized to the facility for a week. The ferroptosis agent treatment was started 2 days before the HFD diet supplementation. FIGS. 6A-6C show that mice (both the C57Bl/6 and DIO mice) treated with the ferroptosis agent had decreased weight change (percentage) and decreased epididymal white adipose tissue (eWAT) weight (g). Additionally, FIG. 6D-6E shows the triglyceride levels were decreased in the DIO treated mice and the LDL cholesterol levels were increased in C57Bl/6 mice treated with the ferroptosis agent. This data shows that mice did not gain significant weight on a HFD diet when treated with the ferroptosis agent.

What is claimed is:

1. A method of inducing weight loss, fat loss, or both, in a subject, the method comprising administering to the subject a ferroptosis inducing agent in an amount sufficient to induce the weight loss, the fat loss, or both.

2. The method of claim 1, that is a method of inducing weight loss.

3. The method of claim 1, that is a method of inducing fat loss.

4. The method of claim 1, that is a method of inducing weight loss and fat loss.

5. The method of claim 1, further comprising, before the administering to the subject the ferroptosis inducing agent, administering a priming agent to the subject.

6. The method of claim 1, wherein an adipose tissue in the subject, after the administrating of the ferroptosis inducing agent to the subject, diminishes by weight, by volume, or both, relative to a weight, volume, or both of the adipose tissue in the subject before the administering.

7. The method of claim 6, wherein the adipose tissue in the subject, after the administering to the subject of the ferroptosis inducing agent, diminishes by volume relative to a volume of the adipose tissue before the administering, as determined by a pinch clamp test, by a dual x-ray absorptiometry (DEXA) scan, a diminished waist measurement, a computerized axial tomography (CAT) test, or any combination thereof.

8. The method of claim 6, wherein the adipose tissue in the subject, after the administering to the subject of the ferroptosis inducing agent, diminishes by weight relative to a weight of the adipose tissue before the administering.

9. The method of claim 8, wherein the diminishes by weight is determined by the subject weighing less in total body weight after the administering of the ferroptosis inducing agent as compared to the total body weight of the subject before the administering of the ferroptosis inducing agent.

10. The method of claim 8, wherein the diminishes by weight is determined by comparing DEXA scan results of the subject taken before and after the administering to the subject of a ferroptosis inducing agent.

11. The method of claim 1, further comprising that a cell size, a cell volume, or both, of a plurality of cells in an adipose tissue of the subject is diminished after the administering to the subject of a ferroptosis inducing agent, relative to a cell size, a cell volume, or both, of a plurality of cells in the adipose tissue of the subject before the administering.

12. The method of claim 1, wherein the subject is a human.

13. The method of claim 1, wherein the ferroptosis inducing agent is administered orally.

14. The method of claim 1, wherein the administration occurs for about: 1 day, 2 days, 3 days, 4 days, 5 days, one week, 10 days, two weeks, 15 days, 20, days, three weeks, 25 days, 30 days, one month, two months, three months, four months, five months, six months, one year, two years, three years, four years, five years, for life, or as needed.

15. The method of claim 14, wherein the ferroptosis inducing agent is administered in an amount ranging from about 0.1 ng to about 25,000 mg.

16. The method of claim 14, wherein the ferroptosis inducing agent is administered in an amount of about: 1 ng, 10 ng, 100 ng, 1 microgram, 10 micrograms, 100 micrograms, 1 mg, 10 mg, 100 mg, 1 g, 2 g, 3 g, 4 g, 5 g, 6 g, 7 g, 8 g, 9 g, 10 g, 15 g, 20 g, or 25 g.

17. The method of claim 14, wherein the ferroptosis inducing agent is administered in an amount ranging from about 1 ng/kg to 1,000 mg/kg, wherein mg is mg of the ferroptosis inducing agent, and kg is kg of body weight of the subject.

18. The method of claim 1, wherein the ferroptosis inducing agent is administered orally.

19. The method of claim 1, wherein the ferroptosis inducing agent is administered by one of the following routes: oral administration, subcutaneous, intradermal, intramuscular, inhalation, intravenous, intraperitoneal, intracranial, intrathecal, transcutaneous, rectal or directly to a fatty tissue.

20. The method of claim 1, that treats at least one of: obesity, metabolic syndrome, elevated blood glucose, a diabetes, diabetes type 2, diabetes type 3, insulin resistance, high blood pressure, a cardiovascular disease, a coronary artery disease, a cerebrovascular disease, a stroke, a rheumatic heart disease, an arteriosclerosis, an atherosclerosis, a liver disease, a fatty liver disease, a nonalcoholic fatty liver disease (NAFLD), a nonalcoholic steatohepatitis (NASH), or any combination thereof.

21. The method of claim 1, wherein the subject is a human male.

22. The method of claim 1, wherein the subject is a human female.

23. The method of claim 1, wherein the subject ranges from about 1 year of age to about 18 years of age.

24. The method of claim 1, wherein the subject ranges from about 18 years of age to about 120 years of age.

25. The method of claim 1, wherein the subject is a subject in need thereof.

26. The method of claim 1, wherein the ferroptosis inducing agent is comprised in a pharmaceutical composition that comprises a pharmaceutically acceptable: excipient, carrier, or diluent.

27. The method of claim 26, wherein the pharmaceutical composition is in unit dose form.

28. The method of claim 1, further comprising administering a further therapeutic to the subject.

29. The method of claim 28, wherein the further therapeutic is administered concurrently with the ferroptosis inducing agent.

30. The method of claim 28, wherein the further therapeutic is administered consecutively with the ferroptosis inducing agent.

31. The method of claim 1, wherein the ferroptosis inducing agent is administered directly to a fatty tissue.

32. The method of claim 31, wherein the administering is continuously.

33. The method of claim 32, wherein the administering is for about: 1/2 hour, 1 hour, 2 hours, 3 hours, four hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 2 days, 3 days, 4 days, 5 days, 6 days, or one week.

34. The method of claim 31, wherein the fatty tissue is heated to a temperature of about; 23, 24, 25, 26, 27, 28, 29, 30, 31, 32, or 33 degrees Celsius.

35. The method of claim 31, wherein the ferroptosis inducing agent is administered in the form of an aqueous solution.

36. The method of claim 35, wherein a concentration of the ferroptosis inducing agent in the aqueous solution is from about 0.1 nM to about 500 microM.

37. The method of claim 1, wherein the administration results in reducing an adipose cell size, or number of adipose cells of the subject, wherein the reduction is determined by measuring the adipose cell size, or number of adipose cells, before and after the administration of the therapeutic amount of the ferroptosis-modulating agent, optionally employing a microscopic evaluation of a biopsy.

38. The method of claim 1, wherein the method further comprises reducing an epididymal white adipose tissue of the subject.

39. The method of claim 1, wherein the subject is overweight or obese.

40. The method of claim 1, wherein the subject is obese and has a BMI of about 30.0 or higher.

41. The method of claim 1, wherein the subject is overweight and has a BMI of about 25.0 to about 30.

42. The method of claim 28, wherein the further therapeutic has glucagon-like peptide-1 (GLP-1) receptor agonist activity.

43. The method of claim 42, wherein the further therapeutic comprises semaglutide, dulaglutide, liraglutide, exenatide, tirzepatide, or any combination thereof.

44. A method of inducing, regulating, or modulating weight loss, body composition, or fat loss or fat reduction in a subject, the method comprising administering to the subject a therapeutically effective amount of a ferroptosis-modulating agent, a glutamate-cysteine ligase (GCL) modulating agent, an agent that binds to GCL, an agent that inhibits GCL, or any combination thereof to the subject, thereby inducing, regulating, or modulating the weight loss, body composition, or fat loss or fat reduction in the subject.

45. The method of claim 44, wherein the subject is a human.

46. The method of claim 45, wherein the subject is a male.

47. The method of claim 44, wherein the ferroptosis-modulating agent, the glutamate-cysteine ligase (GCL) modulating agent, the agent that binds to GCL, the agent that inhibits GCL, or any combination thereof is delivered orally as a solution having a concentration of at least about: 5 mg agent per mL of the solution.

48. The method of claim 44, wherein the administration is discontinuous and the administration occurs daily for about: 5 days, 10 days, 15 days, 20, days, 25 days, 30 days, two months, three months, four months, five months, six months, or one year.

49. The method of claim 1, wherein the ferroptosis inducing agent is selected from the group consisting of: (1S,3R)-RSL3, ML-162, ML-210, JKE-1674, JKE-1716, erastin, jacaric acid, trigonelline, glutamate, sulfasalazine, auranofin, brusatol, sorafenib, sorafenib-d3, sorafenib tosylate, trigonelline, FIN56, FINO2, CIL56, dihydroisotanshinone I, GPX4-IN-3, a sulfoximine, BSO, and a salt of any of these.

50. The method of claim 1, wherein the administration results in reducing an adipose cell size of the subject, wherein the reduction is determined by measuring the adipose cell size before and after the administration of the ferroptosis-inducing agent, optionally employing a microscopic evaluation of a biopsy.

51. The method of claim 1, wherein the administration results in reducing the number of adipose cells of the subject, wherein the reduction is determined by measuring the number of adipose cells before and after the administration of the therapeutic amount of the ferroptosis-modulating agent, optionally employing a microscopic evaluation of a biopsy.

52. The method of claim 50 or claim 51, wherein the method comprises reducing an epididymal white adipose tissue of the subject.

53. The method of claim 52, wherein the subject is overweight or obese.

54. The method of claim 53, wherein the subject is obese and has a BMI of about 30.0 or higher.

55. The method of claim 53, wherein the subject is overweight and has a BMI of about 25.0 to about 30.

56. The method of any preceding claim, wherein the subject is administered concurrently or consecutively an additional agent or therapy.

57. The method of claim 56, wherein the additional agent or therapy is selected from the group consisting of: a GLP-1 agonist, a gastric inhibitory polypeptide analog, naltrexone-bupropion or a salt thereof, orlistat or a salt thereof, phentermine or a salt thereof, phentermine-topiramate or a salt thereof, selenium, an exercise therapy, a surgery, a diet modification, and any combination thereof.

58. The method of claim 57, wherein the additional agent or therapy comprises administering the GLP-1 agonist that comprises dulaglutide, exenatide, semaglutide, liraglutide, lixisenatide, or a salt of any of these.

59. The method of claim 57, wherein the additional agent or therapy comprises administering the gastric inhibitory polypeptide that comprises tirzepatide or a derivative thereof.

60. The method of claim 57, wherein additional agent or therapy comprises the surgery that comprises a gastric sleeve, a gastric bypass, an adjustable gastric band, a ball that is placed in the stomach, or any combination thereof.

61. The method of any preceding claim, wherein the subject has not been diagnosed with a cancer.

62. The method of any preceding claim, wherein the subject is a subject in need thereof.

63. The method of any preceding claim, wherein the subject has diabetes type I, diabetes type II, diabetes type IIIa, or a metabolic syndrome.

64. The method of claim 44, wherein the ferroptosis-modulating agent, the glutamate-cysteine ligase (GCL) modulating agent, the agent that binds to GCL, the agent that inhibits GCL, or any combination thereof is in the form of a pharmaceutical composition that further comprises a pharmaceutically acceptable: carrier, excipient, or diluent.

65. The method of claim 64, wherein the pharmaceutical composition is in unit dose form.

66. A composition comprising i) a ferroptosis-modulating agent, a glutamate-cysteine ligase (GCL) modulating agent, an agent that binds to GCL, an agent that inhibits GCL; and ii) a GLP-1 agonist or a salt thereof, a gastric inhibitory polypeptide analog, a naltrexone-bupropion or a salt thereof, an orlistat or a salt thereof, a phentermine or a salt thereof, and a phentermine-topiramate or a salt thereof, or a selenium or a salt thereof.

67. A composition comprising i) a glutamate-cysteine ligase (GCL) modulating agent, and ii) a GLP-1 agonist or a salt thereof, a gastric inhibitory polypeptide analog, a naltrexone-bupropion or a salt thereof, an orlistat or a salt thereof, a phentermine or a salt thereof, and a phentermine-topiramate or a salt thereof, or a selenium or a salt thereof.

68. The composition of claim 66 or claim 67, which is a pharmaceutical composition.

69. The pharmaceutical composition of claim 68, that is in unit dose form.

70. The composition of any one of claims 66-69, for use in the treatment of a disease or condition.

71. The composition of any one of claims 66-69, for use in the treatment of a kidney disease or condition.

72. The composition of any one of claims 66-69, for use in the treatment of a symptom associated with a kidney disease or condition.

73. The composition for use of claim 70, wherein the disease or condition is weight loss, weight maintenance, an iron metabolism associated disease or condition, or a disease or condition associated with high, low, or abnormal cholesterol.

74. A method of modulating iron metabolism in a plurality of cells in a subject, the method comprising administering to the subject a therapeutically effective amount or an effective amount of a ferroptosis-inducing agent, a glutamate-cysteine ligase (GCL) modulating agent, an agent that binds to GCL, an agent that inhibits GCL, or any combination thereof, thereby modulating the iron metabolism in the subject and thereby inducing ferroptosis in the subject.

75. The method of claim 74, wherein immediately after the modulating, a majority of cells of the plurality of cells remain alive.

76. A method of modulating iron metabolism in a plurality of cells in a subject, the method comprising administering to the subject a therapeutically effective amount or an effective amount of a ferroptosis-inducing agent, thereby modulating the iron metabolism in the subject.

77. The method of claim 76, wherein immediately after the modulating, a majority of cells of the plurality of cells remain alive.

78. A method of treating, inducing, regulating, or modulating iron metabolism or a disease or condition associated with iron metabolism in a subject, the method comprising administering to the subject a therapeutically effective amount or an effective amount of a ferroptosis-modulating agent, a glutamate-cysteine ligase (GCL) modulating agent, an agent that binds to GCL, an agent that inhibits GCL, or any combination thereof, thereby treating, inducing, regulating, or modulating the iron metabolism or the disease or condition associated with the iron metabolism in the subject.

79. The method of claim 78, wherein the subject is a human.

80. The method of claim 78, wherein subject is a subject in need thereof.

81. The method of claim 78, wherein a therapeutically effective amount of the ferroptosis-modulating agent is added.

82. A method of modulating cholesterol metabolism in a subject, the method comprising administering to the subject a therapeutically effective amount or an effective amount of a ferroptosis-modulating agent, a glutamate-cysteine ligase (GCL) modulating agent, an agent that binds to GCL, an agent that inhibits GCL, or any combination thereof, thereby modulating cholesterol metabolism in the subject.

83. The method of claim 82, wherein the subject is a human.

84. The method of claim 82, wherein the subject is a subject in need thereof.

85. The method of claim 84, wherein the ferroptosis inducing agent is administered.

86. A method of modulating cholesterol metabolism in a subject, the method comprising administering to the subject a therapeutically effective amount or an effective amount of a ferroptosis-inducing agent, thereby modulating the cholesterol metabolism in the subject.

87. The method of claim 86, wherein the subject is a human.

88. The method of claim 86, wherein the subject is a subject in need thereof.

89. The method of claim 86, wherein the ferroptosis-inducing agent is administered.

90. A method of treating, inducing, regulating, modulating, or diminishing cholesterol metabolism or a disease associated with cholesterol metabolism in a subject, the method comprising administering to the subject a therapeutically effective amount or an effective amount of a ferroptosis-modulating agent, a glutamate-cysteine ligase (GCL) modulating agent, an agent that binds to GCL, an agent that inhibits GCL, or any combination thereof, thereby treating, inducing, regulating, modulating, or diminishing the cholesterol metabolism or the disease or condition associated with the cholesterol metabolism in the subject.

91. The method of claim 90, wherein the subject is a human.

92. The method of claim 90, wherein the subject is a subject in need thereof.

93. The method of claim 90, wherein the ferroptosis-modulating agent is administered.

94. A method of treating, inducing, regulating, modulating, diminishing, or increasing a reactive oxygen species or a disease or condition associated with a reactive oxygen species in a subject, the method comprising administering to the subject a therapeutically effective amount or an effective amount of a ferroptosis-modulating agent, a glutamate-cysteine ligase (GCL) modulating agent, an agent that binds to GCL, an agent that inhibits GCL, or any combination thereof, thereby treating, inducing, regulating, modulating, diminishing, or increasing the reactive oxygen species or the disease or condition associated with the reactive oxygen species in the subject.

95. The method of claim 94, wherein the subject is a human.

96. The method of claim 94, wherein the subject is a subject in need thereof.

97. The method of claim 94, wherein the ferroptosis-modulating agent is administered.

98. A method of increasing a reactive oxygen species in a subject, the method comprising administering to the subject a therapeutically effective amount or an effective amount of a ferroptosis-modulating agent, a glutamate-cysteine ligase (GCL) modulating agent, an agent that binds to GCL, an agent that inhibits GCL, or any combination thereof, thereby increasing the reactive oxygen species in the subject.

99. The method of claim 98, wherein the subject is a human.

100. The method of claim 98, wherein the subject is a subject in need thereof.

101. The method of claim 98, wherein the ferroptosis-modulating agent is administered.

102. A method of promoting at least one of: treating, surviving, modulating, or diminishing an acute stress; a chronic stress response; an immune hypersensitivity; cachexia; kidney disease; neurodegeneration; cardiotoxicity; or cardiotoxicity caused at least in part by chemotherapy; in a subject, the method comprising administering to the subject a therapeutically effective amount or an effective amount of a ferroptosis-modulating agent, a glutamate-cysteine ligase (GCL) modulating agent, an agent that binds to GCL, an agent that inhibits GCL, or any combination thereof, thereby treating, inducing, regulating, modulating, diminishing, or increasing the acute stress; a chronic stress response; an immune hypersensitivity; cachexia; kidney disease; neurodegeneration; cardiotoxicity; or cardiotoxicity cause at least in part by chemotherapy in the subject.

103. The method of claim 102, wherein the subject is a human.

104. The method of claim 102, wherein the subject is a subject in need thereof.

105. The method of claim 102, wherein the ferroptosis-modulating agent is administered.

106. A method of promoting at least one of: treating a kidney disease in a subject, the method comprising administering to the subject a therapeutically effective amount or an effective amount of a ferroptosis-modulating agent, a glutamate-cysteine ligase (GCL) modulating agent, an agent that binds to GCL, an agent that inhibits GCL, or any combination thereof, thereby treating the kidney disease in the subject.

107. The method of claim 106, wherein the subject is a human.

108. The method of claim 106, wherein the subject is a subject in need thereof.

109. The method of claim 106, wherein the ferroptosis-modulating agent is administered.

110. A method of promoting at least one of: treating a kidney disease in a subject, the method comprising administering to the subject a therapeutically effective amount or an effective amount of a ferroptosis-inducing agent, thereby treating the kidney disease in the subject.

111. The method of claim 110, wherein the subject is a human.

112. The method of claim 110, wherein the subject is a subject in need thereof.

113. The method of claim 110, wherein the ferroptosis-inducing agent is administered.

114. The method of any one of claims 74-110, wherein the method comprises reducing an epididymal white adipose tissue of the subject.

115. The method of claim 114, wherein the subject is overweight or obese.

116. The method of claim 115, wherein the subject is obese and has a BMI of 30.0 or higher.

117. The method of claim 115, wherein the subject is overweight and has a BMI of 25.0 to <30.

118. The method of any one of claims 74-117, wherein the subject is administered concurrently or consecutively an additional agent or therapy.

119. The method of claim 118, wherein the additional agent or therapy is selected from the group consisting of: a GLP-1 receptor agonist, a gastric inhibitory polypeptide analog, naltrexone-bupropion or a salt thereof, orlistat or a salt thereof, phentermine or a salt thereof, phentermine-topiramate or a salt thereof, selenium, an exercise therapy, a surgery, a diet modification, and any combination thereof.

120. The method of claim 119, wherein the additional agent or therapy comprises administering the GLP-1 receptor agonist that comprises dulaglutide, exenatide, semaglutide, liraglutide, lixisenatide, or a salt of any of these.

121. The method of claim 119, wherein the additional agent or therapy comprises administering the gastric inhibitory polypeptide that comprises tirzepatide or a derivative thereof.

122. The method of claim 119, wherein additional agent or therapy comprises the surgery that comprises a gastric sleeve, a gastric bypass, an adjustable gastric band, a ball that is placed in the stomach, or any combination thereof.

123. The method of any one of claims 74-122, wherein the subject has not been diagnosed with a cancer.

124. The method of any one of claims 74-122, wherein the subject is a subject in need thereof.

125. The method of any one of claims 74-122, wherein the subject has diabetes type I, diabetes type II, diabetes type IIIa, or a metabolic syndrome.

126. The method of any one of claims 74-122, wherein the agent is in the form of a pharmaceutical composition that further comprises a pharmaceutically acceptable: carrier, excipient, or diluent.

127. The method of claim 126, wherein the pharmaceutical composition is in unit dose form.

128. The method of any preceding claim, wherein the agent is a compound or a salt thereof in Table 1.

129. The method of any preceding claim, wherein the agent is in the form of a pharmaceutical composition that further comprises a pharmaceutically acceptable: carrier, excipient, or diluent.

130. The method of claim 46, wherein the pharmaceutical composition is in unit dose form.

131. A composition comprising i) a ferroptosis-modulating agent, a glutamate-cysteine ligase (GCL) modulating agent, an agent that binds to GCL, an agent that inhibits GCL; and ii) a GLP-1 agonist or a salt thereof, a gastric inhibitory polypeptide analog, a naltrexone-bupropion or a salt thereof, an orlistat or a salt thereof, a phentermine or a salt thereof, and a phentermine-topiramate or a salt thereof, or a selenium or a salt thereof.

132. The composition of claim 131, which is a pharmaceutical composition.

133. The pharmaceutical composition of claim 132, that is in unit dose form.

134. The composition of any one of claims 131-133, for use in the treatment of a disease or condition.

135. The composition of any one of claims 131-133, for use in the treatment of a kidney disease or condition.

136. The composition of any one of claims 131-133, for use in the treatment of a symptom associated with a kidney disease or condition.

137. The composition for use of claim 134, wherein the disease or condition is weight loss, weight maintenance, an iron metabolism associated disease or condition, or a disease or condition associated with high, low, or abnormal cholesterol.
